(19) United States
(12) Patent Application Publication
(10) Pub. No.: US 2005/0148495 A1
(43) Pub. Date: Jul. 7, 2005

(54) Methods and materials including for treating acne

(76) Inventors: Lewis H. Lambert JR., Fremont, CA (US); Geertrui F. A. Vanhove, Berkeley, CA (US)

Correspondence Address: Janet M. McNicholas, Ph.D.;McAndrews, Held & Malloy, Ltd., 34th Floor, 500 West Madison Street, Chicago, IL, 60661, US

(21) Appl. No.: 10/898,636

(22) Filed: Jul. 23, 2004

Related U.S. Application Data
(60) Provisional application No. 60/489,618, filed on Jul. 23, 2003.
(60) Provisional application No. 60/554,705, filed on Mar. 19, 2004.

(51) Int. Cl. A61K 38/17; A61K 31/65; A61K 31/203; A61K 31/192

(52) U.S. Cl. 514/2; 514/154; 514/559; 514/568; 424/401

(57) ABSTRACT

The present invention provides novel methods and materials including for treating acne and including methods comprising administering to a subject a composition comprising a therapeutically effective amount of XMP.629 or a pharmaceutically acceptable salt or derivative thereof.

### RELATED APPLICATIONS

This application claims the benefit of U.S. Provisional Application No. 60/489,618 filed Jul. 23, 2003, and U.S. Provisional Application No. 60/554,705 filed Mar. 14, 2004, the disclosures of which are incorporated by reference herein in its entirety.

### BACKGROUND OF THE INVENTION

Acne (acne vulgaris) is a common skin disorder, and is especially prevalent in the United States. According to estimates provided by the U.S. Census Bureau for 2000, the total number of Americans affected with acne is approximately 41 to 48 million. The onset of acne usually occurs at or just beyond puberty and can persist for 6-14 years and sometimes longer. The American Academy of Dermatology has reported that 85% to 100% of those aged 12-24 are affected by either intermittent or persistent acne, which in a number of adolescents results in scarring attributed to acne (Bershad, The Mount Sinai Journal of Medicine, Vol. 68, p. 279-286, 2001; White, Journal of American Academy of Dermatology, Vol. 39, p. S34-37, 1998). Furthermore, acne can remain problematic into the third to fifth decades of life, particularly in women. Tan et al., (Journal of American Academy of Dermatology, Vol. 44 (Supplement 3), p. 439-445, 2001), has reported that approximately 3% of all male adults and 12% of all female adults in the U.S. suffer from acne.

The basic acne lesion, called the comedo or comedone, is an enlarged hair follicle plugged with oil and bacteria. Common symptoms or signs of acne include open comedones (blackheads) and closed comedones (whiteheads), which are symptoms or signs of mild acne whereas papules are inflamed lesions that usually appear as small, pink bumps on the skin and can be tender to the touch. Pustules or pimples are inflamed, pus-filled lesions that can be red at the base. Nodules are large, painful, solid lesions that are lodged deep within the skin and cysts are deep, inflamed, pus-filled lesions that can cause pain and scarring. The clinical symptoms or signs of the pathophysiologic events in acne range from non-inflammatory open and closed comedones to inflammatory papules, pustules, and nodules. Most patients present with a mixture of non-inflammatory and inflammatory lesions, whereas some patients present with predominantly one type of lesion over the other.

The pathogenesis of acne vulgaris centers around pilosebaceous units; these units are largest and most numerous on the face, upper back, chest and upper outer arms. Pilosebaceous units comprise of a sebaceous (oil) gland that is connected to a hair follicle. Sebaceous glands are located throughout the whole body except the palms, soles, dorsa of the feet and lower lip. The sebaceous glands produce a complex mixture of oily material called sebum, which normally empties onto the skin surface through the opening of the follicle. Acne is believed to result from blockage of the follicle opening, which prevents the sebum from passing through. Most acne researchers believe that there are multiple factors involved in the development of acne lesions, such as increased sebum production, blockage of the pilosebaceous unit, bacterial colonization of the pilosebaceous unit, and inflammation.

Increased sebum production, which leads to a greasy appearance of the skin, is implicated in the development of acne. Sebum is a lipid-rich secretion from sebaceous glands and its production is directly dependent on the size and rate of growth of the sebaceous glands. Sebum production is under the control of androgenic hormones, and androgens have been indicated as a stimulus for enlargement of sebaceous glands and increased sebum production. The onset of acne is typically associated with hormonal surges before and during puberty.

Blockage of the pilosebaceous units have also been attributed as a contributing factor in the development of acne. Blockage of pilosebaceous units can be the result of proliferation of keratinocytes around the pilosebaceous duct (pore), thereby leading to blockage of the duct. Blackheads (open comedones) and whiteheads (closed comedones) result from such blockage.

Another contributing factor in the development of acne is bacterial colonization of the pilosebaceous units. The bacteria implicated in the pathogenesis of acne are gram positive and anaerobic. Due to their anaerobic nature, bacteria that have been identified thus far from acne cultures are those that have been successfully cultured under aerobic conditions; thus, it is possible that additional strains of bacteria may be implicated in the development of acne. Most of the bacteria identified to date belong to the genus Propionibacterium, such as P. acnes, P. avidum, and P. granulosum. To a lesser extent, Staphylococcus epidermis and Staphylococcus aureus have been identified and associated with acne. Sebum that is trapped in the follicular canal favors proliferation of P. acnes. P. acnes may play a role in converting comedonal acne to inflammatory acne by producing enzymatic and chemical agents that promote inflammation (e.g., lipases, proteases, hyaluronidase, and chemotactic factors). For example, recent research has reported a role for P. acnes in neutrophil attraction (Tucker et al., Journal of Investigative Dermatology, Vol. 89, p. 9-16, 1980; Bershad, supra.

Inflammation is yet another contributing factor in the development of acne. Inflammation typically results after an initial phase of non-inflammatory acne, where there is both abnormal shedding of follicular epithelium and proliferation of P. acnes. Trapped P. acnes interact with the contents of a closed comedo (whitehead) to create an inflammatory lesion. The lesions can be superficial (red papules plus superficial pustules) or deep (pustules, nodules and cysts), and very often lead to scarring if not treated adequately.

Other factors that are attributed to the development of acne include external physical factors such as friction (acne mechanica) or contact with irritant oils or cosmetics (acne cosmetica).

The clinical presentation of acne is divided into three categories, comedonal, papulopostular, and nodulocystic acne, wherein the latter category is the most severe. Comedonal acne is the earliest clinical expression of acne and usually involves non-inflammatory comedones that are typically found on the central forehead, chin, nose, and paranasal areas. This form of acne develops in the pre-teenage or early teenage years and can be brought about by increased sebum production and abnormal desquamation of epithelial cells. Colonization with P. acnes does not usually occur in this category, and thus inflammatory lesions are typically not present. After the initial phase of non-inflammatory comedonal acne (a mild form of acne with non-inflammatory lesions), a mild to moderate form of inflammatory acne, called papulopustular acne (with inflammatory lesions), can develop in which there are scattered small papules (e.g., less than 5mm in diameter) and pustules (e.g., with a visible central core of purulent material) with a minimum of comedones. Papulopustular acne tends to develop in adult women in their 20s and 30s. The latest stage, nodular or nodulocystic acne, is the most severe and persistent stage of acne, and is associated with large, deep inflammatory nodules or cysts (e.g., greater than 5 mm in diameter). Nodules may become suppurative or hemorrhagic. Suppurative nodular lesions have been referred to as cysts because of their resemblance to inflamed epidermal cysts. Recurring rupture and reepithelialization of cysts leads to epithelial-lined sinus tracks, often accompanied by disfiguring scars.

Currently, there are a number of prescription based and over-the-counter (OTC) therapy options available for the treatment of acne. Although there are a variety of treatment formulations, approximately half of all acne patients use at least one form of topical therapy, such as a solution, gel, cream, or lotion. Some commonly prescribed topical treatments include, for example, benzoyl peroxide, retinoids or retinoid derivatives, antimicrobial agents such as azelaic acid and such as antibiotics or antibiotic combinations including clindamycin, tetracycline, doxycycline, or erythromycin with or without benzoyl peroxide. Representative examples of active agents in some OTC treatments include benzoyl peroxide, resorcinol, sulfur, and salicylic acid.

Despite the plentitude of currently available treatment options, acne has been disappointingly resistant to both prescription based and over-the-counter treatment methods. Disadvantages in existing treatments include slow times to commence action, unfavorable side effect profiles, ineffective killing of bacterial organisms, and, in the case of long term antibiotic therapy, bacterial resistance.

For many therapies, there is a lead-in period of approximately 6-8 weeks before any treatment benefits may be noticed. Currently, patients do not see noticeable improvement until after several months of using benzoyl peroxide, topical retinoids, or topical antibiotics. This extended time period is unacceptable for many young adult patients and can lead to non-compliance. In addition, many patients exhibit an initial worsening of acne with current therapies. For example, initial treatment with retinoids commonly results in a flare up of acne that develops in the early weeks of treatment, due to an eruption of existing microcomedones. Moreover, maximum improvement may not be evident for 3-4 months (Leyden, J J., New England Journal of Medicine, Vol. 336, p. 1156-1162, 1997).

Unfavorable side effects, such as skin irritation, is common in many of the existing therapies. In addition, almost all acne products cause a certain degree of erythema (skin redness), dry skin, burning on application, and itching (especially with retinoids). Furthermore, many over-the-counter medications produce surface exfoliation without being keratolytic, and thus cause epidermal peeling without affecting the underlying pathological process. Moreover, in the case of retinoids, there is a risk of photosensitivity.

Development of bacterial resistance is a potential hazard with long term antibiotic therapy. Some reports have estimated that the incidence of antibiotic-resistant acne-associated Propionibacterium is over 50% (Eady, E A., Dermatology, Vol. 196, p. 59-66,1998; Toyoda, M., Morohashi, M., Dermatology, Vol. 196, p. 130-134, 1998; Espersen, F., British Journal of Dermatology, Vol. 139, p. 4-8, 1998). Resistance in P. acnes has been seen in Europe and the United States with increasing frequency. Studies reveal that resistance against erythromycin is most prevalent, with the majority of the strains also being resistant to clindamycin (Toyoda, M., Morohashi, M., Dermatology, Vol. 196, p. 130-134, 1998). Cross-resistance between tetracycline and doxycycline has also been reported. The development of bacterial resistance may have greater consequences than the simple failure of acne treatment. The spread of antibiotic resistance to Staphylococci has become a growing concern, because such an organism can have deleterious and sometimes fatal consequences in immunocompromised patients. In a worse case scenario, it has been predicted that within 5-10 years virtually all strains of P. acnes will be resistant to erythromycin, resulting in a consequential loss of clinical efficacy in erythromycin therapy (Eady, E A., Dermatology, Vol. 196, p. 59-66, 1998).

Therefore, there remains an unmet need for improved treatment options for acne that are effective, fast-acting, display a favorable side-effect profile, and/or do not develop bacterial resistance.

XMP.629 is a biologically active compound derived from functional domain II (amino acid residues 65-99) of human bactericidal/permeability-increasing protein (BPI). XMP.629 is a nanopeptide and has a net +4 charge at physiological pH. The corresponding free base has a molecular weight of 1283 Daltons. All of the amino acids in XMP.629 are D enantiomers. The C-termini of XMP.629 is amidated and the sequence is as follows:
NH₂-lys-leu-phe-arg-(3-(1-naphthyl)-ala)-gln-ala-lys-(3-(1-naphthyl)-ala)-CONH₂ (SEQ ID NO: 1).
   XMP.629 has a maximal UV absorption in aqueous solution at 282 nm.

XMP.629 has been previously described, for instance, in co-owned U.S. Pat. No. 6,515,104 and WO 01/00655 (PCT/US00/17358). XMP.629 and its properties have been additionally described in Lim, et al., “F-346: XMP.629, a Peptide Derived from Function Domain II of BPI, Demonstrates Broad-Spectrum Antimicrobial and Endotoxin-Neutralizing Properties In Vitro and In Vivo”, ICAAC 2001 Poster Presentation, 41^{st} Interscience Conference on Antimicrobial Agents and Chemotherapy, Chicago, Ill., Dec. 16-19, 2001. Properties and activities of XMP.629 include, for example, neutralizing heparin, inhibiting endothelial cell proliferation, and/or inhibiting angiogenesis. Additional properties and activities of XMP.629 include LPS binding, LPS neutralization and/or antimicrobial activity, such as anti-bacterial, anti-fungal or anti-protozoal. There are no prior disclosures that teach or suggest the treatment of acne with XMP.629.

### SUMMARY OF THE INVENTION

The present invention provides novel methods and materials for treating acne. The present invention provides methods for treating acne comprising administering to a subject a composition comprising a therapeutically effective amount of XMP.629 or a pharmaceutically acceptable salt or derivative thereof. A therapeutically effective amount includes an amount whereby the acne is ameliorated. Amelioration of the acne is indicated by an amelioration of one or more symptoms or signs of acne, including clinical symptoms or signs of acne, and is preferably indicated by a reduction in inflammatory lesion count, reduction in non-inflammatory lesion count, reduction in total lesion count, or an increased proportion of clear or almost clear skin. A therapeutically effective amount is preferably an amount that does not result in the development of bacterial resistance after repeated treatment.

The invention also provides methods for ameliorating acne comprising administering to a subject a composition comprising a therapeutically effective amount of XMP.629 or a pharmaceutically acceptable salt or derivative thereof, wherein the amelioration is indicated by at least one of the following: reduction in inflammatory lesion count; reduction in non-inflammatory lesion count; reduction in total lesion count; or an increased proportion of clear or almost clear skin. Inflammatory and/or non-inflammatory acne lesions can be open or closed comedones, papules, pustules, or nodules appearing on and/or in the subject's skin. Areas of the subject's skin which may present inflammatory and/or non-inflammatory acne lesions include, for example, the face, upper back, and chest. Areas of the subject's skin which are void (or nearly void) of inflammatory and/or non-inflammatory acne lesions are considered to be clear or almost clear.

The invention also provides novel compositions, including pharmaceutical compositions and formulations comprising XMP.629 or a pharmaceutically acceptable salt or derivative thereof. The invention provides compositions comprising XMP.629 or a pharmaceutically acceptable salt or derivative thereof and further comprising one or more of the following: a poloxamer surfactant(s), EDTA, benzalkonium chloride, propylene glycol and/or hydroxyethylcellulose.

The invention also provides methods for treating acne comprising concurrently administering to a subject with acne (i) a composition comprising a therapeutically effective amount of XMP.629 or a pharmaceutically acceptable salt or derivative thereof and (ii) at least one anti-acne agent. The anti-acne agent is not XMP.629 or pharmaceutically acceptable salt or derivative thereof. The acne is ameliorated by concurrent administration of (i) and (ii). The anti-acne agent may be a prescription based or over-the-counter agent. Exemplary anti-acne agents include benzoyl peroxide, retinoids, retinoid derivatives, antimicrobial agents, or combinations thereof.

The invention also provides methods of cosmetically treating a subject. The methods comprise administering to a subject XMP.629 or a physiologically acceptable salt or derivative thereof, including cosmetic compositions and formulations comprising XMP.629 or a physiologically acceptable salt or derivative thereof. A cosmetically effective amount includes an amount whereby the subject's skin is cosmetically treated. A cosmetically effective amount is preferably an amount effective for cosmetically improving the clarity of skin and/or for decreasing redness of skin.

The invention also provides methods for reducing or reversing resistance or development of resistance of an acne-associated bacterium to at least one anti-acne agent. The methods comprise administering to a subject with acne a composition comprising a therapeutically effective amount of XMP.629 or a pharmaceutically acceptable salt or derivative thereof, wherein the anti-acne agent is not XMP.629 or a physiologically or pharmaceutically acceptable salt or derivative thereof.

The invention also provides creams, gels, lotions, solutions, patches, impregnated dressings, gel sticks, sprays, aerosols, swabs, and wipes comprising XMP.629 or a physiologically or pharmaceutically acceptable salt or derivative thereof. The creams, gels, lotions, solutions, patches, impregnated dressings, gel sticks, sprays, aerosols, swabs, and wipes optionally also include least one anti-acne agent, wherein the anti-acne agent is not XMP.629 or a physiologically or pharmaceutically acceptable salt or derivative thereof.

The invention also provides kits comprising (i) XMP.629 or a physiologically or pharmaceutically acceptable salt or derivative thereof and (ii) at least one anti-acne agent, wherein the anti-acne agent is not XMP.629 or a physiologically or pharmaceutically acceptable salt or derivative thereof, for sequential or simultaneous administration to a subject in a method of ameliorating or treating acne.

The invention also provides articles containing XMP.629 or a physiologically or pharmaceutically acceptable salt or derivative thereof alone or in combination with at least one anti-acne agent, wherein the anti-acne agent is not XMP.629 or a physiologically or pharmaceutically salt or derivative thereof, for sequential or simultaneous administration to a subject in a method of ameliorating or treating acne.

The invention also provides methods comprising the step of selecting a subject with acne, including a subject experiencing resistance or development of resistance of an acne-associated bacterium to at least one anti-acne agent, and the step of administering XMP.629 or a physiologically or pharmaceutically acceptable salt or derivative thereof alone or in combination with at least one anti-acne agent, wherein the anti-acne agent is not XMP.629 or a physiologically or pharmaceutically salt or derivative thereof.

The foregoing methods and materials preferably include compositions for repeated administration.

### DETAILED DESCRIPTION OF THE INVENTION

The present invention provides novel methods and materials including for treating acne. The present invention provides methods of treating acne comprising administering to a subject, including a patient in need thereof, therapeutically effective amounts of XMP.629 or a pharmaceutically acceptable salt or derivative thereof. Treating or treatment includes prophylactic and/or therapeutic treatment. Therapeutically effective amounts include amounts that ameliorate the acne. Amelioration of acne is indicated by an attenuation (e.g., decrease, reduction or removal) of one or more of the symptoms or signs of acne, including clinical symptoms or signs of acne, and is preferably indicated by at least one of the following: reduction in inflammatory lesion count, reduction in non-inflammatory lesion count, reduction in total lesion count, or an increased proportion of clear or almost clear skin. Therapeutically effective amounts are preferably amounts that do not induce bacterial resistance after repeated administration of XMP.629 or a pharmaceutically acceptable salt or derivative thereof.

Without being bound by a theory of the invention, XMP.629 or a pharmaceutically acceptable salt or derivative thereof may exert its surprising and beneficial effects by one or more mechanisms involved in the pathogenesis of acne, including but not limited to antimicrobial (e.g. antibacterial and/or antifungal including with resistant organisms) mechanisms or activities, anti-inflammatory mechanisms or activities, keratinolytic mechanisms or activities, and/or reduction of sebum production or differentiation of the sebum gland mechanisms or activities.

Methods and materials of the present invention are drawn to XMP.629, pharmaceutically acceptable salts of XMP.629 and derivatives of XMP.629. XMP.629 derivatives are compounds that have altered amino acid sequences, for example, by substitutions, additions, or deletions, wherein the altered amino acid sequence still provides for functionally equivalent molecules, or for functionally enhanced molecules, as desired. XMP.629 derivatives include, but are not limited to, those containing altered sequences in which functionally equivalent amino acid residues are substituted for residues within the XMP.629 sequence. For example, one or more amino acid residues within the XMP.629 sequence can be substituted by another amino acid of a similar polarity which acts as a functional equivalent. Substitutions for an amino acid within the sequence may be selected from other members of the class to which the amino acid belongs. For example, a nonpolar amino acid can be replaced with another nonpolar (hydrophobic) amino acid such as alanine, leucine, isoleucine, valine, proline, phenylalanine, tryptophan, and methionine. Another example is when a polar neutral amino acid is substituted with another polar neutral amino acid, such as glycine, serine, threonine, cysteine, tyrosine, asparagine, and glutamine. Likewise, a basic amino acid can be replaced with a positively charged (basic) amino acid, such as arginine, lysine, and histidine, and an acidic amino acid can be replaced with a negatively charged (acidic) amino acid, such as aspartic acid and glutamic acid. The above referenced substitutions are generally understood to be conservative substitutions.

XMP.629 derivatives include small peptide-based constructs as described in U.S. Pat. No. 6,515,104 and International Publication No. WO 01/00655 (PCT/US00/17358), which are incorporated herein by reference. Such constructs are 8-14 amino acid moieties in length, having a sequence that is derived from or based on reverse subsequences identified and selected from functional domain 11 (amino acids 65-99) of bactericidal/ permeability-increasing protein (BPI), as described in U.S. Pat. No. 6,515,104 and International Publication No. WO 01/00655. Such reverse subsequences consist of a minimum core sequence based on an amino acid motif derived from amino acids 99-92 of BPI. Reverse subsequences include substituted subsequences (for example, amino acids 99-92, 99-91, 99-90, 99-89, 99-88, 99-87, 99-86, or 99-85 wherein the substitutions are at 95 and 91). Such sequences preferably have one or more D-amino acid moieties and most preferably have each or all of the amino acid moieties are D isomers.

XMP.629 derivatives include the following sequences:

| | | |
|---|---|---|
| XMP.624 | k-l-f-r-(naph-a)-q-a-k-(naph-a)-k-g-s-i-k | (SEQ ID NO: 2) |
| XMP.625 | k-l-f-r-(naph-a)-q-a-k-(naph-a)-k-g-s-i | (SEQ ID NO: 3) |
| XMP.626 | k-l-f-r-(naph-a)-q-a-k-(naph-a)-k-g-s | (SEQ ID NO: 4) |
| XMP.627 | k-l-f-r-(naph-a)-q-a-k-(naph-a)-k-g | (SEQ ID NO: 5) |
| XMP.628 | k-l-f-r-(naph-a)-q-a-k-(naph-a)-k | (SEQ ID NO: 6) |
| XMP.630 | k-l-f-r-(naph-a)-q-a-k | (SEQ ID NO: 7) |
| XMP.656 | k-l-f-r-(naph-a)-q-a-k-(naph-a)-k-g-i-k-i | (SEQ ID NO: 8) |
| XMP.679 | k-l-f-k-(naph-a)-q-a-k-(naph-a)-k-g | (SEQ ID NO: 9) |
| XMP.684 | (biphenyl-A)-k-l-f-r-(naph-a)-q-a-k | (SEQ ID NO: 10) |
| XMP.685 | k-l-f-r-(biphenyl-A)-q-a-k | (SEQ ID NO: 11) |
| XMP.725 | k-l-f-r-(biphenyl-a)-q-a-k | (SEQ ID NO: 12) |
| XMP.728 | k-l-f-k-(biphenyl-a)-q-a-k-(biphenyl-a)-k-G | (SEQ ID NO: 13) |
| XMP.760 | k-a-f-r-(naph-a)-q-a-k-(naph-a) | (SEQ ID NO: 14) |
| XMP.764 | k-a-f-k-(naph-a)-q-a-k-(naph-a)-k-G | (SEQ ID NO: 15) |
| XMP.776 | k-l-f-k-(naph-a)-q-a-k-(naph-a) | (SEQ ID NO: 16) |
| XMP.778 | k-(aminoisobutyric acid)-f-r-(naph-a)-q-a-k-(naph-a) | (SEQ ID NO: 17) |
| XMP.661 | 2-biphenylcarbonyl-k-l-f-r-(naph-a)-q-a-k | (SEQ ID NO: 18) |
| XMP.664 | 4-biphenylcarbonyl-k-l-f-r-(naph-a)-q-a-k | (SEQ ID NO: 19) |
| XMP.666 | 2-naphthylacetyl-k-l-f-r-(naph-a)-q-a-k | (SEQ ID NO: 20) |
| XMP.671 | 1 -naphthylacetyl-k-l-f-r-(naph-a)-q-a-k | (SEQ ID NO: 21) |
| XMP.699 | 2-biphenylenecarbonyl-k-l-f-r-(naph-a)-q-a-k | (SEQ ID NO: 22) |
| XMP.767 | 4-biphenylcarbonyl-k-l-f-k-(naph-a)-q-a-k | (SEQ ID NO: 23) |
| XMP.768 | 4-biphenylcarbonyl-k-l-f-r-(biphenyl-a)-q-a-k | (SEQ ID NO: 24) |
| XMP.769 | 4-biphenylcarbonyl-k-l-f-k-(biphenyl-a)-q-a-k | (SEQ ID NO: 25) |

Compositions comprising XMP.629 presented herein may encompass XMP.629 derivatives that comprise a conservative substitution wherein the substituted amino acid is a non-natural amino acid residue or an amino acid analog and provided that the XMP.629 derivative retains the desired functional activity. Examples of non-naturally occurring or derivatized non-naturally occurring amino acids include N-α-methyl amino acids, C-α-methyl amino acids, β-methyl amino acids, β-alanine (β-Ala), norvaline (Nva), norleucine (Nle), 4-aminobutyric acid (γ-Abu), 2-aminoisobutyric acid (Aib), 6-aminohexanoic acid (ε-Ahx), ornithine (orn), hydroxyproline (Hyp), sarcosine, citrulline, cysteic acid, cyclohexylalanine, α-amino isobutyric acid, t-butylglycine, t-butylalanine, and phenylglycine.

A derivative of XMP.629 includes, but is not limited to, a derivative comprising additional chemical moieties not normally a part of the peptide, provided that the derivative retains the desired functional activity of the peptide. Examples of such derivatives include: (a) N-acyl derivatives of the amino terminal or of another free amino group, wherein the acyl group may be either an alkanoyl group, e.g., acetyl, hexanoyl, octanoyl, an aroyl group, e.g., benzoyl, or a blocking group such as Fmoc (fluorenylmethyl-O—CO—), carbobenzoxy (benzyl-O—CO—), monomethoxysuccinyl, naphthyl-NH—CO—, acetylamino-caproyl, adamantyl-NH—CO—; (b) esters of the carboxyl terminal or of another free carboxyl or hydroxy groups; and (c) amides of the carboxyl terminal or of another free carboxyl groups produced by reaction with ammonia or with a suitable amine.

Also included among the chemical derivatives are those derivatives obtained by modification of the peptide bond —CO—NH—, for example, by: (a) reduction to —CH₂—NH—; (b) alkylation to —CO—N(alkyl)-; and (c) inversion to —NH—CO—.

XMP.629 and pharmaceutically acceptable salts and derivatives of XMP.629 can be prepared by a variety of well-known chemical procedures. XMP.629 and salts or derivatives thereof can be prepared by any synthetic means available to one skilled in the art. The precise method employed for synthesizing XMP.629 and salts or derivatives thereof is not to be considered as limiting, particularly as technology develops additional ways to synthesize and assemble amino acids and/or amino acid derivatives, including naturally or non-naturally occurring D and/or L amino acids. Standard methods can be used to synthesize XMP.629 and pharmaceutically acceptable salts or derivatives thereof.

A standard method for preparing XMP.629 or pharmaceutically acceptable salts or derivatives thereof is solution phase synthesis. A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for the preparation of XMP.629 or pharmaceutically acceptable salts or derivatives thereof. The nature and implementation of modifications to these devices (if any) so that they can operate as discussed herein will be apparent to persons skilled in the chemical or synthetic arts.

Another standard method for peptide synthesis is solid phase synthesis, and automated equipment for systematically constructing peptide chains can be employed. For example, XMP.629 or pharmaceutically acceptable salts or derivatives thereof can be prepared by solid phase peptide synthesis as described in co-assigned U.S. patent application Ser. No. 08/183,222, abandoned, and U.S. Pat. No. 5,733,872, according to the methods of Merrifield, J. Am Chem. Soc., Vol. 85, p. 2149, 1963 and Merrifield et al., Anal. Chem., Vol. 38, p. 1905-1914, 1966 using an automated peptide synthesizer.

As described herein, XMP.629 was synthesized using a modified solid-phase procedure first described by Merrifield. For example, XMP.629 or its derivatives can be obtained by solid phase peptide synthesis which, in brief, involves coupling the carboxyl group of the C-terminal amino acid to a resin and successively adding N-a protected amino acids. The protecting groups may be any such groups known in the art. Thus, an XMP.629 derivative can comprise fully protected or partially protected XMP.629, wherein XMP.629 comprises at least one protecting group. Before each new amino acid is added to the growing chain, the protecting group of the previous amino acid added to the chain is removed. The coupling of amino acids to appropriate resins has been described, for example, by Rivier et al. (U.S. Pat. No. 4,244,946). Such solid phase syntheses have been described, for example, by Merrifield, 1964, J. Am. Chem. Soc. 85, 2149; Vale et al. 1981, Science 213, 1394-1397; Marki et al., 1981, J. Am. Chem. Soc. 103, 3178, and in U.S. Pat. Nos. 4,305,872 and 4,316,891. In a preferred aspect, an automated peptide synthesizer is employed.

In the synthesis of peptides, D-amino acids or protected D-amino acids can be utilized rather than or in addition to the conventional L-amino acids. D-amino acids suitable for polypeptide synthesis are commercially available from, for example, the Peptide Institute (Osaka, Japan), Peptides International (Louisville, Ky.), Bachem Bioscience (Philadelphia, Pa.), Bachem Calif., (Torrance, Calif.), and PolyPeptide Labs (Torrance, Calif.). The method of synthesizing a D-polypeptide is analogous to the method of synthesizing a L-polypeptide. For example, in a solid phase synthesis, the protected or derivatized D-amino acid is attached to an inert solid support through its unprotected carboxyl or amino group. The protecting group of the amino or carboxyl group is then selectively removed and the next D-amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added D-amino acid residue, and the next D-amino acid (suitably protected) is then added, and so forth. After all the desired D-amino acids have been linked in the proper sequence, any remaining terminal and side group protecting groups (and solid support) can be removed sequentially or concurrently, to afford the synthetic polypeptide. XMP.629 or pharmaceutically acceptable salts or derivatives thereof may comprise at least one protecting group, such as in the case of a fully protected or partially protected XMP.629 compound.

Purification of the synthesized peptides or peptide derivatives is carried out by well known standard methods, including chromatography (e.g., ion exchange, affinity, sizing column chromatography, and reverse-phase HPLC (high performance liquid chromatography, including analytical RF-HPLC), centrifugation, differential solubility, hydrophobicity, mass spectral analysis, amino acid analysis, or by any other standard technique for purification of peptides. In an embodiment, mass spectral analysis is employed. In another embodiment, reverse phase HPLC is employed. In another embodiment, amino acid analysis is employed.

XMP.629 derivatives, and other small molecules, such as peptidomimetics, may also be used to prepare analogous molecular structures having similar properties to XMP.629. Thus, the invention is contemplated to include molecules in addition to those expressly disclosed that share the structure, hydrophobicity, charge characteristics, and side chain properties of the specific embodiments exemplified herein.

XMP.629 can be present in the instant compositions as a free base, a physiologically or pharmaceutically acceptable salt, or a combination thereof. The physiologically or pharmaceutically acceptable salt embraces an inorganic or an organic salt. Representative salts include hydrobromide, hydrochloride, mucate, succinate, n-oxide, sulfate, malonate, acetate, phosphate dibasic, phosphate monobasic, acetate trihydrate, bi(heplafluorobutyrate), maleate, bi(methylcarbamate), bi(pentafluoropropionate), mesylate, bi-(pyridine-3-carboxylate), bi(trifluoroacetate), bitartrate, chlorhydrate, fumarate and sulfate pentahydrate. A preferred physiologically or pharmaceutically acceptable salt of XMP.629 is acetate.

XMP.629 or a pharmaceutically acceptable salt or derivative thereof may be administered topically (e.g., in doses from about 0.001% to about 10% or preferably from about 0.001% to about 1% or from about 0.005% to about 0.5%, weight to volume or weight to weight) or systemically (e.g., in doses from about 1 μg/kg to about 100 mg/kg per day, preferably from about 0.1 mg/kg to about 20 mg/kg per day. Systemic routes of administration include, for example, oral or transdermal. For the treatment of acne, topical administration is preferred.

In a preferred embodiment, compositions comprising XMP.629 or a pharmaceutically acceptable salt or derivative thereof is in any cosmetic or physiologically acceptable form which is generally used for topical application, such as liquids (both aqueous and non-aqueous solutions), gels (both aqueous and non-aqueous), lotions, serums, ointments, paste, powders, liposomes, laminates, microspheres, patches, capsules, and tablets, and creams (both oil-in-water and water-in-oil emulsions). Topical formulations may be presented as, for instance, ointments, creams or lotions, impregnated dressings, patches, gels, gel sticks, sprays, aerosols, wipes, and swabs. Preferable topical formulations include gels, creams, solutions and lotions.

In the topical treatment of acne, the formulation vehicle of the active agent is an additional consideration. A variety of suitable formulation vehicles can be utilized, and some vehicles may be preferable for different skin types. For example, creams may be more appropriate for patients with sensitive or dry skin who may prefer a nonirritating and nondrying formulation, whereas patients with oily skin may complain of an “oily” feel with creams. Patients who have oily skin may prefer gels, which tend to have a drying effect. However, some gels can cause a burning-type irritation in some patients and can also prevent certain kinds of cosmetics from adhering to the skin. Lotions can be suitable with a variety of skin types, and tend to spread well over hair-bearing skin. However, lotions often contain propylene glycol and can have burning or drying effects. Solutions are often used with topical antimicrobial agents such as antibiotics, which are often dissolved in alcohol. Like gels, solutions may be preferred by patients with oily skin.

Compositions useful in methods of the present invention may also contain additives such as water, alcohols, oils (mineral vegetable, animal and synthetics), glycols, colorants, preservatives, emulsifiers, gelling agents, gums, esters, hormones, steroids, antioxidants, silicones, polymers, fragrances, flavors, sunscreens, other active ingredients, acids, bases, buffers, vitamins, minerals, salts, polyols, proteins and their derivative essential oils, other enzymes, co-enzymes and extracts, surfactants, detergents, soaps, anionics, non-ionics, ionics, waxes, lipids, UV filters, stabilizers, fillers, celluloses, glycans, amines, solubilizers, thickeners, sugars and sugar derivatives, ceramides, sweeteners, and the like. The formulations may contain compatible conventional carriers, such as cream or ointment bases, and ethanol or oleyl alcohol for lotions.

Preferably, a variety of agents, including chelating or complexing agents, tonicity agents, gelling agents, buffers, surfactants, preservatives, and/or solvents may be added in a variety of concentrations to the present compounds or compositions. For example, chelating or complexing agents, including, for example, EDTA disodium, dihydrate, can be added to the present compounds or compositions. Solvents and/or agents that may facilitate skin penetration, including for example, propylene glycol, can be added to the present compounds or compositions. Additionally, agents that may act as preservatives, including, for example, benzalkonium chloride, can be added to the present compounds or compositions including concentration that is not effective as a preservative. Surfactants, including, for example, poloxamar surfactants such as poloxamer 333, poloxamer 334, poloxamer 335 or poloxamer 403 (e.g., BASF Pluronic P-103, P-104, P-105, or P-123, respectfully), can be added to the present compounds or compositions. Further, agents that may act as gelling agents, such as hydroxyethylcellulose (e.g., 250 HHX) can be added to the present compounds or compositions.

A variety of suitable creams, lotions, gels, sticks, ointments, sprays, patches, swabs, wipes or aerosol formulations are well known in the art, are useful for the formulation of XMP.629 or a pharmaceutically acceptable salt or derivative thereof, and are described, for example, in standard text books of pharmaceutics and cosmetics, such as Harry's Cosmetology published by Leonard Hill Books, Remington's Pharmaceutical Sciences and The British and U.S. Pharmacopoeias, the disclosures of which are incorporated herein by reference. In addition, novel compositions of XMP.629 or a pharmaceutically acceptable salt or derivative thereof can include one or more of the following agents: a poloxamer surfactant(s) (see, e.g., U.S. Pat. Nos. 5,448,034 or 5,912,228), EDTA, benzalkonium chloride (BAK), hydroxyethylcellulose (HEC) and/or propylene glycol (PG). [001] For example, novel compositions of XMP.629 or a pharmaceutically acceptable salt or derivative thereof can include the following combinations of agents: poloxamer surfactant and EDTA; or poloxamer surfactant and BAK; or poloxamer surfactant and propylene glycol; or poloxamer surfactant and hydroxyethylcellulose; or EDTA and BAK; or EDTA and propylene glycol; or EDTA and hydroxyethylcellulose; or BAK and propylene glycol; or BAK and hydroxyethylcellulose; or propylene glycol and hydroxyethylcellulose; or poloxamer surfactant, EDTA and BAK; or poloxamer surfactant, EDTA and propylene glycol; or poloxamer surfactant, EDTA and hydroxyethylcellulose; or poloxamer surfactant, BAK and propylene glycol; or poloxamer surfactant, BAK and hydroxyethylcellulose; or poloxamer surfactant, propylene glycol, and hydroxyethylcellulose; or EDTA, BAK and propylene glycol; or EDTA, BAK and hydroxyethylcellulose; or BAK, propylene glycol and hydroxyethylcellulose; or poloxamer surfactant, EDTA, BAK and propylene glycol; or poloxamer surfactant, EDTA, BAK and hydroxyethylcellulose; or EDTA, BAK, propylene glycol and hydroxyethylcellulose; or poloxamer surfactant, EDTA, BAK, propylene glycol and hydroxyethylcellulose.

Compositions comprising XMP.629 or a pharmaceutically acceptable salt or derivative thereof can be administered to a subject in a variety of doses and formulations and as many times as needed to effect amelioration of the acne. Amelioration of acne in a subject can be indicated by any well known criteria in the dermatological art, including, for example, by attenuating (e.g., decreasing, reducing or removing) one or more symptoms or signs of acne such as clinical symptoms or signs of acne. Preferably, amelioration of the acne is indicated by at least one of the following: reduction in inflammatory lesion count, reduction in non-inflammatory lesion count, reduction in total lesion count, or an increased proportion of clear or almost clear skin. Inflammatory and/or non-inflammatory acne lesions can be open or closed comedones, papules, pustules, or nodules appearing on and/or in the subject's skin. Areas of the subject's skin which may present inflammatory and/or non-inflammatory acne lesions include, for example, the face, upper back, and chest. Areas of the subject's skin which are void (or nearly void) of inflammatory and/or non-inflammatory acne lesions are considered to be clear or almost clear. Administration of the compositions presented herein may include multiple administrations per day (e.g., from about one to about 12 administrations per day). In a preferred embodiment, administration of the compositions presented herein is performed from about once to about five times a day. In another preferred embodiment, administration of the compositions presented herein is performed from about three to about four times a day. In yet another preferred embodiment, administration of the compositions presented herein is performed about once a day.

According to the methods of treatment of the present invention, acne is treated (e.g., therapeutically or prophylactically) in a subject, for example, in a mammal, such as a human, by administering a composition comprising a therapeutically effective amount of XMP.629 or a pharmaceutically acceptable salt or derivative thereof, in such amounts and for such time as is necessary to achieve the desired result. Therapeutically effective amounts of XMP.629 or a pharmaceutically acceptable salt or derivative thereof, include sufficient amounts to treat acne, at a reasonable benefit/risk ratio applicable to any medical treatment. It will be understood, however, that the total daily usage and compositions of the present invention will be decided by the attending physician within the scope of sound medical judgment. The specific therapeutically effective dose level for any particular subject will depend upon a variety of factors including the stage of the disorder being treated and the severity of the disorder, the activity of the specific compound employed, the specific compound or composition employed, the age, body weight, general health, sex, and diet of the subject, the time of administration, route of administration, and rate of excretion of the specific compound employed, the duration of the treatment, drugs used in combination or coincidental with the specific compound employed, and like factors well known in the medical arts.

In a preferred embodiment, the therapeutically effective amount of XMP.629 or a pharmaceutically acceptable salt or derivative thereof ranges from about 0.005% to about 0.5% (weight to volume). In another preferred embodiment, the therapeutically effective amount of XMP.629 or a pharmaceutically acceptable salt or derivative thereof ranges from about 0.01% to about 0.2% (weight to volume). In yet another preferred embodiment, the therapeutically effective amount of XMP.629 or a pharmaceutically acceptable salt or derivative thereof ranges from about 0.01% to about 0.1% (weight to volume). In yet another preferred embodiment, the therapeutically effective amount of XMP.629 or a pharmaceutically acceptable salt or derivative thereof ranges from about 0.05% to about 0.1% (weight to volume). In a preferred composition, the therapeutically effective amount of XMP.629 or a pharmaceutically acceptable salt or derivative thereof is about 0.01% (weight to volume). In another preferred composition, the therapeutically effective amount of XMP.629 or a pharmaceutically acceptable salt or derivative thereof is about 0.05% (weight to volume). In yet another preferred composition, the therapeutically effective amount of XMP.629 or a pharmaceutically acceptable salt or derivative thereof is about 0.1% (weight to volume).

An anti-acne agent may refer to any agent that is known to be useful and/or effective in the treatment of acne. Anti-acne agents are capable of ameliorating acne, for example, by attenuating (e.g., decreasing, reducing, or removing) one or more symptoms or signs of acne, including clinical symptoms or signs of acne. Anti-acne agents include, for example, prescription based and/or over-the-counter (OTC) treatments.

Currently, there are a number of prescription based anti-acne agents available for the treatment of acne. Prescription based anti-acne agents are aimed at reducing several factors that contribute to the development of acne, such as abnormal clumping of cells in the follicles, increased oil production, bacterial colonization, and inflammation. The dermatologist choice of therapy for an individual patient depends on the extent, severity, duration, and the type of acne lesions exhibited by the patient. Although there exists a variety of treatment formulations, approximately half of all acne patients use at least one form of topical therapy. Some commonly prescribed topical and oral anti-acne treatments are described below.

Benzoyl peroxide is the most common first-line treatment for acne, particularly in comedonal acne. It is mainly bactericidal (e.g., for inflammatory acne consisting of papules, pustules and nodules/cysts) and, to a degree, comedolytic (Lever Marks R., Drugs, Vol. 39 (5), p. 681; O'Loughlin, S., Irish Medical Journal, Vol. 90, p. 3, 1997). Benzoyl peroxide is available in a variety of concentrations (1, 2.5, 5, and 10%) and formulations (solution, gel, and lotion); however, gels appear to be more effective vehicles than creams or oil-based lotions in releasing the benzoyl peroxide, but gels can also cause more irritation (Lever Marks R., Drugs, Vol. 39 (5), p. 681-692, 1990). The frequency and dose of benzoyl peroxide can be increased as tolerability to the agent develops. The most frequent adverse effect of benzoyl peroxide are irritant reactions, such as mild redness and skin peeling (Leyden, New England Journal of Medicine, Vol. 336, p. 1156-1162, 1997). Benzyol peroxide also bleaches hair and clothing (Lever Marks R., Drugs, Vol. 39 (5), p. 681-692,1990).

Retinoids and retinoid derivatives, such as retinol, retinal, tretinoin, isotretinoin, adapalene (6-[3-(1-adamantyl)-4-methoxyphenyl]-2-naphthoic acid), and tazarotene, and the like, are commonly used for the treatment of comedonal acne. Adapalene and tazarotene are recently developed topical polyaromatic retinoids and may provide therapeutic advantages over tretinoin. Retinoids bind to nuclear retinoic acid receptors upon cellular uptake and alter certain steps of gene transcription, resulting in changes of metabolic pathways, such as proliferation, differentiation, inflammation, and sebum production (Gollnick, H., et al., Dermatology, Vol. 196, p. 119-125, 1998). It has been suggested that activation of nuclear retinoic acid receptors affect keratinocyte differentiation and block inflammation (Bershad, S. V., The Mount Sinai Journal of Medicine, Vol. 68, p. 279-286, 2001).

Oral isotretinoin (13-cis retinoic acid) is marketed as Accutane® in 10, 20 and 40 mg capsules. This oral retinoid related to Vitamin A is not suitable for all types of acne but is used for control of acne and in the induction of long-term remissions. It has been reported to dramatically reduce sebum excretion, follicular keratinization, and ductal and surface P. acnes counts. A number of side effects occur during treatment such as mucous/skin effects, elevated triglyceride levels, musculoskeletal effects, headaches, elevated liver enzyme levels, amenorrhea and specifically including, for example, cheilitis, dry skin, pruritis, dry mouth, dry nose, epistaxis, conjunctivitis, musculoskeletal symptoms, rash, hair thinning and peeling. Isotretinoin is a potent teratogen and pregnancy must be avoided during treatment. Accutane® treatment has been associated with several reports of severe depression and suicide, however, Accutane® therapy has been indicated for the treatment of severely depressed or dysmorphophobic patients with acne.

Corticosteroids, such as prednisone, have a limited but definite place in the management of acne, particularly nodulocystic acne. Anti-inflammatory oral agents such as prednisone may be useful for rapidly advancing disease, including during the time that cysts may be improving slowly with other therapies such as with isotretinoin. Intralesional corticosteroid injections, such as with triamcinolone (e.g., Kenalog® 10 mg/ml or TAC-3® 3 mg/ml), may also be used. Prolonged, continual use of intralesional steroids has resulted in adrenal suppression.

Additional acne therapies include systemic treatment with hormone manipulation (e.g., estrogens, and/or progestins, glucocorticoids, or antiandrogens, including, spironolactone or flutamide) as well as acne surgery (e.g., manual removal of comedones and/or the drainage of pustules or cysts, scar revision, dermabrasion, scar excision or collagen implants).

Topical tretinoin (Retin-A®, Avita™), also known as retinoic acid or Vitamin A acid, helps open pores, and is able to loosen and remove comedones. Treatment with Retin-A® is usually reserved for moderate to severe acne because it is usually too drying for mild cases. However, it may be an agent of choice for noninflammatory acne consisting of open and closed comedones. During the initial 4 to 6 weeks of therapy, it is common to see redness and scaliness of the skin. Tretinoin provides a strong anticomedogenic and comedolytic effect and also possesses an indirect antimicrobial effect (O'Loughlin, S., Irish Medical Journal, Vol. 90, p. 3, 1997). However, it has very little anti-inflammatory efficacy and is not considered to be sebosuppressive. Tretinoin is approved for use in comedonal acne and mild forms of papulopustulosa acne. Tretinoin is typically applied once daily, starting with a lower concentration of the cream (available in 0.025, 0.05, and 0.1% concentrations), gel (0.01 and 0.025%), or microemulsion gel (0.1%). Tretinoin solution (0.05%) is the strongest and most irritating because tretinoin is available in different formulations, the physician can tailor treatment according to the sensitivity of the patient's skin and the patient's environment. For instance, gels are preferred in hot and humid climates whereas creams are more suitable for cold and dry climates. High concentrations of tretinoin may be applied to the skin without causing systemic toxicity because tretinoin remains mostly in the stratum corneum of the epidermis, and, when absorbed, is rapidly metabolized by the liver (Lever L., Marks R., Drugs, Vol. 39 (5), p. 681-692, 1990). Currently, there are a number of formulations which utilize lower tretinoin concentration (0.025%) with cream vehicles that are designed to be more emollient and less penetrating. For example, the Retin-A® Micro (tretinoin gel, 0.1%) microsphere formulation is designed as a slow delivery of tretinoin gel, in which the active ingredient is incorporated into microsponges, which are macroporous beads (10-25 microns). Despite these improvements however, the use of tretinoin in dermatology practice is often limited by most, if not all, patients developing a low-grade irritant dermatitis with redness and scaling. Tretinoin also increases sun sensitivity. In addition, the onset of action of tretinoin is relatively delayed and variable, with most patients seeing results in 1-3 months. Some patients experience clinical worsening after 2-4 weeks of treatment when the extrusion of comedones can elicit a pustular reaction. Tretinoin may be used in combination with other topical agents such as topical antibiotics and/or benzoyl peroxide, and may enhance their penetration.

Tazarotene cream (Tazorac™) is a synthetic acetylenic retinoid and has been approved by the FDA for topical treatment of acne. In addition, tazarotene gel (0.1%) has also been approved for mild-to-moderate acne vulgaris. Tazarotene is converted to a carboxylic acid active form by de-esterification. The converted form binds all three members of the retinoic acid receptor family and modifies gene expression. Although the mechanism of action is not well understood, tazarotene has been shown to suppress markers of inflammation in cultured epithelial cells. Tazarotene is typically applied once daily over the entire affected area. Adverse events reported with some patents with tazarotene cream for the treatment of acne included desquamation, dry skin, erythema, and burning sensation, as well as pruritus, irritation, face pain, and stinging.

Adapelene (Differin™) is a synthetic naphthoic acid derivative with retinoid activity. Adapelene has topical benefits similar to those of topical tretinoin and causes less local irritation. Its mechanism of action is twofold: first, it inhibits comedo formation through its ability to bind retinoic acid receptors and subsequently modulate cell differentiation; and second, it possesses direct anti-inflammatory activity. Adapelene is available as a gel or cream (0.1% concentration). It is initially applied 2-3 times per week, and usage is increased to nightly application over a period of about 2 months. Adapalene, like other retinoids, can cause an initial inflammatory flare-up toward the end of the first month of therapy. Erythema, scaling, dryness, persistent pruritus, and persistent burning and/or stinging has been reported with usage of adapalene in controlled clinical studies.

Azelaic acid is a dicarboxylic acid that has both comedolytic and anti-inflammatory effects. Azelaic acid has been shown to possess antimicrobial activity against P. acnes and Staphylococcus epidermidis. The antimicrobial action may be attributable to inhibition of microbial cellular protein synthesis. It is less potent than tretinoin but is useful in patients who cannot tolerate topical tretinoin or other retinoids. Topical azelaic acid has similar efficacy to topical benzoyl peroxide gel (5%), tretinoin cream (0.05%), erythromycin cream (2%), and oral tetracycline (0.5 to 1 g/day), in the treatment of comedonal and mild to moderate inflammatory acne. Azelaic acid is associated with a low rate of adverse effects, the most common being local itching and burning sensations. In clinical trials with azelaic acid, approximately 1-5% of patients reported pruritus (itching), burning, stinging, and tingling. There is also potential for allergic reactions with its usage.

Topical antimicrobial agents such as antibiotics are thought to be effective in the treatment of acne by killing or inhibiting P. acnes and are used for the treatment of mild forms of papulopustulosa acne. Representative examples of topical antibiotics include clindamycin (e.g. lincomycins), zithromycin, erythromycin, minocycline, and tetracycline. The first use of a topical antibiotic, erythromycin, for the treatment of acne was reported by Fulton (Fulton, J. E. Jr. and Pablo, G. Topical antibacterial therapy for acne. Study of the family of erythomycins. Arch. Dermatol. 110:83-86, 1974). Topical antibiotics are believed to work by decreasing the follicular population of P. acnes, as well as reducing the ability of this organism to generate pro-inflammatory molecules (Leyden, J J., American Journal of Clinical Dermatology, Vol. 2(4), p. 263-266, 2001). This results in a significant decrease in free fatty acids of the skin surface lipids, a marker of P. acnes lipase activity. A subsequent indirect anticomedogenic effect is also observed. Studies using topical clindamycin and erythromycin show that clinical improvement with these agents is accompanied by a reduction in P. acnes and free fatty acids in surface sebum (Lever L., Marks R., Drugs, Vol. 39 (5), p. 681-692, 1990). A significant side effect of topical antibiotics is the induction of bacterial resistance and cross-resistance. Recently, there have been increasing reports of P. acnes isolates that are resistant to one or more anti-acne antibiotic (most commonly erythromycin). This emergence of resistant strains can be associated with therapeutic failure (Eady, E A., Dermatology, Vol. 196, p. 59-66, 1998). Tetracyclines, erythromycin, and doxycycline are the mainstay of antibiotic treatment. However, these three antibiotics are bacteriostatic and not bactericidal, thus resulting in a higher risk of recurrence. Patients should be started at adequate doses and maintained on treatment for at least six months. Also, doxycycline may cause a photosensitivity reaction.

Oral antibiotics have been used for decades for the treatment of papular, pustular and cystic acne. These antibiotics include tetracycline (e.g., 250 and 500 mg dosage forms), erythromycin (e.g., 250, 333, 400 and 500 mg dosage forms), doxycycline or minocycline (e.g., 50 and 100 mg dosage forms), clindamycin (e.g., 75, 150 and 300 mg dosage forms), ampicillin (e.g., 250 and 500 mg dosage forms), cephalosporins such as cephalexin (e.g., 500 mg dosage forms) and trimethoprim/sulfamethoxazole (e.g., double strength (DS) tablets). Along with the problem of antibiotic resistance as described above with respect to topical antibiotics, a variety of side effects have also been observed depending on the oral antibiotic, including gastrointestinal (GI) intolerance, photosensitivity, rashes, hives, urticaria and vertigo. Oral antibiotics may be combined with topical agents, including, for example, benzoyl peroxide.

Clindamycin phosphate (Dalacin T™ solution and Cleocin-T™ gel, lotion, or solution by Upjohn, Kalamazoo, Mich.) is a macrolide lincomycin antibiotic that is bacteriostatic and can penetrate sebaceous follicles and reduce P. acnes. Clindamycin phosphate has been described, for example, in U.S. Pat. No. 3,969,516. Patients typically apply a thin film of clindamycin lotion, gel, or solution (pads) twice daily to affected areas. Clindamycin inhibits bacteria protein synthesis at the ribosomal level by binding to the 50S ribosomal subunit and disrupting the process of peptide chain initiation. In vitro studies indicate that clindamycin inhibits cultures of P. acnes at a minimum inhibitory concentration (MIC) of 0.4 mg/ml. In addition, free fatty acids on the skin surface decreases following application of clindamycin. However, cross-resistance has been demonstrated between clindamycin and erythromycin (Kilkenny, M., British Journal of Dermatology, Vol. 139, p. 840-845, 1998).

Combinations of antimicrobial agents such as antibiotics in topical formulations have been used in the treatment of acne. One of the first combination topical therapies is a mixture of 5% benzoyl peroxide with 3% erythromycin. Synergistic effects against P. acnes were observed in vitro such that the benzoyl peroxide prevented the overgrowth of staphylococci that occurs when erythromycin is used alone. Another combination topical therapy is BenzaClin™ which is a mixture of 5% benzoyl peroxide with 1% clindamycin phosphate. BenzaClin™ is used in the treatment of moderate to moderately severe facial acne. Another antibiotic combination that is commonly used is Benzamycin™. Benzamycin™ is a topical gel combination of erythromycin (3%) and benzoyl peroxide (5%). Adverse reactions associated with topical formulations of antibiotic combinations include skin irritation and dry skin.

In addition to prescription based anti-acne agents, there are also numerous over-the-counter (OTC) anti-acne agents available. Almost all acne sufferers try OTC products at least once during the course of their treatment; it is estimated that approximately 23 million people use topical OTC acne treatment options in the United States per year. In fact, most people try topical OTC treatments long before seeking the assistance of a dermatologist. Common OTC acne products include cleansers, pads, lotions, cover-up products, masks, and facials. Active agents in OTC treatments include benzoyl peroxide, resorcinol, sulfur, and salicylic acid. Resorcinol, sulfur, and salicylic acid have been attributed in helping break down blackheads and whiteheads. In addition, salicylic acid has been noted for its ability to help reduce the shedding of cells that line the follicles of oil glands, thereby reducing the occurrence of inflammatory acne. Representative examples of some OTC treatments comprising benzoyl peroxide include Oxy® 5 (5% benzoyl peroxide lotion), Benzoyl® 10 (10% benzoyl peroxide lotion), Benzashave® (5% or 10% benzoyl peroxide cream), Advanced Formula Oxy Sensitive® or Benzac® AC 2.5 or Desquam-E® or PanOxyl AQ® 2.5 (2.5% benzoyl peroxide gel), Benzac® 5 or Benzac AC® 5 or 5-Benzagel® or Desquam-E 5® or Desquam-X 5® or PanOxyl AQ® 5 or PanOxyl® 5 (5% benzoyl peroxide gel), Benzac® 10 or Benzac AQ® 10 or 10-Benzagel® or Desquam-E® 10 or Desquam-X® 10 or PanOxyl AQ® 10 or PanOxyl® 10 (10% benzoyl peroxide gel). Examples of some OTC treatments comprising salicylic acid include, for instance, Stri-Dex® pads, Fostex® cleansing pads, and Clearasil Maximum Strength® cleansing pads (2% salicylic acid pads). Moreover, many OTC treatments can produce surface exfoliation without being keratolytic, and may cause epidermal peeling without affecting the underlying acne pathological process. In addition, vitamins and minerals, including zinc, vitamin C and vitamin E, are used in the treatment of acne.

In an embodiment of the invention, XMP.629 (or a pharmaceutically acceptable salt or derivative of XMP.629) may be co-administered or concurrently administered with other anti-acne agents (one or more) in the treatment of acne. Concurrent administration or co-administration includes administration of the agents, in conjunction or combination, together, before, or after each other. XMP.629 (or a pharmaceutically acceptable salt or derivative of XMP.629) and other anti-acne agents (one or more) may be administered by the same or different routes. For example, XMP.629 (or a pharmaceutically acceptable salt or derivative of XMP.629) may be administered topically while other anti-acne agents (one or more) are administered orally or subcutaneously. Alternatively, XMP.629 (or a pharmaceutically acceptable salt or derivative of XMP.629) and other anti-acne agents (one or more) may be both administered topically. XMP.629 (or a pharmaceutically acceptable salt or derivative of XMP.629) and other anti-acne agents (one or more) may be applied to a patient's dermis sequentially, after an intermediate application, or may be given in different topical formulations e.g., gels, solutions, creams, lotions, or pads. XMP.629 (or a pharmaceutically acceptable salt or derivative of XMP.629) and other anti-acne agents (one or more) may be administered simultaneously or sequentially, as long as they are given in a manner sufficient to allow both agents to achieve effective concentrations at the site of acne. During sequential administration of XMP.629 (or a pharmaceutically acceptable salt or derivative of XMP.629) and other anti-acne agents (one or more), it is also contemplated that a time period varying from minutes to hours may intervene between the administration of the agents.

Anti-acne agents that may be co-administered or concurrently administered with XMP.629 (or a pharmaceutically acceptable salt or derivative of XMP.629) include one or more prescription based and/or over-the-counter (OTC) anti-acne treatments. Anti-acne agents used according to the invention may be in any pharmaceutically or cosmetically acceptable formulation. Preferable formulations for anti-acne agents are topical formulations, such as a gel, solution, lotion, cleanser, cream, or pad, or an oral formulation. Preferred prescription based anti-acne agents that may be used in conjunction with XMP.629 (or a pharmaceutically acceptable salt or derivative of XMP.629) include benzoyl peroxide, retinoids or retinoid derivatives, antimicrobial agents including azelaic acid and antibiotics or antibiotic combinations such as clindamycin, tetracycline, doxycycline, or erythromycin with or without benzoyl peroxide. Preferred OTC anti-acne agents that may be used in conjunction with XMP.629 (or a pharmaceutically acceptable salt or derivative of XMP.629) include benzoyl peroxide, resorcinol, sulfur, and salicylic acid.

Concurrent administration of XMP.629 (or a pharmaceutically acceptable salt or derivative of XMP.629) and other anti-acne agents (one or more) can provide more effective or enhanced treatments for acne. For example, concurrent administration of two agents may provide greater therapeutic effects than either agent provides when administered singly. As one example, concurrent administration may permit a reduction in the dosage of one or both agents with achievement of a similar therapeutic effect. Alternatively, the concurrent administration may produce a more rapid or complete anti-acne therapeutic effect than could be achieved with either agent alone. Additionally, in a case where the acne-associated bacteria has become resistant, XMP.629 (or a pharmaceutically acceptable salt or derivative of XMP.629) administration may reverse the bacterial resistance to the anti-acne agents.

A further advantage of concurrent administration of XMP.629 (or a pharmaceutically acceptable salt or derivative of XMP.629) and other anti-acne agent(s) (one or more) is the ability to reduce the amount of the agent which may have undesirable side effects, such as dermal irritation, itching, scaling, or sun sensitivity, effective for treatment. XMP.629 (or a pharmaceutically acceptable salt or derivative of XMP.629) may also improve the therapeutic effectiveness of other anti-acne agents in a variety of ways such as increased times to commence therapeutic benefit, reducing the development of resistance, and decreased durations of treatments, thus allowing wider use of the anti-acne agent.

When XMP.629 (or a pharmaceutically acceptable salt or derivative of XMP.629) is concurrently administered with other anti-acne agents, the XMP.629 (or a pharmaceutically acceptable salt or derivative of XMP.629) and the anti-acne agents may each be administered in amounts that would be sufficient for monotherapeutic effectiveness, or they may be administered in less than monotherapeutic amounts. Those skilled in the art can readily optimize effective dosages and monotherapeutic or concurrent administration regimens for XMP.629 (or a pharmaceutically acceptable salt or derivative of XMP.629) and other anti-acne agents, as determined by good medical practice and the clinical condition of the individual patient. When used to describe administration of XMP.629 (or a pharmaceutically acceptable salt or derivative of XMP.629) in conjunction with another anti-acne agent that is an antimicrobial agent such as an antibiotic, an amount sufficient for combinative therapeutic effectiveness with respect to XMP.629 (or a pharmaceutically acceptable salt or derivative of XMP.629) includes at least an amount effective to increase the susceptibility of the organism to the antimicrobial (e.g., antibiotic) anti-acne agent, and an amount sufficient for combinative therapeutic effectiveness with respect to an antimicrobial (e.g., antibiotic) anti-acne agent includes at least an amount of the antimicrobial (e.g., antibiotic) anti-acne agent that produces bactericidal or growth inhibitory effects when administered in conjunction with that amount of XMP.629 (or a pharmaceutically acceptable salt or derivative of XMP.629). Antimicrobial agents may include antibacterial, antifungal and/or antiprotozoan agents.

XMP.629 (or a pharmaceutically acceptable salt or derivative of XMP.629) may be used alone or in conjunction or combination with another anti-acne agent to treat acne, including mild, moderate or severe acne, and preferably mild or moderate acne. The Consensus Conference on Acne Classification (1990) proposed that acne grading be accomplished by the use of a pattern-diagnosis system, which includes a total evaluation of lesions and their complications (e.g., drainage, hemorrhage and pain). It takes into account the total impact of the disease. Based on a lesion count approximation, a severity grade of acne may be assigned as mild, moderate or severe. Mild acne involves comedones and/or few to several papules/pustules (e.g., +/++), and no nodules. Moderate acne involves several to many papules/pustules (e.g., ++/+++) and few to several nodules (e.g., +/++). Severe acne involves numerous and/or extensive papules/pustules (e.g., +++/++++) and many nodules (e.g., +++).

The following examples are provided for illustrative purposes and are not to be construed to limit the scope of the claims in any manner whatsoever.

### EXAMPLE 1

### Preparation and Characterization Studies

This example addresses the preparation and characterization of XMP.629. XMP.629 as well as salts and derivatives of XMP.629 can be prepared by a variety of synthetic procedures, including as described and referenced in U.S. Pat. No. 6,515,104. As described herein, XMP.629 was synthesized using a modified solid-phase procedure first described by Merrifield (Merrifield, R. B., Science, Vol. 150, p. 178-185, 1965). The α-amino group of each D-amino acid was protected with a t-butyloxycarbonyl (Boc) group. The sidechain functional groups were protected as follows: lysine was protected as a 2-chlorobenzyloxycarbonyl derivative, arginine was protected as a tosyl derivative, and glutamine was protected as a xanthyl derivative.

The peptide chain was assembled by first coupling the C-terminal amino acid, Boc-D-1-Naph (Boc-D-1 NaI—OH), to a 4-methylbenzhydrylamine (MBHA) resin support (Matsueda and Stewart, Peptides 2, p. 45-50, 1981). The MBHA resin was used for the synthesis of sequences containing C-terminal amide groups. The peptide chain was assembled by first removing the Boc-group on the C-terminal residue with trifluoroacetic acid (TFA), neutralizing with triethylamine (TEA), and coupling the next amino acid derivative in the sequence (Boc-D-Lys CI-CBZ) to the Boc-D-1-Naph-resin (Matsueda and Stewart, Peptides 2, p. 45-50, 1981), using diisopropylcarbodiimide (DIC) in the presence of 1 hydroxybenzotriazole (HOBt). Progress of the coupling reaction was monitored using a ninhydrin test (Kaiser et al., 1970), where complete coupling was indicated by a negative result. If the ninhydrin test was found to be positive, which indicates the presence of unreacted amine, the coupling was repeated, using half the amount of the amino acid derivative and half the amount of diisopropylcarbodiimide, or the resin acetylated, using acetic anhydride in the presence of diisopropylethylamine (DIPEA), before continuing on with the synthesis. When the ninhydrin test was negative, the Boc-group was removed with trifluoroacetic acid (TFA), the resin was neutralized with TEA, and the next protected amino acid derivative in the sequence (Boc-D-Ala) was coupled using the same procedure. Repetition of the coupling cycle with the remaining amino acid derivatives in the desired sequence resulted in a fully-protected peptide-resin. After completion of the synthesis, the N-terminal Boc-group was removed by treatment with TFA, the resin was neutralized with TEA and acetylated using acetic anhydride in the presence of DIPEA. The fully-protected peptide-resin was then washed and dried to constant weight.

Next, the peptide was cleaved from the resin by treatment of sub-lots of the resultant resin-peptide with liquid hydrogen fluoride, in the presence of a scavenger, which yielded the crude product. The product was then purified by a multi-step, preparative, reverse-phase HPLC process, during which the purity of the fractions was assessed by analytical HPLC. The final product was isolated as the acetate salt by lyophilization and packaged in pre-cleaned and depyrogenated Type III amber glass bottles filled with Teflon®—lined polypropylene caps. Reprocessing of any lot may be performed by repeating all or part of the final stages of the above-described process, e.g., purification or lyophilization. The XMP.629 product was also prepared by a commercial manufacturer, such as Polypeptide Laboratories, Inc., (Torrance, Calif.) under GMP manufacturing conditions.

An exemplary synthetic scheme for XMP.629 using such a solid-phase Boc strategy is summarized as follows.

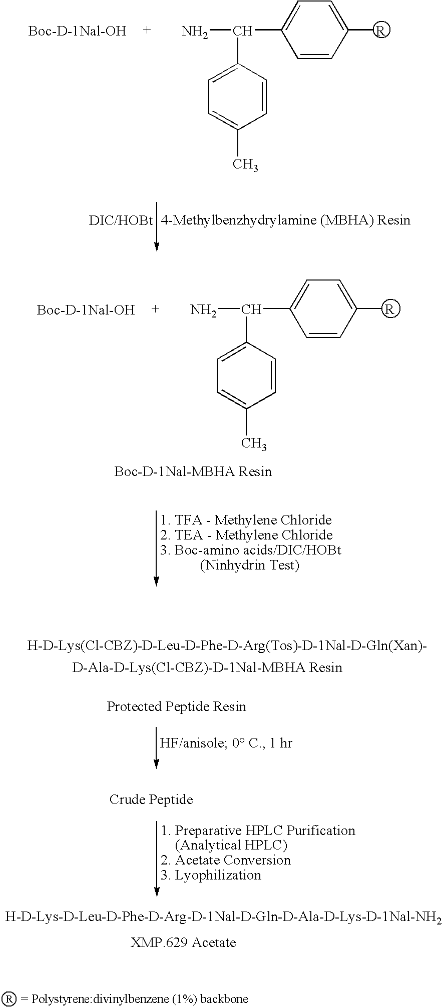

The molecular identity of the purified product was confirmed by testing. A variety of tests may be conducted and exemplary product specifications are summarized below in Table 1. Purified XMP.629 was tested in mass spectral analysis, amino acid analysis, and analytical reverse-phase HPLC with a sequenced reference standard. The purity was assessed by analytical reverse-phase HPLC techniques. Quantitative amino acid analysis was used as an assay to determine the content of the active substance in the purified product. An analytical HPLC method was used to quantify the acetate counterion in the product (as acetic acid), while a calorimetric Karl Fischer titration method was used to determine residual water in the product. The purified product was also tested for residual organic solvents by gas chromatography, residual trifluoroacetic acid by HPLC, and residual inorganic fluoride by a potentiometric method using an ion-selective electrode. Additional testing included measurement of specific optical rotation, bioburden, and endotoxin levels.

TABLE 1

| CONDUCTED TEST | | SPECIFICATION |
|---|---|---|
| 1. | APPEARANCE: | White to off-white |
| | | powder of low density |
| 2. | SOLUBILITY: | Soluble in 1% acetic acid |
| | | at a concentration of 1 mg/mL |
| | | to give a clear, colorless solution |
| 3. | IDENTITY: | Monoisotopic Mass |
| | (By Mass Spectral Analysis) | (1282.7 ± 1 m.u.) |
| 4. | IDENTITY: | Correct amino acid ratios |
| | (By Amino Acid Analysis) | with total recovery within |
| | | 10% and individual recoveries |
| | | within 15% of theory, except |
| | | for Cys, Ser, Trp, Ile, |
| | | Val and Pro, whose |
| | | recovery may be low |
| 5. | IDENTITY: | Co-elutes with Reference |
| | (By Analytical HPLC) | Standard (if available) |
| 6. | PEPTIDE PURITY: | ≧97% by area integration |
| | (By Analytical HPLC) | |
| 7. | RELATED SUBSTANCES: | ≧3% Total; no |
| | (By Analytical HPLC) | single impurity >1% |
| 8. | ASSAY (By Peptide Content): | ≧70%; preferably ≧72% |
| 9. | COUNTERION CONTENT: | ≦20%; preferably ≦18% |
| 10. | WATER (Karl Fischer): | ≦10% |
| 11. | MASS BALANCE: | 95 to 105% |
| 12. | SPECIFIC ROTATION: | Not available |
| | [χ]_{D}^{˜20} (c = 0.1, 1% acetic acid) | |
| 13. | RESIDUAL ORGANIC | ≦0.25% (w/w) Total; no |
| | SOLVENTS: | single solvent residue >0.1% (w/w) |
| 14. | TRIFLUOROACETIC ACID: | ≦0.25% |
| 15. | INORGANIC FLUORIDE: | ≦0.1% |

Purified preparations of XMP.629 were used in a variety of assays and studies as described below.

The Boc synthesis for XMP.629 as described above utilizes liquid hydrogen fluoride for cleavage and simultaneous deprotection of the protected peptide-resin precursor. To the extent that this step may influence the scalability of the process, a commercial-scale process based on solid-phase synthesis using 9-fluorenyl-methyloxycarbonyl (Fmoc-) protection of the alpha-amino groups and t-butyl-based sidechain protecting groups may be useful. A synthetic scheme for XMP.629 utilizing an Fmoc-solid-phase synthesis strategy, and using a Rink amide resin, is shown as follows.

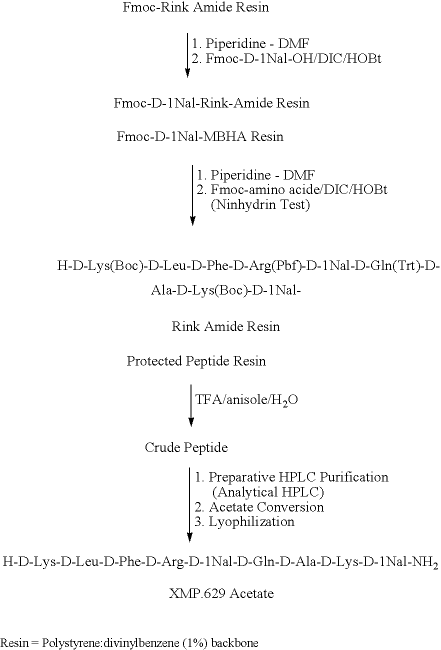

The alpha-amino group of each amino acid is protected with a 9-fluorenylmethyloxycarbonyl (Fmoc-) group, while sidechain functional groups are protected as follows: D-lysine is protected with a t-butyloxycarbonyl group; D-glutamine is protected as its trityl derivative; and D-arginine is protected with a 2,2,4,6,7-pentamethyldihydrobenzofuran-5-sulfonyl group. The peptide chain is assembled by first removing the Fmoc-group on the Rink amide resin using a solution of piperidine in dimethylformamide (DMF). After washing the resin with DMF and isopropanol, the first amino acid derivative in the sequence [Fmoc-D-(1-naphthyl)-alanine; approximately 2 equivalents] is coupled to the resin for a minimum of 1 hour using diisopropylcarbodiimide (DIC; approximately 2 equivalents) in the presence of 1-hydroxybenzotriazole (HOBt; approximately 2 equivalents) in DMF as solvent. After washing the resin, completion of the coupling reaction is verified using the ninhydrin test [E. Kaiser, et al., Analytical Biochemistry, Vol. 34, p. 595, (1970)], which should be negative, indicating complete coupling. If the ninhydrin test is found to be positive, indicating the presence of unreacted amine, the coupling is repeated, using approximately half the amount of the amino acid derivative in the presence of HBTU, HOBt and diisopropylethylamine (DIPEA) in DMF as solvent; or the resin is acetylated, using acetic anhydride in the presence of pyridine in DMF as solvent, before continuing with the synthesis.

When the ninhydrin test is negative, the Fmoc-group is removed with piperidine, and the next protected amino acid derivative in the sequence [N-α-Fmoc-N-ε-(t-butyloxycarbonyl)-D-lysine] is coupled using the procedure described above. Repetition of the coupling cycle with the remaining amino acid derivatives in the desired sequence results in the fully-protected peptide-resin. After completion of the synthesis, the N-terminal Fmoc-group is removed by treatment with piperidine. The protected peptide-resin intermediate is then washed and dried to constant weight. All of the amino acids in the sequence are of the D-configuration.

In the second stage of the process, the peptide is cleaved from the resin with trifluoracetic acid in the presence of anisole and water (97:0.5:2.5, v/v) followed by precipitation with ether. To give the crude product, which is washed with ether and dried to constant weight under vacuum.

In the final stage of the process, the crude product is purified by the same, two-step, preparative, reverse-phase HPLC process used in the Boc process described above. The final purified product solution may be lyophilized to yield XMP.629 Acetate.

### EXAMPLE 2

### Antimicrobial Activity Studies

This example addresses studies relating to the antimicrobial activities of XMP.629. An in vitro assessment of antibiotic activity in XMP.629 and various known antibiotics against a representative panel of bacterial strains associated with acne was conducted. A broth dilution methodology was employed using guidelines established by National Committee for Clinical Laboratory Standards (NCCLS) for anaerobic organisms to establish an antibacterial profile for XMP.629. Assays to determine the minimal bactericidal concentrations (MBC) and minimal inhibitory concentration (MIC) were conducted for XMP.629 and other known antibiotics. Furthermore, test to determine post antibiotic effect (PAE) of XMP.629 were done.

Inoculum from a representative panel of bacterial strains associated with acne, such as P. acnes, P. avidum, P. granulosum, and Staphylococcus epidermis was prepared by suspending approximately 5-10 isolated bacterial colonies in pre-reduced MicroScan® Inoculum Water (Dade Behring, Deerfield, Ill.), and growing the bacterial suspension to log phase (approximately 48 hrs). An inoculum of between 1-5×10⁶/mL was achieved by adding 100 μL of the suspension to 25 mL Brain Heart Infusion Broth (BHI) (Anaerobe Systems, Morgan Hill, Calif.).

A stock solution of XMP.629 dissolved in water at a concentration of 4.0 mg/mL was prepared. Dilute solutions of XMP.629 at concentrations ranging from about 1.25 to about 160 μg/mL were made in either water or formulation buffer (130 mM sodium chloride, 0.2% P103 (poloxamer 333), and 0.10% EDTA, pH 7.3). Aqueous stock solutions of clindamycin and erythromycin were prepared in water.

For the MBC assay, 100 μL of XMP.629 or antibiotic in either water or formulation buffer was dispensed into 12×75 mm polypropylene tubes followed by 900 μL of inoculated BHI. Each tube, containing a final volume of 1 mL, was mixed by vortexing. Control tubes containing only water or formulation buffer were prepared for each isolate. Initial growth controls were determined by removing 10 μL from the water control tube and diluting it by 100 fold. From this dilution, 10 μL was used for inoculation and for spreading over the surface onto Laked Blood Agar (LBA) (Anaerobe Systems, Morgan Hill, Calif.) plates. The tubes and plates were incubated in an anaerobic atmosphere at 37° C. for 48 h. After incubation, the tubes were mixed and 10 μL samples were removed from each tube for additional plating. These plates were then further incubated in an anaerobic atmosphere at 37° C. for 48-72 h and the resulting colonies were counted. After adjusting for the dilution factor, the lowest concentration of drug or combination of drugs reducing the inoculum by ≧99.9% was considered to be the minimum bactericidal concentration (MBC). MBC₅₀ and ₉₀ values represent where 50 and 90%, respectively, of the tested strain's MBC values are equal to or less than the indicated concentration. The lowest concentration of antibiotic(s) that inhibited visible growth in the culture tubes was considered to be the minimal inhibitory concentration (MIC).

Results from two independently conducted studies, summarized in Table 2, demonstrated the killing activity of XMP.629 against a panel of representative Propionibacterium isolates. XMP.629 exhibits significant antibacterial activity against some of the agents associated with acne, such as Propionibacterium. For example, XMP.629 possesses antibacterial activity against P. acnes, P.avidum, P. granulosum, and Staphylococcus epidermis. XMP.629, dissolved in either water or formulation buffer and, exhibited activity against multiple strains of representative Propionibacterium, such as P. acnes, P. avidum, and P. granulosum. XMP.629 was also found to possess rapid antimicrobial activity against gram negative and gram positive organisms, and against fungi.

TABLE 2

| | | Formulation Buffer Solvent | | | | Water Solvent | | | |
|---|---|---|---|---|---|---|---|---|---|
| Isolate | | MIC | | MBC | | MIC | | MBC | |
| Deposit Number | Organism | Study 1 | Study 2 | Study 1 | Study 2 | Study 1 | Study 2 | Study 1 | Study 2 |
| ATCC 11827 | P. acnes | 0.5 | 1 | 1 | 1 | 2 | 2 | 2 | 2 |
| ATCC 6919 | P. acnes | 0.5 | 1 | 1 | 0.50 | 2 | 2 | 4 | 2 |
| RMA 12689 | P. acnes | 1 | 1 | 2 | 2 | 2 | 2 | 4 | 2 |
| RMA 12732 | P. acnes | 2 | 2 | 2 | 2 | 4 | 4 | 4 | 2 |
| RMA 12908 | P. acnes | 2 | 2 | 2 | 2 | 4 | 2 | 4 | 4 |
| RMA 13399 | P. acnes | 0.5 | 2 | 1 | 2 | 2 | 4 | 4 | 4 |
| RMA 9833 | P. acnes | 0.5 | 1 | 1 | 2 | 1 | 2 | 2 | 2 |
| RMA 13009 | P. avidum | 0.25 | 0.25 | 0.50 | 1 | 1 | 1 | 1 | 1 |
| RMA 8375 | P. avidum | 0.25 | 0.25 | 0.50 | 1 | 0.5 | 1 | 1 | 2 |
| RMA 8376 | P. avidum | 0.5 | 0.25 | 0.50 | 2 | 1 | 1 | 2 | 2 |
| RMA 9497 | P. avidum | 0.25 | 0.25 | 1 | 2 | 2 | 1 | 2 | 4 |
| RMA 5740 | P. granulosum | 1 | 0.25 | 2 | 0.25 | 2 | 1 | 4 | 1 |
| RMA 6867 | P. granulosum | 0.5 | 4 | 2 | 4 | 1 | 4 | 2 | 4 |
| RMA 7093 | P. granulosum | 1 | 0.25 | 2 | 0.25 | 2 | 1 | 2 | 0.5 |
| RMA 11787 | P. granulosum | 2 | 0.25 | 2 | 0.25 | 2 | 1 | 2 | 1 |
| GeoMean | | 0.7 | 0.7 | 1.2 | 1.10 | 1.7 | 1.7 | 2.41 | 1.9 |
| Values | | | | | | | | | |

Note:
MBC = Minimum Bactericidal Concentration;
MIC = Minimum Inhibitory Concentration.
Values are in units of μg/mL.

In additional tests performed in similar manner to that described above, XMP.629 showed activity against other additional organisms that are implicated in the development of acne, such as Corynebacterium, Enterococcus, S. aureus, P. aeruginosa, and Candida. Results from a comparison study of XMP.629 with other known antibiotics are illustrated below in Table 3.

TABLE 3
MIC₉₀ of Standard Topical Agents and XMP.629

| | | XMP.629 | | | | | | |
|---|---|---|---|---|---|---|---|---|
| Organisms | XMP.629 | in FB | Clindamycin | Erythromycin | Vancomycin | SSD | Mupirocin | 594AN |
| P. acnes | 1 μg/mL | N/A | 16 | >256 | 2 | N/A | NE | 2.3 |
| Corynebacterium | 0.25 | <0.25 | N/A | 0.5 | 0.5 | 50 | NE | N/A |
| Enterococcus | 4 | 0.5 | NE | NE | 1.0/NE | 50 | NE | N/A |
| S. aureus | 8 | 0.06 | 0.5 | >4 | 4 | 100 | 4 | N/A |
| P. aeruginosa | 32 | 0.5 | NE | NE | NE | 50 | NE | N/A |
| Candida | 1 | 0.5 | NE | NE | NE | 100 | NE | N/A |

Note:
XMP.629 data is reported as MBC values, which are typically higher than MIC values. The presence of formulation buffer alone had no significant activity against the organisms tested above. Results for S. epidermidis were similar to those of S. aureus. 594AN data is reported as geometric mean MIC values.
N/A = Not available;
NE = Not Effective.

Acne vulgaris may involve a microbial consortium. Although P. acnes may be a prominent member of such a consortium, other uncharacterized bacteria may also participate. The microbial activity of XMP.629 as an acetate gel as described in Example 5 and other commonly used antibiotics was evaluated against a panel of human pathogens using standardized NCCLS protocols. XMP.629 acetate gel was found to have potent activity against gram-negative organisms, with MIC values typically ranging from 2-8 μg/mL as shown in Table 4. Lesser potency was observed against the gram-negative organisms B. fragilis, B. cepacia, P. mirabilis and S. marcescens, which are often refractory to many antimicrobial compounds.

TABLE 4

| | Minimum Inhibitory Concentrations (μg/mL) | | | |
|---|---|---|---|---|
| | XMP.629 | Ceftriaxone | Ciprofloxacin | Doxycycline |
| Actinobacillus | 4.00 | NT | ≦1 | NT |
| actinomycetemcomitans 43718 | | | | |
| Bacteroides fragilis 25285 | 16.0 | 2.50 | NT | NT |
| Burkholderia cepacia 25416 | 32.0 | NT | 1.00 | NT |
| Escherichia coli 0111:B4 | 2.00 | ≦0.063 | ≦0.063 | 2.00 |
| Escherichia coli 07:K1 | 4.00 | ≦0.25 | ≦0.25 | NT |
| Klebsiella pneumoniae 19645 | 2.00 | >64 | 0.13 | 32.0 |
| Klebsiella pneumoniae 29011 | 8.00 | 0.13 | ≦0.063 | 32.0 |
| Porphyromonas gingivalis 33277 | 4.00 | NT | ≦1 | NT |
| Prevotella intermedia 25261 | 2.00 | NT | ≦1 | NT |
| Proteus mirabilis 29852 | >64 | ≦0.063 | ≦0.063 | 64.0 |
| Pseudomonas aeruginosa 19660 | 8.00 | NT | 0.13 | NT |
| Pseudomonas aeruginosa 27853 | 4.00 | 8.00 | ≦1 | 32.0 |
| Pseudomonas aeruginosa 9027 | 8.00 | NT | 0.13 | NT |
| Pseudomonas aeruginosa | 8.00 | 16.0 | ≦1 | NT |
| XS-65 (MDR) | | | | |
| Salmonella ryphimurium SL770 | 4.00 | NT | ≦1 | NT |
| Serratia marcescens 13880 | 64.0 | 0.25 | 0.06 | 8.00 |
| Serratia marcescens 14756 | 32.0 | NT | NT | NT |

XMP.629 acetate gel showed consistent potent activity against gram-positive organisms, including the vancomycin resistant Enterococcus faecium (VRE) strain, as shown in Table 5. XMP.629 acetate gel yielded an MIC of 2.0 μg/mL in comparison to an MIC of 64 μg/mL or greater with the comparator antibiotics. These results suggest that the target receptor for XMP.629 is unique and does not cross-react with other antimicrobial peptides. XMP.629 acetate gel was also highly effective against a methicillin resistant S. aureus (MRSA) strain and was equipotent against both a wild type and a multi-drug resistant strain of P. aeruginosa as shown.

These data demonstrate that the susceptibility of tested organisms to other standard antibiotics did not affect susceptibility to XMP.629 acetate gel. In addition, the fact that the two strains of Pseudomonas showed equal sensitivity to the compound indicated that XMP.629 was not a substrate for the efflux drug pumps used by these resistant strains.

TABLE 5

| | Minimum Inhibitory Concentrations (μg/mL) | | | |
|---|---|---|---|---|
| | XMP.629 | Ciprofloxacin | Vancomycin | Oxacillin |
| Clostridium difficile 9689 | 2.00 | NT | 1.00 | NT |
| Enterococcus faccium 700221 (VRE) | 2.00 | 64.0 | >64 | >64 |
| Enterococcus hirae 9790 | 2.00 | ≦1 | NT | NT |
| Macrococcus caseolyticus 29750 | 2.00 | ≦1 | NT | NT |
| Propionibacterium acnes 6919 | 1.00 | NT | 2.00 | NT |
| Staphylococcus aureus BAA-38 (MRSA) | 2.00 | 0.13 | 1.00 | 8.00 |
| Staphylococcus aureus 6258 | 2.00 | NT | 2.00 | NT |
| Staphylococcus aureus 11371 | 2.00 | 0.50 | 1.00 | NT |
| Staphylococcus aureus 12598 | 2.00 | ≦1 | 1.25 | NT |
| Staphylococcus aureus 19636 | 2.00 | ≦1 | ≦1 | 1.00 |
| Streptococcus mutans 25175 | ≦1 | 2.00 | 2.00 | NT |
| Streptococcus pneumoniae 6303 | 8.00 | NT | NT | NT |
| Streptococcus pneumoniae 10031 | 8.00 | ≦1 | NT | NT |
| Streptococcus pneumoniae 35088 | 2.00 | ≦1 | NT | NT |
| Streptococcus sobrinus 27607 | 2.00 | 2.00 | 1.00 | NT |

Kill curves were generated by incubating P. acnes strain ATCC 6919 with formulation buffer solutions of XMP.629 or other antibiotics at concentrations of 1, 2 and 4 mg/mL. Samples were removed and plated at the indicated time periods. After incubation, colonies were counted and the kill kinetics determined for each drug.

Results from the kill assay showed that the killing of P. acnes by XMP.629 was time and concentration dependent. For example, at a higher concentration of 4 μg/mL, a rapid reduction of viable cells in 8 hours to undetectable levels was observed. At 19 hours, all concentrations tested effectively reduced the cultures to no detected organisms. In additional kill curve studies against P. acnes, XMP.629 was bactericidal within 6 hours of exposure. The results of kill kinetic studies indicated that the lethal effects of XMP.629 were demonstrated with up to 10 hours of contact to kill the microorganisms, depending on dose.

To determine the influence of antibiotic resistance on the MBC values for XMP.629, a further MBC study was conducted against clindamycin sensitive and clindamycin resistant Propionibacterium strains. For comparison, additional Propionibacterium strains, were tested. MBC values for XMP.629 for these tested strains are presented in Table 6.

Results show that the MBC₅₀ for XMP.629 in formulation buffer against P. acnes was 1 μg/mL and the MBC₉₀ was 2 μg/mL. Of the 36 strains tested, 16 were clindamycin resistant (MIC>64 μg/mL). There were no apparent differences in susceptibility to XMP.629 between the clindamycin sensitive and resistant strains. Clindamycin resistance did not confer any resistance to XMP.629. The number of P. avidum and P. granulosum strains tested were insufficient to calculate meaningful MBC values, but the data are also summarized in Table 6.

TABLE 6

| | Number of | | | |
|---|---|---|---|---|
| | strains/clinical | MBC₅₀ | MBC₉₀ | |
| Species | isolates tested | (μg/mL) | (μg/mL) | Range |
| Propionibacterium acnes | 36 | 1 | 2 | 0.25-2 |
| Clindamycin sensitive | 20 | 1 | 2 | 0.25-2 |
| Clindamycin resistant | 16 | 1 | 2 | 1-2 |
| P. granulosum | 4 | 0.25 | 4 | 0.25-4 |
| P. avidum | 4 | 1 | 2 | 1-2 |

In additional studies, P. acnes strains resistant to erythromycin, methicillin or vancomycin did not affect the MBC values of these strains to XMP.629, suggesting that it has a different mechanism of action than that of the other antimicrobials tested.

The MBC values for XMP.629 against six individual Propionibacterium acnes strains were obtained as shown in Table 7. Both the MBC₅₀ and MBC₉₀ for an XMP.629 composition as described in Example 5 (see Table 12) against P. acnes was 2 μg/mL. Of these selected strains tested, again there were no apparent differences in susceptibility to XMP.629 between the clindamycin sensitive and resistant strains.

TABLE 7

| | MIC | MBC |
|---|---|---|
| Propionibacterium acnes Strain | μg/mL | μg/mL |
| ATCC 6919 | 1 | 2 |
| Clindamycin Resistant C767 | 1 | 2 |
| Clindamycin Resistant C769 | 1 | 2 |
| UD 738 | 0.5 | 2 |
| Clindamycin Resistant C768 | 2 | 4 |
| RMA 12908 | 1 | 2 |
| Clindamycin Resistant C678 | 1 | 4 |
| Clindamycin Resistant C770 | 0.5 | 2 |
| RMA 9833 | 0.5 | 1 |
| RMA 12689 | 1 | 2 |
| ATCC 11828 | 1 | 2 |
| Geo. Mean | 0.95 | 2.3 |

Post-antibiotic effect (PAE) studies with XMP.629 were conducted according to the procedure described by Craig, W. and S. Gudmundsson, “Postantibiotic Effect,” p. 296, In: Lorian, V. (ed.) Antibiotics in Laboratory Medicine, 4^{th} ed. The post antibiotic effect measures the duration that an antibiotic affects target organisms after contact with the bacteria. In this study, an initial concentration of 5×10⁶ CFU/mL P. acnes (ATCC 6919) and 1 μg/mL were incubated for 15, 30, or 60 minutes and then diluted 100-fold in BHI. The treated organisms and matched untreated controls were incubated for 7 hours. Samples were taken and counted at hourly intervals. Recovery was determined by plate counts over time.

The postantibiotic value is the difference in time between the controls and treated cultures to increase 1 log₁₀. Because of the long generation time (slow growth) of the organism, the recovery time was unusually long, >12 hours. This may have influenced the results as typical assays are completed within 5-6 hours. The results indicated that the controls, with and without formulation buffer, all increased by one log by approximately 7 hours. Similar results were seen for the bacteria treated for only 15 minutes with XMP.629. However, cultures in contact with XMP.629 for 30 minutes were somewhat reduced in numbers and for 60 minutes more reduced in numbers from the matched controls at the time of antibiotic removal and did not cover over the time course of the study (12 hours). Since the treated groups did not increase by the required log₁₀, an accurate PAE value could not be assigned (≧12 hours). However, the study did demonstrate that XMP.629 does not have a continuing inhibitory, but not lethal, effect on susceptible bacteria for more than 5 hours after contact with peptide when the residence time was greater than 15 minutes. Additional postantibiotic effect studies of XMP.629 demonstrated that after exposure of P. acnes to 0.5 to 2.0 MBCs for 15 minutes, the affected cells did not recover, even after 24 hours of incubation.

Studies were undertaken to determine whether XMP.629 enhanced the effectiveness of commonly known antibiotics used against P. acnes. A checkerboard assay was performed with increasing concentrations of erythromycin and clindamycin with XMP.629 according to the following procedure.

Polypropylene tubes (12×75 mm) were arranged in a checkerboard-like fashion and 100 μL of one of the actives was dispensed into each tube, followed by XMP.629 and the inoculated medium. Each tube, contained a final volume of 1 mL, was mixed by vortexing. A 10 μL sample was removed from a control tube and diluted 100 fold in water and 10 μL aliquots were spread onto a BRU plate. The tubes and plates were incubated at 37° C. for 48 h. After incubation, the tubes were mixed and 10 μL samples were again removed from each tube for plating. All plates were incubated at 37° C. for approximately 48-72 h and the resulting colonies were counted. After adjusting for the dilution factor, the lowest concentration of drug or combination of drugs reducing the inoculum by ≧99.9% was considered to be the minimum bactericidal concentration (MBC). The lowest concentration of antibiotic(s) that inhibited visible growth in the culture tubes was considered to be the minimal inhibitory concentration (MIC). The relationship between the drug combinations is calculated as the fractional inhibitory concentration (FIC) or bactericidal concentration (FBC) (Eliopoulos, G. and R. Moellering, “Antimicrobial Combinations,” p. 338, In: Lorian, V. (ed.) Antibiotics in Laboratory Medicine, 4^{th} ed.).

The antibiotic combinations were challenged with a clindamycin resistant strain UPC686 (MIC>64 μg/mL), strain ATCC 6919 that had been converted to an intermediate-erythromycin strain (MIC=64 μg/mL) and a clindamycin/erythromycin sensitive strain, UP2022 (MIC<1 μg/mL). For each strain, the MBC for XMP.629=1 μg/mL. The addition of the other antibiotics, up to 64 μg/mL, had no effect on XMP.629's bactericidal potential (FBC=2). For strains UPC686 and UP2022, MIC values for erythromycin/clindamycin were unaffected (FIC=2). The results of these relationships are described as indifference, where neither compound influences, either positively or negatively, the activity of the other. However, for the erythromycin intermediate-resistant strain, the FIC was 0.75 (FBC=2) for the XMP.629/erythromycin combination, considered an additive affect on growth inhibition of P. acnes but demonstrating no enhanced bactericidal properties. In additional experiments, a variety of antibiotics were tested in combination with XMP.629 and the results are shown in Table 8 and Table 9.

TABLE 8

| | | MBC, μg/mL | | |
|---|---|---|---|---|
| | | | Penicillin+ | |
| Strains | Penicillin | XMP.629 | XMP.629 | FIC |
| Staphylococcus | | | | |
| aureus | | | | |
| 076U-550 | <0.06 | 8 | <0.06 | ND |
| 15031-1857 | >10 | 4 | >10/4 | 2 - Indiffer- |
| | | | | ent |
| 230U-579 | >10 | 8 | 1.25/4 | 0.56 - Additive |
| 342U-116 | >10 | 8 | 5/4 | 0.75 - Additive |
| 314U-148 | >10 | 8 | 0.06/4 | 0.50 - Additive |
| E. coli | | | | |
| 4104 LF | >10 | 8 | 5/4 | 0.75 - Additive |
| 706U2-1081 | >10 | 16 | 2.5/8 | 0.62 - Additive |

TABLE 9

| | | | Antibiotic + | | |
|---|---|---|---|---|---|
| Antibiotic | MBC, μg/mL | XMP.629 MBC | XMP.629 | FIC | Result |
| | | E. coli 4104 LF | | | |
| Ampicillin | 32 | 16 | 4/≦2 | ≦0.25 | Synergy |
| Cefuroxime | 8 | 16 | ≦4/≦2 | ≦0.625 | ND |
| Ceftriaxone | ≦4 | 16 | ≦4/≦2 | ≦0.625 | ND |
| Neomycin | 1 | 16 | ≦0.25/≦2 | ≦0.375 | Synergy |
| Ofloxacin | ≦0.125 | 16 | ≦0.125/≦2 | ND | ND |
| | | Staphylococcus aureus 342U-116 | | | |
| Ampicillin | 8 | 8 | 4/4 | 1 | Additive |
| Cephalothin | 1 | 8 | 0.5/2 | 0.75 | Additive |
| Ceftriaxone | >4 | 8 | 0.5/4 | 0.5625 | ND |
| Neomycin | 1 | 8 | 0.125/1 | 0.25 | Synergy |
| Ofloxacin | 1 | 8 | 0.25/2 | 0.375 | Synergy |
| Oxicillin | 1 | 8 | 0.25/2 | 0.375 | Synergy |

### EXAMPLE 3

### Resistance Studies

This example addresses studies relating to resistance. The development of resistance is a known potential hazard with existing antibiotic therapies for the treatment of acne. Experiments were conducted to determine whether resistance was developed by P. acnes exposed to XMP.629.

To develop strains of P. acnes resistant to erythromycin, clindamycin and XMP.629, a P. acnes strain (ATCC 6919) was continuously passed in increasingly higher concentrations of each antibiotic. Control, naive, susceptible cultures were challenged in parallel with the treated organisms. Initially, approximately 5×10⁶ cfu/mL of P. acnes in Brain Heart Infusion Broth (BHI) (Anaerobe Systems, Morgan Hill, Calif.). was treated with increasing concentrations of the drugs. After incubation for 48 h, the contents were plated on Brucella agar (BRU) (Anaerobe Systems, Morgan Hill, Calif.) and incubated for 72 h. Colonies from the highest drug concentration were resuspended in BHI and again challenged with drug. Resistance to the antibiotic is determined by visible growth in the culture tubes, i.e. increase in the minimal inhibitory concentration (MIC), at least 4-fold higher than the control.

Repeated sub-cultures of P. acnes ATCC 6919 in sub-lethal/inhibitory concentrations of XMP.629, erythromycin and clindamycin were routinely performed until sustainable cultures capable of growth in 64 μg/mL of erythromycin and clindomycin or >4 μg/mL XMP.629 were obtained. For erythromycin, intermediate-resistant (IR) strains (MIC=64 μg/mL) emerged after 11 passages and resistant (R) strains (MIC>64 μg/mL) emerged after 14 passages. MIC values for matched, untreated, control organisms remained at 4 μg/mL. In the presence of sub-lethal concentrations of XMP.629, culture experiments were halted after 17 passages when no resistant colonies were formed; this represents a resistance rate of <1.2×10⁸. The results are summarized below in Table 10 and indicate that development of resistance to XMP.629 appears to be a rare event, requiring target or drug modifications that are not readily inducible by the bacteria.

TABLE 10

| Compound | MIC/MBC | Generation | Resistance Rate |
|---|---|---|---|
| Erythromycin IR | 64 μg/mL | 11 | 5 × 10⁻⁸ |
| Erythromycin R | >64 μg/mL | 14 | 7 × 10⁻⁸ |
| Clindamycin | >64 μg/mL | ND@17 | <1.1 × 10⁻⁸ |
| XMP.629 | >4 μg/mL | ND@17 | <1.1 × 10⁻⁸ |

Note:
ND = Not Determined

In additional resistance studies, culture experiments were performed in an attempt to generate XMP.629 resistant strains of P. acnes by continued passage of ATCC 6919 in increasingly higher concentrations of antibiotic. Using 1, 2, and 4 mg/mL of a XMP.629 composition as described in Example 5 (see Table Table 12), naive and susceptible cultures then were challenged in parallel with the treated organisms to determine MIC and MBC after every fifth treatment. As shown in Table 11, after twenty passages there appeared to be no significant difference between XMP.629 affected organisms and naive control strains.

TABLE 11

| | MIC | MBC |
|---|---|---|
| P. acnes strain | μg/mL | μg/mL |
| Mutant 1 | 1 | 2 |
| ATCC 6919 control | 1 | 2 |
| Mutant 5 | 1 | 2 |
| ATCC 6919 control | 1 | 2 |
| Mutant 10 | 2 | 4 |
| ATCC 6919 control | 2 | 4 |
| Mutant 15 | 1 | 4 |
| ATCC 6919 control | 1 | 4 |
| Mutant 20 | 1 | 2 |
| ATCC 6919 control | 2 | 4 |

### EXAMPLE 4

### Additional Activity Studies

This example addresses studies relating to additional activities of XMP.629, including its known endotoxin-related activities. For example, XMP.629 exhibited endotoxin neutralizing activity in a murine model of endotoxemia. For these studies, groups of 15 male CD-1 mice were challenged with 25 mg/kg E. coli 0111 :B4 lipopolysaccharide via tail vein injection. Immediately following endotoxin challenge, mice were treated intravenously with XMP.629 at 0.07, 0.2, 0.65 mg/kg or saline. Animals were observed twice daily and mortality was recorded for seven days. XMP.629 showed a dose-dependent effect on survival in endotoxin-challenged mice and complete protection was observed at 0.65 mg/kg.

In additional studies in an acute peritonitis model, mice were challenged intraperitoneally with 1.4×10⁷ CFU E. coli 07:K1 and treated intraperitoneally with XMP.629 at 1, 3, or 10 mg/kg or saline. Mice were observed twice daily and mortality was recorded for seven days. Statistical comparisons between the treatment groups and saline were performed using a Chi Square test. XMP.629 showed a significant dose-dependent effect on survival in mice challenged with E. coli 07:K1. At 10 mg/kg, 87% protection was observed. In additional studies, XMP.629 was safely administered at a dose of 20 mg/kg intravenously.

In this peritonitis study, XMP.629 showed a dose dependent effect on survival.

### EXAMPLE 5

### Compositions of XMP.629

Acne treatment compositions presented herein comprising XMP.629 (or a pharmaceutically acceptable salt or derivative of XMP.629) may be in any cosmetic, physiologically or pharmaceutically acceptable formulation. In a preferred embodiment, the present compositions are formulated for topical applications. In a more preferred embodiment, the present compositions are formulated as a gel.

Exemplary gel compositions are described in Table 12 below (as well as in the following Examples). A variety of representative compositions are prepared at a variety of XMP.629 concentrations, including 0.01%, 0.05%, and 0.10%, for subsequent in vitro, animal and human testing and use. The concentration of various components, along with the active ingredient (e.g., XMP.629) including buffer components, salts, tonicity agents, solvents, gelling agents, preservatives, chelating agents, wetting agents and/or surfactants, including, for example, a variety of buffers (e.g., acetate buffer) and/or salts, and/or tonicity agents (e.g., sodium chloride), propylene glycol (e.g., 0-10% weight to weight), hydroxyethylcellulose (e.g., 0-1.5% or 0.4-1.5% weight to weight), benzalkonium chloride (e.g., 0-0.01% weight to weight), EDTA (e.g., 0-0.5% weight to weight), or various poloxamer surfactants (e.g., 0-1% weight to weight) are varied and/or optimized, including by testing in stability studies and/or activity studies.

TABLE 12

| Component | % (w/w) |
|---|---|
| XMP.629 Acetate | 0.01-0.10 |
| EDTA disodium, dehydrate | 0.15 |
| Propylene Glycol | 2.0 |
| Sodium Chloride | 0.78 |
| Benzalkonium Chloride | 0.005 |
| Poloxamer 333 (Pluronic P-103) | 0.20 |
| Hydroxyethylcellulose, 250 HHX | 1.25 |
| Sodium Acetate buffer (pH 6.0)¹ | q.s. ad 100 |

¹Buffer component may be 0.005% w/w of acetic acid (36% w/w), 0.13% w/w sodium acetate, trihydrate and purified water q.s. ad 100.

XMP.629 showed stability and biological activity in a variety of formulations. For initial studies, XMP.629 was soluble in aqueous solution at a variety of concentrations, including 5 and 20 mg/mL, and remained stable as indicated by biological activity in aqueous solution for greater than 6 months at 4° C. In addition, XMP.629 was substantially resistant to protease degradation. In additional studies, a variety of formulations of XMP.629 were tested for stability under various temperatures, such as 25° C., 40° C., and 50° C., and at various time points, such as 0.5, 1, and 2 months. Observations recorded under such varying time points and temperatures, such as appearance, pH, viscosity, and concentration, indicated the overall stability of these formulations.

An exemplary manufacturing procedure for an XMP.629 acetate gel is as shown in Table 12 is described as follows. In a manufacturing vessel, a 36% acetic acid solution, sodium acetate trihydrate, and purified water are added. The solution is mixed with a propeller until a clear solution is obtained. The pH of the solution is checked and confirmed to be within a pH range of 6.0±0.2. With continuous propeller mixing, edetate disodium dihydrate (EDTA), sodium chloride, benzalkonium chloride (BAK), propylene glycol (PG), and poloxamer 333 (Pluronic P-103) are added and mixed until the solution is clear. With continuous propeller mixing, XMP.629 acetate is added and mixed until the solution is clear. With continuous propeller mixing, hydroxyethylcellulose 250HHX (HEC) is dispersed and mixed until a smooth and homogenous clear gel is formed.

A variety of articles, kits and containers, including container-closure-systems, may be utilized for compositions of XMP.629, including where the compositions are gels, lotions, creams, solutions (e.g., washes) and/or where the compositions are presented in a wipe, swab, aerosol, spray, gel stick, patch or impregnated dressing. Exemplary container-closure systems for four packaging various presentations are described for XMP.629 acetate gel (e.g., from about 1 g to about 100 g) which is filled and packaged in commercial package sizes such as 20, 30 and 45 g tubes, and a physician's sample size such as a 3.5 g tube as follows: (a) 3.5 g tube—Laminate tube (Glaminate®) with sealed orifice Glamaseal®, white polypropylene cap, nominal 3-5 gram fill; (b) 20 g tube—Laminate tube (Glaminate®) sealed orifice Glamaseal®, white polypropylene cap, nominal 15 gram fill; (c) 30 g tube—Laminate tube (Glaminate®) sealed orifice Glamaseal®, white polypropylene cap, nominal 20 gram fill; (d) 45 g tube—Laminate tube (Glaminate®) sealed orifice Glamaseal®, white polypropylene cap, nominal 45 gram fill.

Sample size of the composition may be varied as desired and a number of container sizes can be utilized, including for individual samples as well as daily, weekly or monthly (e.g., 1 month, 2 month or 3 month) samples and corresponding containers.

### EXAMPLE 6

### Additional Compositions and Testing

This example addresses various compositions, including topical formulations of XMP.629, and including skin penetration properties of XMP.629 as formulated. The composition of topically applied formulations may play an important role in drug bioavailability and this may be assessed as described in this example.

A variety of compositions (e.g., formulations) were prepared, including liquid formulations, gel formulations, including those containing a small amount of ethanol and various gelling agents, lotion formulations, including those containing different types of emulsifiers, and additional formulations, including those containing different types of penetration enhancers.

The solubility of XMP.629 acetate in various solvents was also evaluated. Formulation development included preparation of vehicle formulations, including for short-term stability studies. Stable vehicle formulations were then added to the drug substance.

A variety of formulations and their respective vehicles were evaluated in anti-microbial activity or effectiveness tests, or in skin penetration tests.

Data from skin penetration tests, antimicrobial activity tests and/or physical evaluation tests (e.g., pH, viscosity and skin feel) were used to evaluate various formulations.

Additional formulations, including those containing different amounts of PG were further evaluated. A preferred formulation, 1121-77E, contains 2% PG.

Solubility studies were conducted using a variety of solvent systems and/or skin penetration enhancers. Solubility at room temperature (mg/ml) of XMP.629 acetate, for example in sodium acetate buffer (pH 6.0) was >1.64, for purified water was <1.66, for propylene glycol was 10.56<S<12.5, for transcutol P was <0.17, and for ethanol, 200 proof was <2.20.

For example, a variety of gel, lotion and cream compositions (e.g., formulations) were prepared with and without XMP.629, including for example, the following formulations containing the listed ingredients given in weight percentages [w/w (g)] based on the total weight of the composition: 1121-8A (gel) containing: (A) 56.9% sodium acetate buffer, 0.10% EDTA disodium, and (B) 10.0% propylene glycol, 1.0% polysorbate 20, 30% ethanol alcohol (200 proof, and (C) 2% HPC; 1121-8B (gel) containing: (A) 46.9% sodium acetate buffer, 0.10% EDTA disodium, and (B) 20.0% propylene glycol, 1.0% polysorbate 20, 30.0% ethanol alcohol (200 proof, and (C) 2.0% HPC; 1121-8C (gel) containing: (A) 46.9% sodium acetate buffer, 0.10% EDTA disodium, and (B) 20.0% propylene glycol, 1.0% polysorbate 20, 20.0% ethanol alcohol (200 proof, 10.0% transcutol, and (C) 1.25% HEC, 250 HHX; 1121-8D (gel) containing: (A) 46.9% sodium acetate buffer, 0.10% EDTA disodium, and (B) 20.0% propylene glycol, 1.0% polysorbate 20, 30.0% ethanol alcohol (200 proof, and (C) 1.25% HEC, 250HHX; 1121-10A (lotion) containing: (A) 61.25% acetate buffer pH6.0, 0.05% EDTA sodium, and (B) 20.0% propylene glycol, 0.17% methylparaben, 0.03% propylparaben, and (C) 0.5% HEC, 250 HHX, and (D) 2.0% cetyl alcohol, 5.0% light mineral oil, 7.5% stearyl alcohol, 1.0% polysorbate 20, and 2.5% sorbitan monosteate; 1121-11 (lotion) containing: (A) 1.5% benzyl alcohol, 0.01% citric acid, 86.49% purified water, and (B) 0.50% HEC, 250 HHX, and (C) 5.0% stearyl alcohol, 3.0% mineral oil, 1.25% brij 72, and 2.25% brij 721; 1121-12 (lotion) containing: (A) 1.5% benzyl alcohol, 88.0% purified water, and (B) 0.50% HEC, 250 HHX, and (C) 2.0% stearyl alcohol, 5.0% mineral oil, 2.0% brij 72, and 1.0% brij 721; 1121-13 (lotion) containing: (A) 10.0% propylene glycol, 0.15% methylparaben, 0.05% propylparaben, 74.8% purified water, and (B) 10.0% emulsifying wax, and 5.0% light mineral oil; 1121-14 (lotion) containing: (A) 66.15% purified water, 0.05% EDTA sodium, and (B) 20.0% propylene glycol, 0.17% methylparaben, 0.03% propylparaben, 0.5% HEC, 250 HHX, and (C) 1.0% cetyl alcohol, 5.0% light mineral oil, 5.0% stearyl alcohol, 0.8% brij 72, and 1.5% brij721; 1121-16 (lotion) containing: (A) 10.0% propylene glycol, 0.15% methylparaben, 0.05% propylparaben, 78.3% purified water, and (B) 6.0% emusifying wax, 2.0% stearyl alcohol, 1.0% cetyl alcohol, and 2.5% IPM; 1121-17 (lotion) containing: (A) 0.17% methylparaben, 0.03% propylparaben, 0.05% EDTA disodium, 70.05% purified water, 20.0% propylene glycol, and (B) 0.4% HEC, 250 HHX, and (C) 2.5% stearyl alcohol, 1.5% cetyl alcohol, 2.5% isopropyl myristate, 1.0% brij 72, and 1.8%brij 721; 1121-20(A) (cream) containing: (A) 10.0% propylene glycol, 0.15% methylparaben, 0.05% propylparaben, 0.1% EDTA disodium, and (B) 0.2% poloxamer 188, 78.0% acetate buffer, 0.05% vitamin E TPGS, and (C) 6.0% emusifying wax, 2.0% stearyl alcohol, 1.0% cetyl alcohol, and 2.5% isopropyl myristate (IPM); 1121-20(B) (cream) containing: (A) 10.0% propylene glycol, 0.15% methylparaben, 0.05% propylparaben, 0.1% EDTA disodium, (B) 0.2% poloxamer 188, and 76.6% acetate buffer, and (C) 6.0% emusifying wax, 2.0% stearyl alcohol, 1.0% cetyl alcohol, 2.5% isopropyl myristate (IPM), and 1.0% stearic acid; 1121-20(C) (lotion), (A) 10.0% propylene glycol, 0.15% methylparaben, 0.05% propylparaben, 0.1% EDTA disodium, (B) 0.2% poloxamer 188, and 73.0% acetate buffer, and (C) 6.0% emusifying wax, 2.0% stearyl alcohol, 1.0% cetyl alcohol, 2.5% isopropyl myristate (IPM), and 5.0 oleyl alcohol. These formulations were tested for physical stability.

Additional compositions (e.g., formulations) were prepared, including for example, the following formulations containing the listed ingredients given in weight percentages [w/w (g)] based on the total weight of the composition: 1121-18A containing: (A) 0.05% XMP.629 acetate, and (B) 57.12% sodium acetate buffer (pH 6.0), and 0.15% EDTA disodium, dihydrate, and (C) 10.0% propylene glycol, 0.78% sodium chloride, 0.005% benzalkonium chloride, 0.2% poloxamer 333, and (D) 30.0% ethanol alcohol, 190 proof, and 1.25% HEC, 250 HHX; 1121-18B containing: (A) 0.05% XMP.629 acetate, and (B) 47.12% sodium acetate buffer (pH 6.0), and 0.15% EDTA disodium, dihydrate, and (C) 20.0% propylene glycol, 0.78% sodium chloride, 0.005% benzalkonium chloride, 0.2% poloxamer 333, and (D) 30.0% ethanol alcohol, 190 proof, and 1.25% HEC, 250 HHX; 1121-18C: See Table 13; 1121-18D containing: (A) 0.05% XMP.629 acetate, and (C) 10.0% glycerin, 10.0% glyceryl monooleate, 0.2% poloxamer 333, and (D) 77.75% ethanol alcohol, 190 proof, and 1.8% HPC; 1121-22A: see Table 13; 1121-22B (A) 10.0% propylene glycol, 0.15% EDTA disodium, and (B) 0.2% poloxamer 333, 76.8% acetate buffer, 0.005% benzalkonium chloride, 0.05% XMP.629 acetate, and (D) 6.0% emulsifying wax, 2.0% stearyl alcohol, 1.0% cetyl alcohol, 2.5% isopropyl myristate, 1.0% stearic acid, 0.05% vitamin E TPGS, and (E) 0.25% trolamine; 1121-22C (A) 20.0% propylene glycol, 0.15% EDTA disodium, and (B) 0.2% poloxamer 333, 68.05% acetate buffer, 0.005% benzalkonium chloride, and 0.05% XMP.629 acetate, and (D) 6.0% emulsifying wax, 2.0% stearyl alcohol, 1.0% cetyl alcohol, 2.5% isopropyl myristate, and 0.05% vitamin E TPGS; 1121-25A: see Table 13; 1121-25B: see Table 13; 1121-25C containing: (A) 0.05% XMP.629 acetate, and (B) 48.55% sodium acetate buffer (pH 6.0), 0.15% EDTA disodium, dihydrate, and (C) 20.0% propylene glycol, and 30.0% ethanol alcohol, 200 proof, and (D) 1.25% HEC, 250 HHX; 1121-25D containing: (A) 0.05% XMP.629 acetate, and (B) 48.0% sodium acetate buffer (pH 6.0), 0.15% EDTA disodium, dihydrate, and (C) 20.0% propylene glycol, 30.0% ethanol alcohol, 200 proof, and (D) 1.8% HPC; 1121-27A containing: (A) 0.05% XMP.629 acetate, and (B) 10.0% glycerin, 0.2% poloxamer.333, 87.75% ethanol alcohol, 190 proof, and (C) 1.8% HPC; 1121-27B containing: (A) 0.05% XMP.629 acetate, and (B) 10.0% glycerin, 10.0% glyceryl monooleate, 77.95% ethanol alcohol, 190 proof, and (C) 1.8% HPC; 1121-27C containing: (A) 0.05% XMP.629 acetate, and (B) 10.0% glycerin, 10.0% glyceryl monooleate, 0.2% poloxamer 333, 77.75% ethanol alcohol, 200 proof, and (C) 1.8% HPC; 1121-32A containing: (B) 51.505% sodium acetate buffer (pH 6.0), 0.15% EDTA disodium, dihydrate, and (C) 20.0% propylene glycol, and 0.005% benzalkonium chloride, 30.0% ethanol alcohol, 200 proof, and (D) 1.25% HEC, 250 HHX; 1121-32B containing: (B) 48.395% sodium acetate buffer (pH 6.0), 0.15% EDTA disodium, dihydrate, and (C) 20.0% propylene glycol, 0.005% benzalkonium chloride, 0.2% poloxamer 333, and 30.0% ethanol alcohol, 200 proof, and (D) 1.25% HEC, 250 HHX; 1121-32C containing: (B) 48.6% sodium acetate buffer (pH 6.0), 0.15% EDTA disodium, dihydrate, and (C) 20.0% propylene glycol, 30.0% ethanol alcohol, 200 proof, and (D) 1.25% HEC, 250 HHX; 1121-32D containing: (B) 48.05% sodium acetate buffer (pH 6.0), 0.15% EDTA disodium, dihydrate, and (C) 20.0% propylene glycol, 30.0% ethanol alcohol, 200 proof, and (d) 1.8% HPC; 1121-33A containing: (A) 20.0% propylene glycol, 0.15% EDTA disodium, and (B) 70.55% acetate buffer, 0.005% benzalkonium chloride, and (C) 0.4% HEC, 250 HHX, and (D) 2.5% stearyl alcohol, 1.5% cetyl alcohol, 2.5% isopropyl myristate, 1.0% brij 72, and 1.8 brij 721; 1121-33B (A) 10.0% propylene glycol, 0.15% EDTA disodium, and (B) 0.2% poloxamer 333, 77.25% acetate buffer, 0.005% benzalkonium chloride, and (D) 6.0% emulsifying wax, 2.0% stearyl alcohol, 1.0% cetyl alcohol, 2.5% isopropyl myristate, 1.0% stearic acid, 0.05% vitamin E TPGS, and (E) 0.25% trolamine; 1121-33C containing: (A) 20.0% propylene glycol, 0.15% EDTA disodium, and (B) 0.2% poloxamer 333, 68.5% acetate buffer, 0.005% benzalkonium chloride, and (D) 6.0% emulsifying wax, 2.0% stearyl alcohol, 1.0% cetyl alcohol, 2.5% isopropyl myristate, and 0.05% vitamin E TPGS; 1121-38A containing: (B) 77.595% sodium acetate buffer (pH 6.0), 0.15% EDTA disodium, dihydrate, and (C) 20.0% propylene glycol, 0.005% benzalkonium chloride, 0.2% poloxamer 333, and (D) 1.25% HEC, 250 HHX; 1121-38B (B) 48.395% sodium acetate buffer (pH 6.0), 0.15% EDTA disodium, dihydrate, and (C) 20.0% propylene glycol, 0.005% benzalkonium chloride, 0.2% poloxamer 333, 30.0% ethanol alcohol, 200 proof, and (D) 1.25% HEC, 250 HHX; 1121-39 containing: (A) 20.0% propylene glycol, and 0.15% EDTA disodium, and (B) 70.145% acetate buffer, 0.005% benzalkonium chloride, and (C) 0.4% HEC, 250 HHX, and (D) 2.5% stearyl alcohol, 1.5% cetyl alcohol, 2.5% isopropyl myristate, 1.0% brij 72, and 1.8% brij 721; 1121-41A: see Table 14; 1121-41B: see Table 14; 1121-41C: see Table 14; 1121-43A: see Table 14; 112143B: see Table 14; 1121-77A: see Table 15; 1121-77B: see Table 15; 1121-77C: see Table 15; 1121-77D: see Table 15; 1121-77E: see Table 15; 1121-77F: see Table 15; 1121-80: see Table 15; 1121-85A containing: (A) 0.052% XMP.629 acetate, and (B) 97.308% lOmM sodium acetate buffer (pH 6.0), 0.15% EDTA disodium, dihydrate, and (C) 0.78% sodium chloride, 0.01% benzalkonium chloride, and 0.20% poloxamer 333, and (D) 0.25% lOmM sodium acetate buffer (pH 6.0), and (E) 1.25% HEC, 250 HHX; 1121-85B containing: (A) 0.052% XMP.629 acetate, and (B) 97.308% 10 mM sodium acetate buffer (pH 6.0), 0.15% EDTA disodium, dihydrate, and (C) 0.78% sodium chloride, 0.01% benzalkonium chloride, and 0.20% poloxamer 333, and (D) 0.25% 10 mM sodium acetate buffer (pH 6.0), and (E) 1.50% HEC, 250 HHX; 1121-85C (A) 0.052% XMP.629 acetate, and (B) 96.308% 10 mM sodium acetate buffer (pH 6.0), 0.15% EDTA disodium, dihydrate, and (C) 1.0% propylene glycol, 0.78% sodium chloride, 0.1% benzalkonium chloride, and 0.20% poloxamer 333, and (D) 0.25% 10 mM sodium acetate buffer (pH 6.0), and (E) 1.25% HEC, 250 HHX; 1121-85D (A) 0.052% XMP.629 acetate, and (B) 96.308% 10 mM sodium acetate buffer (pH 6.0), 0.15% EDTA disodium, dihydrate, and (C) 1.0% propylene glycol, 0.78% sodium chloride, 0.01% benzalkonium chloride, and 0.20% poloxamer 333, and (E) 1.50% HEC, 250 HHX; 1121-85E (A) 0.052% XMP.629 acetate, and (B) 95.308% 10 mM sodium acetate buffer (pH 6.0), 0.15% EDTA disodium, dihydrate, and (C) 2.0% propylene glycol, 0.78% sodium chloride, 0.01% benzalkonium chloride, and 0.20% poloxamer 333, and (D) 0.25% 10 mM sodium acetate buffer (pH 6.0) and (E) 1.25% HEC, 250 HHX; 1121-85F (A) 0.052% XMP.629 acetate, and (B) 95.308% 10 mM sodium acetate buffer (pH 6.0), 0.15% EDTA disodium, dihydrate, and (C) 2.0% propylene glycol, 0.78% sodium chloride, 0.01% benzalkonium chloride, and 0.20% poloxamer 333, and (E) 1.50% HEC, 250 HHX; 1121-85G (A) 0.052% XMP.629 acetate, and (B) 92.308% lOmM sodium acetate buffer (pH 6.0), 0.15% EDTA disodium, dihydrate, and (C) 5.0% propylene glycol, 0.78% sodium chloride, 0.01% benzalkonium chloride, and 0.20% poloxamer 333, and (E) 1.50% HEC, 250 HHX; 1121-87A containing: (B) 97.36% 10 mM sodium acetate buffer (pH 6.0), 0.15% EDTA disodium, dihydrate, and (C) 0.0% propylene glycol, 0.78% sodium chloride, 0.01% benzalkonium chloride, and 0.20% poloxamer 333, and (D) 0.25% 10 mM sodium acetate buffer (pH 6.0) and (E) 1.25% HEC, 250 HHX; 1121-87B containing: (B) 97.36% 10 mM sodium acetate buffer (pH 6.0), 0.15% EDTA disodium, dihydrate, and (C) 0.0% propylene glycol, 0.78% sodium chloride, 0.01% benzalkonium chloride, and 0.20% poloxamer 333, and (E) 1.50% HEC, 250 HHX; 1121-87C containing: (B) 96.36% 10 mM sodium acetate buffer (pH 6.0), 0.15% EDTA disodium, dihydrate, and (C) 1.0% propylene glycol, 0.78% sodium chloride, 0.01% benzalkonium chloride, and 0.20% poloxamer 333, and (D) 0.25% 10 mM sodium acetate buffer (pH 6.0) and (E) 1.25% HEC, 250 HHX; 1121-87D containing: (B) 96.36% 10 mM sodium acetate buffer (pH 6.0), 0.15% EDTA disodium, dihydrate, and (C) 1.0% propylene glycol, 0.78% sodium chloride, 0.01% benzalkonium chloride, and 0.20% poloxamer 333, and (E) 1.50% HEC, 250 HHX; 1121-87E containing: (B) 95.36% 10 mM sodium acetate buffer (pH 6.0), 0.15% EDTA disodium, dihydrate, and (C) 2.0% propylene glycol, 0.78% sodium chloride, 0.01% benzalkonium chloride, and 0.20% poloxamer 333, and (D) 0.25% 10 mM sodium acetate buffer (pH 6.0) and (E) 1.25% HEC, 250 HHX; 1121-87F containing: (B) 95.36% 10 mM sodium acetate buffer (pH 6.0), 0.15% EDTA disodium, dihydrate, and (C) 2.0% propylene glycol, 0.78% sodium chloride, 0.01% benzalkonium chloride, and 0.20% poloxamer 333, and (E) 1.50% HEC, 250 HHX; 1121-87G containing: (B) 92.36% 10 mM sodium acetate buffer (pH 6.0), 0.15% EDTA disodium, dihydrate, and (C) 5.0% propylene glycol, 0.78% sodium chloride, 0.01% benzalkonium chloride, and 0.20% poloxamer 333, and (E) 1.50% HEC, 250 HHX. These formulations were tested for antimicrobial effectiveness, skin penetration, stability and/or assay method development.

Additional compositions (e.g., formulations) were prepared, including for example, the following formulations containing the listed ingredients given in weight percentages [w/w (g)] based on the total weight of the composition: 1121-48A containing: (A) 0.05% XMP.629 acetate, and (B) 97.565% 10 mM sodium-acetate buffer (pH 6.0), 0.15% EDTA disodium, dihydrate, and (C) 0.0% propylene glycol, 0.78% sodium chloride, 0.005% benzalkonium chloride, 0.20% poloxamer 333, and (D) 1.25% HEC, 250 HHX; 112148B containing: (A) 0.05% XMP.629 acetate, and (B) 92.565% 10 mM sodium-acetate buffer (pH 6.0), 0.15% EDTA disodium, dihydrate, and (C) 5.0% propylene glycol, 0.78% sodium chloride, 0.005% benzalkonium chloride, 0.20% poloxamer 333, and (D) 1.25% HEC, 250 HHX; 1121-48C containing: (A) 0.05% XMP.629 acetate, and (B) 87.565% 10 mM sodium-acetate buffer (pH 6.0), 0.15% EDTA disodium, dihydrate, and (C) 10.0% propylene glycol, 0.78% sodium chloride, 0.005% benzalkonium chloride, 0.20% poloxamer 333, and (D) 1.25% HEC, 250 HHX; 1121-92A containing: (A) 95.48% purified water, USP, 0.005% acetic acid solution (36%), 0.13% sodium acetate trihydrate, 0.15% EDTA disodium, dihydrate, and (B) 2.0% propylene glycol, 0.78% sodium chloride, 0.2% poloxamer 333, and (C) 0.0025% BAK, and (D) 1.25% HEC, 250 HHX; 1121-92B containing: (A) 95.48% purified water, USP, 0.005% acetic acid solution (36%), 0.13% sodium acetate trihydrate, 0.15% EDTA disodium, dihydrate, and (B) 2.0% propylene glycol, 0.78% sodium chloride, 0.2% poloxamer 333, and (C) 0.004% BAK, and (D) 1.25% HEC, 250 HHX; 1121-92C containing: (A) 95.48% purified water, USP, 0.005% acetic acid solution (36%), 0.13% sodium acetate trihydrate, 0.15% EDTA disodium, dihydrate, and (B) 2.0% propylene glycol, 0.78% sodium chloride, 0.2% poloxamer 333, and (C) 0.006% BAK, and (D) 1.25% HEC, 250 HHX. These formulations were tested for antimicrobial effectiveness.

Additional compositions (e.g., formulations) were prepared, including for example, the following formulations containing the listed ingredients given in weight percentages [w/w (g)] based on the total weight of the composition: 1121-45A containing: (A) 0.01% XMP.629 acetate, and (B) 77.605% 10 mM sodium-acetate buffer (pH 6.0), 0.15% EDTA disodium, dihydrate, and (C) 20.0% propylene glycol, 0.78% sodium chloride, 0.005% benzalkonium chloride, 0.2% poloxamer 333, and (D) 1.25% HEC, 250 HHX; 1121-45B containing: (A) 0.10% XMP.629 acetate, and (B) 77.515% 10 mM sodium-acetate buffer (pH 6.0), 0.15% EDTA disodium, dihydrate, and (C) 20.0% propylene glycol, 0.78% sodium chloride, 0.005% benzalkonium chloride, 0.2% poloxamer 333, and (D) 1.25% HEC, 250 HHX; 1121-69A containing: (B) 97.365% 10 mM sodium-acetate buffer (pH 6.0), 0.15% EDTA disodium, dihydrate, and (C) 0.78% sodium chloride, 0.005% benzalkonium chloride, 0.20% poloxamer 333, and (D) 1.50% HEC, 250 HHX; 1121-69B containing: (B) 87.365% 10 mM sodium-acetate buffer (pH 6.0), 0.15% EDTA disodium, dihydrate, and (C) 10.0% propylene glycol, 0.78% sodium chloride, 0.005% benzalkonium chloride, 0.20% poloxamer 333, and (D) 1.50% HEC, 250 HHX; 1121-71A containing: (A) 0.0104% XMP.629 acetate, and (B) 97.3546% 10 mM sodium-acetate buffer (pH 6.0) (pH 6.0), 0.15% EDTA disodium, dihydrate, and (C) 0.78% sodium chloride, 0.005% benzalkonium chloride, 0.20% poloxamer 333, and (D) 1.50% HEC, 250 HHX; 1121-71B containing: (A) 0.0104% XMP.629 acetate, and (B) 87.3546% 10 mM sodium-acetate buffer (pH 6.0), 0.15% EDTA disodium, dihydrate, and (C) 10.0% propylene glycol, 0.78% sodium chloride, 0.005% benzalkonium chloride, 0.20% poloxamer 333, and (D) 1.50% HEC, 250 HHX; 1121-73A containing: (A) 0.052% XMP.629 acetate, and (B) 97.313% 10 mM sodium-acetate buffer (pH 6.0), 0.15% EDTA disodium, dihydrate, and (C) 0.78% sodium chloride, 0.005% benzalkonium chloride, 0.20% poloxamer 333, and (D) 1.50% HEC, 250 HHX; 1121-73B containing: (A) 0.052% XMP.629 acetate, and (B) 87.313% 10 mM sodium-acetate buffer (pH 6.0), 0.15% EDTA disodium, dihydrate, and (C) 10.0% propylene glycol, 0.78% sodium chloride, 0.005% benzalkonium chloride, 0.20% poloxamer 333, and (D) 1.50% HEC, 250 HHX; 1121-75A containing: (A) 0.0104% XMP.629 acetate, and (B) 97.261% 10 mM sodium-acetate buffer (pH 6.0), 0.15% EDTA disodium, dihydrate, and (C) 0.78% sodium chloride, 0.005% benzalkonium chloride, 0.20% poloxamer 333, and (D) 1.50% HEC, 250 HHX; 1121-75B containing: (A) 0.0104% XMP.629 acetate, and (B) 87.261% 10 mM sodium-acetate buffer (pH 6.0), 0.15% EDTA disodium, dihydrate, and (C) 10.0% propylene glycol, 0.78% sodium chloride, 0.005% benzalkonium chloride, 0.20% poloxamer 333, and (D) 1.50% HEC, 250 HHX; 1121-83A containing: (B) 97.365% 10 mM sodium-acetate buffer (pH 6.0), 0.15% EDTA disodium, dihydrate, and (C) 0.78% sodium chloride, 0.005% BAK, 0.20% poloxamer 333, and (D) 1.50% HEC, 250 HHX; 1121-83B containing: (B) 87.365% 10 mM sodium-acetate buffer (pH 6.0), 0.15% EDTA disodium, dihydrate, and (C) 10.0% propylene glycol, 0.78% sodium chloride, 0.005% BAK, 0.20% poloxamer 333, and (D) 1.50% HEC, 250 HHX; 1121-84A containing: (B) 97.37% 10 mM sodium-acetate buffer (pH 6.0), 0.15% EDTA disodium, dihydrate, and (C) 0.78% sodium chloride, 0.20% poloxamer 333, and (D) 1.50% HEC, 250 HHX; 1121-84B containing: (B) 87.37% 10 mM sodium-acetate buffer (pH 6.0), 0.15% EDTA disodium, dihydrate, and (C) 10.0% propylene glycol, 0.78% sodium chloride, 0.20% poloxamer 333, and (D) 1.50% HEC, 250 HHX. These formulations were tested for analytical method development and/or validation.

Additional compositions (e.g., formulations) were prepared, including for example, the following formulations containing the listed ingredients given in weight percentages [w/w (g)] based on the total weight of the composition: 1121-54A containing: 0.06% acetic acid 99.94% purified water; 1121-54B containing: 0.082% sodium-acetate 99.918% purified water; 1121-54C containing: 2.95% acetic acid soln. 97.05% sodium-acetate solution; 1121-58A containing: (A) 97.365% 10 mM sodium acetate buffer 0.15% EDTA disodium dihydrate, and (B) 0.78% sodium chloride 0.005% benzalkonium chloride 0.2% poloxamer 333, and (C) 1.5% HEC, 250 HNX; 1121-58B containing: (A) 97.115% 10 mM sodium acetate buffer 0.15% EDTA, and (B) 0.78% sodium chloride 0.005% benzalkonium chloride, and 0.2% poloxamer 333, and (C) 1.75% HEC, 250 HNX; 1121-59A containing: (A) 96.365% 10 mM sodium acetate buffer 0.15% EDTA, and (B) 1.0% propylene glycol 0.78% sodium chloride 0.005% benzalkonium chloride 0.2% poloxamer 333, and (C) 15% HEC, 250 HNX; 1121-59B containing: (A) 92.365% 10 mM sodium acetate buffer 0.15% EDTA, and (B) 5.0% propylene gycol 0.78% sodium chloride 0.005% benzalkonium chloride 0.2% poloxamer 333, and 1.5% HEC, 250 HMX; 1121-60A containing: (A) 92.15% 10 mM sodium acetate buffer 0.15% EDTA, and (B) 5.0% propylene glycol 0.78% sodium chloride 0.005% Benzalkonium chloride 0.2% poloxamer 333, and (C) 1.75% HEC, 250 HNX. These formulations were tested for physical evaluation, user focus group evaluation and/or microbial limits testing.

In vitro percutaneous absorption studies using various formulations of (³H)-labeled XMP.629 on dermal samples were conducted to characterize the absorption properties of XMP.629. It has been shown that high in vitro skin penetration along with high skin levels of drug correlate with in vivo cutaneous efficacy following topical application. It has also been shown that systemic exposure, i.e., the rate and extent of drug transit through the skin, can be modified by changing the concentration of an active ingredient in particular formulations. Additionally, the use of excipients can also modify the amount of active moiety in the skin versus the amount that penetrates through the skin. Using a variety of formulations, studies were performed with varying amounts of XMP.629 and excipients to determine the degree of penetration into and through the skin.

In an initial skin penetration study, four formulations (1121-18C, 1121-25A, 1121-25B, 1121-22A) were tested. Based on formula 1121-18C, five additional formulations (1121-41A, 1121-41B, 1121-41C, 1121-43A and 1121-43B) containing different amounts of PG were prepared for an additional skin penetration study. The in-vitro skin study indicated that formulations containing different PG levels had similar skin deposition and penetration of XMP.629 acetate. The formulations showed very favorable amounts of drug penetration into and through the skin.

The effect of PG and HEC (250 HHX) concentration on skin penetration of the drug was further explored in an additional penetration study. Formulations containing 0%, 1%, 2% and 5% PG, and 1.25% and 1.5% HEC 250 HHX were prepared. The skin penetration study showed that varying the concentration of PG in the aqueous gel formulation did not significantly modify the rate and extent of drug transit through the skin. The highest drug deposition in the skin was from the tested formulation devoid of PG. Additionally, there was a trend for increased epidermal deposition with lower HEC concentration.

Since the formulations showed favorable amounts of drug penetration through skin, results from antimicrobial activity tests, stability tests and physical evaluation tests were also evaluated.

A preferred formulation was 1121-77E containing 2% PG (see also, Table 12).

In an initial series of studies, four formulations, containing components listed in Table 13 below, were prepared and tested. In another series of studies, five additional formulations containing components listed in Table 14 below, including varying concentrations (0-20%) of propylene glycol, were prepared and tested. In yet another series of studies, seven additional formulations containing components listed in Table 15 below, including varying concentrations of propylene glycol (0-5%) and of hydroxyethylcellulose (HEC) (1.25 or 1.5%), were prepared and tested.

TABLE 13

| | | | Alcoholic | |
|---|---|---|---|---|
| | Aqueous Gel | | Gel | |
| | (Poloxamer | Alcoholic | (Poloxamer | Aqueous |
| | 333) | Gel | 333) | Lotion |
| Ingredient (% w/w) | 1121-18C | 1121-25A | 1121-25B | 1121-22A |
| XMP.629 | 0.05 | 0.05 | 0.05 | 0.05 |
| Sodium-acetate | 77.565 | 51.455 | 48.345 | 70.095 |
| buffer (pH 6.0) | | | | |
| EDTA disodium, | 0.15 | 0.15 | 0.15 | 0.15 |
| dehydrate | | | | |
| Propylene Glycol | 20.0 | 20.0 | 20.0 | 20.0 |
| Sodium Chloride | 0.78 | — | — | — |
| Benzalkonium | 0.005 | 0.005 | 0.005 | 0.005 |
| Chloride | | | | |
| Poloxamer 333 | 0.2 | — | 0.2 | — |
| (pluronic P-103) | | | | |
| Ethanol Alcohol, | — | 30.0 | 30.0 | — |
| 200 proof | | | | |
| HEC, 250 HHX | 1.25 | 1.25 | 1.25 | 0.4 |
| Stearyl Alcohol | — | — | — | 2.5 |
| Cetyl Alcohol | — | — | — | 1.5 |
| Isopropyl | — | — | — | 2.5 |
| Myristate | | | | |
| Brij 72 | — | — | — | 1.0 |
| Brij 721 | — | — | — | 1.8 |

TABLE 14

| | Formulation Batch Names | | | | |
|---|---|---|---|---|---|
| Ingredient (% w/w) | 1121-41(A) | 1121-41(B) | 1121-41(C) | 1121-43(A) | 1121-43(B) |
| XMP.629 | 0.05 | 0.05 | 0.05 | 0.05 | 0.05 |
| Sodium acetate buffer (pH 6.0) | 97.565 | 92.565 | 87.565 | 82.565 | 77.565 |
| EDTA disodium, dehydrate | 0.15 | 0.15 | 0.15 | 0.15 | 0.15 |
| Propylene glycol | 0 | 5.0 | 10.0 | 15.0 | 20.0 |
| Sodium chloride | 0.78 | 0.78 | 0.78 | 0.78 | 0.78 |
| Benzalkonium chloride | 0.005 | 0.005 | 0.005 | 0.005 | 0.005 |
| Poloxamer 333 (pluronic P-103) | 0.2 | 0.2 | 0.2 | 0.2 | 0.2 |
| HEC, 250 HHX | 1.25 | 1.25 | 1.25 | 1.25 | 1.25 |

TABLE 15

| | Formulation ID: | | | | | | |
|---|---|---|---|---|---|---|---|
| | A | B | C | D | E | F | G |
| Ingredient (% w/w) | 1121-77A | 1121-77B | 1121-77C | 1121-77D | 1121-77E | 1121-77F | 1121-80 |
| XMP.629 | 0.05 | 0.05 | 0.05 | 0.05 | 0.05 | 0.05 | 0.05 |
| Sodium-acetate buffer (pH | 97.56 | 97.31 | 96.56 | 96.31 | 95.56 | 95.31 | 92.31 |
| 6.0) | | | | | | | |
| EDTA disodium, | 0.15 | 0.15 | 0.15 | 0.15 | 0.15 | 0.15 | 0.15 |
| dehydrate | | | | | | | |
| Propylene Glycol | 0 | 0 | 1 | 1 | 2 | 2 | 5 |
| Sodium Chloride | 0.78 | 0.78 | 0.78 | 0.78 | 0.78 | 0.78 | 0.78 |
| Benzalkonium Chloride | 0.01 | 0.01 | 0.01 | 0.01 | 0.01 | 0.01 | 0.01 |
| Poloxamer 333 (pluronic | 0.2 | 0.2 | 0.2 | 0.2 | 0.2 | 0.2 | 0.2 |
| P-103) | | | | | | | |
| HEC, 250 HHX | 1.25 | 1.5 | 1.25 | 1.5 | 1.25 | 1.5 | 1.5 |

For the penetration studies, epidermal samples excised from human abdominal skin of a donor, were assembled onto Franz static diffusion cells (Crown Bio Scientific, Clinton, N.J.) with a 15 mm diameter orifice and O-ring joint that were mounted on 9-cell manifolds and maintained at a temperature of 32° C. by use of recirculating water baths. The diffusion cells have an opening with a nominal area of 1.767 cm² and a receptor compartment with a volume ranging between 12 to 14 mL (in these experiments generally a 13.0-13.5 mL volume was used). Each diffusion cell was assembled by placing the excised human abdominal skin dermal-side down and then a Teflon® O-ring (which rested in the groove of the receptor side, bottom half, of the diffusion cell). The donor side, top half, of the diffusion cell was then placed on top of the O-ring resting on the skin and held in place by use of a pinch clamp. The joint between the donor and receptor compartments of each diffusion cell was wrapped with Parafilme to prevent evaporation of the receptor solution. Each diffusion cell was filled with receptor solution warmed to 32° C. consisting of degassed PBS with 0.1% sodium azide and 1.5% Oleth-20 taking care to dispel any air bubbles from under the skin and with continued filling until the receptor solution level on the injection port was level with the membrane. The receptor fluid was continuously stirred using a Teflon® magnetic stir bar and an inoculating loop cut to ˜5.0 cm or alternatively ˜3.5 cm from the top of the loop. The skin was allowed to equilibrate with the receptor solution for 1 hour prior to application of a representative XMP.629 formulation.

Representative XMP.629 formulations, for example, as listed in Tables 13-15, were applied on dermal samples as follows. Spike formulations with radiolabeled 3H-XMP.629 were prepared to achieve a radiolabeled concentration of approximately 1.0 μCi/dose. In a glass v-vial, 20 μl (≈20 μCi) of stock ³H-XMP.629 was dispensed. Under a steady stream of nitrogen or argon gas in the hood, most of the solvent was evaporated without evaporating to dryness. A total of 170 mg of formulation base was added to the evaporated samples in 3 portions (56.3 mg/portion). Between each portion, the spiked sample was mixed manually with a positive displacement pipette tip and centrifuge. The manual mixing and centrifuging were performed several times to ensure homogeneity. Homogeneity of ³H-radiolabel in the formulations was confirmed with 4 mg of formulation and 5 standards per formulation with 4 mg of formulation were prepared. A dose (5 mg/cm² or ˜8.9 mg formulation per dose) of a representative XMP.629 formulation was applied using a positive displacement pipet and spread on to the skin on the diffusion cells prepared as described above. Each formulation tested was applied in an alternating fashion to 5 diffusion cells.

At 3, 20, and 24 hours after dosing, the receptor fluid (˜6.0 mL) from each diffusion cell was collected through the sampling port using a syringe fitted with Teflon tubing on the needle and then replaced with fresh receptor fluid maintained at 32° C. The cells were carefully inverted to remove air bubbles. The collected receptor solution samples were placed in clean scintillation vials and their weight was recorded. To each vial, 10 mL of Ready Gel®, Bio-Rad, Hercules, Calif. was added and the samples were shaken on a platform rocker until a gel was formed. Following the 24-hour exposure period, the skin was wiped twice consecutively with a dry cotton swab. Each swab was placed in a separate scintillation vial and 10 mL of Ready Value® Bio-Rad, Hercules, Calif. was added. The samples were shaken on the platform rocker and allowed to sit overnight. The receptor solution remaining after the 24 hour receptor solution was collected and placed in a scintillation vial. The weight of the receptor solution was recorded. Cell caps were removed and placed in individual 50 mL disposable beakers. Each cell cap was soaked in 95% ethanol (EtOH) for at least 3 hours and wiped with one dry swab. For each cap, the EtOH wash and the corresponding swab were pooled. Ten milliliters of Ready Value® was added. Residual formulation was removed from the stratum corneum with one cellophane tape-strip that was then dissolved overnight in 4 mL tetrahydrofuran (THF). Ten milliliters of Ready Value® was added to the digested tape-strip. The epidermis was physically separated from the dermis and each component was separately solubilized in 2 mL of 2N KOH. After solubilization, 2.5 mL tissue neutralizing solution, 5 mL deionized water, and 10 mL Ready Gel®, were added to the solubilized skin components. The samples were shaken on a platform shaker until a gel was formed.

All receptors, wipes, tape-strips, epidermis, dermis, and cell cap wash samples were analyzed for ³H radioactivity by liquid scintillation counting using Ready Gel® for receptor and tissue samples and Ready Value® for all other samples prepared as described above. The percent of the applied dose in each of the these samples was calculated. The dose recovered from each diffusion cell was calculated as the summation of the percent of the applied dose in each of the above samples.

Results illustrating the rate and extent of drug penetration with various XMP.629 formulations are shown in Tables 16, 18 and 20, where the cumulative percent of the applied dose and time of receptor fluid sample collection are shown. Additional results of the percent of the applied dose in the tape-strip, epidermis (viable and nonviable), dermis, and receptor fluid following 24 hours of exposure, along with total dose recovered for each formulation are shown in Tables 17, 19 and 21 as follows.

Results from the formulations of XMP.629 listed in Table 13 showed that skin penetration of the four tested formulations ranged from 3.9 to 5.3 per cent of the applied dose as presented in Table 16. Epidermal levels (includes most of the stratum corneum) post tape-strip ranged from 6.6% to 15% of the applied dose and the highest levels were demonstrated with the Aqueous Gel formulation and the Aqueous Lotion formulation, 14% and 15% of the applied dose, respectively, as shown in Table 17. The highest dermal levels were obtained with the Aqueous gel, 1.0% of the applied dose as shown in Table 17. Dermal levels ranged from 0.25% to 1.0% of the applied dose. Dose recovery was low and variable, possibly due to binding of the drug to the cotton swabs used to remove formulation from the skin surface at 24-hours.

TABLE 16
A) Aqueous gel, batch 1121-18C

| | | | Total cell |
|---|---|---|---|
| Time | Cell | Dose (mg) | volume (g) |
| 0 hours | A1 | 10.4 | 12.3947 |
| | A2 | 8.4 | 12.9660 |
| | A3 | 8.9 | 12.7471 |
| | D4 | 9.8 | 12.6206 |
| | D5 | 8.7 | 12.3185 |

| | | Receptor | Cumu- | | | |
|---|---|---|---|---|---|---|
| | | Collected | lative | Cumulative | | |
| Time | Cell | (g) | % dose | Mean | stdev | % CV |
| 3 hours | A1 | 6.1913 | 0.8 | | | |
| | A2 | 5.9145 | 1.5 | | | |
| | A3 | 6.1781 | 1.5 | | | |
| | D4 | 6.2799 | 0.6 | | | |
| | D5 | 6.4504 | 1.1 | 1.1 | 0.407 | 36.6% |
| 20 hours | A1 | 6.6562 | 3.3 | | | |
| | A2 | 6.6606 | 4.1 | | | |
| | A3 | 6.5102 | 4.5 | | | |
| | D4 | 6.4182 | 3.1 | | | |
| | D5 | 6.1177 | 3.8 | 3.7 | 0.567 | 15.2% |
| 24 hours | A1 | 6.3651 | 3.4 | | | |
| | A2 | 6.6962 | 4.2 | | | |
| | A3 | 6.7380 | 4.6 | | | |
| | D4 | 6.2808 | 3.1 | | | |
| | D5 | 6.4798 | 3.9 | 3.8 | 0.600 | 15.6 |

B) EtOH gel devoid of poloxamer 333 and sodium
chloride, batch 1121-25A

| | | | Total cell |
|---|---|---|---|
| Time | Cell | Dose (mg) | volume (g) |
| 0 hours | A4 | 9.0 | 12.4168 |
| | A5 | 8.3 | 12.3914 |
| | D1 | 8.4 | 12.5510 |
| | D2 | 8.5 | 12.4645 |
| | D3 | 7.8 | 12.6518 |

| | | Receptor | Cumu- | | | |
|---|---|---|---|---|---|---|
| | | Collected | lative | Cumulative | | |
| Time | Cell | (g) | % dose | Mean | stdev | % CV |
| 3 hours | A4 | 6.2068 | 1.9 | | | |
| | A5 | 6.1644 | 0.7 | | | |
| | D1 | 6.2373 | 1.2 | | | |
| | D2 | 6.4633 | 0.7 | | | |
| | D3 | 6.4544 | 0.7 | 1.0 | 0.528 | 50.4% |
| 20 hours | A4 | 6.2700 | 4.7 | | | |
| | A5 | 6.5736 | 3.9 | | | |
| | D1 | 6.4714 | 4.4 | | | |
| | D2 | 6.7275 | 3.9 | | | |
| | D3 | 6.3730 | 3.0 | 4.0 | 0.627 | 15.7% |
| 24 hours | A4 | 6.7451 | 4.8 | | | |
| | A5 | 6.1469 | 3.9 | | | |
| | D1 | 6.7706 | 4.5 | | | |
| | D2 | 6.4596 | 4.1 | | | |
| | D3 | 6.6064 | 3.1 | 4.1 | 0.628 | 15.4% |

C) EtOH gel with poloxamer 333, batch 1121-25B

| | | | Total cell |
|---|---|---|---|
| Time | Cell | Dose (mg) | volume (g) |
| 0 hours | B1 | 9.5 | 12.8164 |
| | B2 | 9.2 | 12.5906 |
| | C3 | 8.3 | 12.4732 |
| | C4 | 8.9 | 12.6193 |
| | C5 | 8.7 | 12.6076 |

| | | Receptor | Cumu- | | | |
|---|---|---|---|---|---|---|
| | | Collected | lative | Cumulative | | |
| Time | Cell | (g) | % dose | mean | stdev | % CV |
| 3 hours | B1 | 6.5593 | 1.0 | | | |
| | B2 | 6.4188 | 1.0 | | | |
| | C3 | 6.1506 | 1.4 | | | |
| | C4 | 6.1071 | 0.8 | | | |
| | C5 | 6.3183 | 0.9 | 1.0 | 0.239 | 23.5% |
| 20 hours | B1 | 6.7816 | 3.8 | | | |
| | B2 | 6.5305 | 3.9 | | | |
| | C3 | 6.5091 | 4.7 | | | |
| | C4 | 6.4114 | 3.5 | | | |
| | C5 | 6.6883 | 3.8 | 3.9 | 0.468 | 11.9% |
| 24 hours | B1 | 6.6951 | 3.9 | | | |
| | B2 | 6.4345 | 4.0 | | | |
| | C3 | 6.4317 | 4.9 | | | |
| | C4 | 6.6199 | 3.7 | | | |
| | C5 | 6.6076 | 3.9 | 4.1 | 0.487 | 11.9% |

D) Aqueous lotion, batch 1121-22A

| | | | Total cell |
|---|---|---|---|
| Time | Cell | Dose (mg) | volume (g) |
| 0 hours | B3 | 9.4 | 12.8181 |
| | B4 | 8.8 | 12.5875 |
| | B5 | 9.1 | 12.6335 |
| | C1 | 8.4 | 12.7231 |
| | C2 | 8.5 | 12.5977 |

| | | Receptor | Cumu- | | | |
|---|---|---|---|---|---|---|
| | | Collected | lative | Cumulative | | |
| Time | Cell | (g) | % dose | mean | stdev | % CV |
| 3 hours | B3 | 6.2921 | 1.0 | | | |
| | B4 | 6.6830 | 1.0 | | | |
| | B5 | 6.8530 | 0.4 | | | |
| | C1 | 6.6989 | 1.1 | | | |
| | C2 | 6.8959 | 0.9 | 0.9 | 0.278 | 31.0% |
| 20 hours | B3 | 6.6057 | 5.0 | | | |
| | B4 | 6.3955 | 4.5 | | | |
| | B5 | 6.6131 | 4.1 | | | |
| | C1 | 6.3722 | 5.6 | | | |
| | C2 | 6.8629 | 5.2 | 4.9 | 0.594 | 12.2% |
| 24 hours | B3 | 6.6467 | 5.3 | | | |
| | B4 | 6.3649 | 5.0 | | | |
| | B5 | 6.7022 | 4.6 | | | |
| | C1 | 6.2759 | 5.8 | | | |
| | C2 | 6.6479 | 5.5 | 5.3 | 0.493 | 9.4% |

TABLE 17

| | | Single Tape | | | | Dose |
|---|---|---|---|---|---|---|
| Formulation | | Strip | Epidermis | Dermis | Receptor | Recovered |
| A) Aqueous Gel, | Mean | 13.20 | 14.32 | 1.04 | 3.85 | 57.84 |
| batch 1121-18C | SD | 6.08 | 6.94 | 0.92 | 0.60 | 10.32 |
| | % CV | 46.05 | 48.43 | 88.74 | 15.59 | 17.85 |
| B) Alcohol (EtOH) | Mean | 8.53 | 6.63 | 0.43 | 4.08 | 38.93 |
| Gel devoid of | SD | 5.40 | 3.36 | 0.28 | 0.63 | 8.73 |
| poloxamer 333 | % CV | 63.31 | 50.60 | 65.15 | 15.37 | 22.41 |
| and sodium | | | | | | |
| chloride, batch | | | | | | |
| 1121-25A | | | | | | |
| C) Alcohol (EtOH) | Mean | 9.86 | 8.21 | 0.25 | 4.09 | 47.32 |
| Gel with poloxamer | SD | 4.24 | 4.03 | 0.14 | 0.49 | 12.76 |
| 333, | % CV | 42.96 | 49.05 | 53.83 | 11.91 | 26.96 |
| Batch 1121-25B | | | | | | |
| D) Aqueous Lotion, | Mean | 22.33 | 15.43 | 0.41 | 5.25 | 84.33 |
| Batch 1121-22A | SD | 14.18 | 9.49 | 0.31 | 0.49 | 6.82 |
| | % CV | 63.49 | 61.47 | 76.14 | 9.38 | 8.09 |

Note:
Values are in % of Applied Dose;
N = 5 cells per formulation.0

Results from the formulations of XMP.629 listed in Table 14 showed that skin penetration of the four tested formulations ranged from 2.80 to 3.94 percent of the applied dose, as presented in Table 19. These five formulations contained propylene glycol at levels varying from 0% to 20%. Epidermal levels, including most of the stratum corneum, post tape-strip ranged from 15.4% to 22.5% of the applied dose with 5% to 20% propylene glycol as shown in Table 18. The highest epidermal levels were observed with 0% propylene glycol at 48% of the applied dose, which can possibly be attributed to residual formulation being left on the skin. The highest dermal levels (0.47% of the applied dose) were obtained with 0% propylene glycol. Dermal levels ranged from 0.15% to 0.47% of the applied dose as shown in Table 19. Dose recovery ranged from 83% to 100%. These results surprisingly showed that varying the concentration of propylene glycol in the formulation did not significantly modify the rate and extent of drug transit through the skin. In fact, there was an unexpected observation of a trend for higher drug deposition in the skin from the formulation that did not contain any propylene glycol.

TABLE 18
A) Aqueous gel with 0% PG, batch 1121-41A

| Time | Cell | Dose (mg) |
|---|---|---|
| 0 hours | A1 | 9.0 |
| | A2 | 9.2 |
| | A3 | 9.6 |
| | D6 | 9.4 |
| | D7 | 8.8 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | A1 | 12.4497 | 6.0230 | 0.8 | | | |
| | A2 | 13.2937 | 6.0183 | 1.4 | | | |
| | A3 | 12.7629 | 6.0017 | 1.1 | | | |
| | D6 | 12.4133 | 6.0593 | 1.5 | | | |
| | D7 | 12.3162 | 6.0035 | 1.5 | 1.3 | 0.308 | 24.5% |
| 20 hours | A1 | 12.4702 | 6.0040 | 2.0 | | | |
| | A2 | 12.8687 | 6.0113 | 3.3 | | | |
| | A3 | 12.1943 | 6.0181 | 2.9 | | | |
| | D6 | 12.2564 | 6.0230 | 3.5 | | | |
| | D7 | 12.2177 | 6.0165 | 3.7 | 3.1 | 0.645 | 21.0% |
| 24 hours | A1 | 12.4009 | 6.0054 | 2.2 | | | |
| | A2 | 12.8173 | 6.0054 | 3.5 | | | |
| | A3 | 12.3596 | 6.0029 | 3.1 | | | |
| | D6 | 12.1645 | 6.0927 | 3.7 | | | |
| | D7 | 12.1785 | 6.0458 | 3.8 | 3.3 | 0.655 | 20.0% |

B) Aqueous gel with 5% PG, batch 1121-41B

| Time | Cell | Dose (mg) |
|---|---|---|
| 0 hours | A4 | 9.0 |
| | A5 | 8.5 |
| | A6 | 8.8 |
| | D4 | 8.6 |
| | D5 | 9.4 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | A4 | 12.7915 | 6.0045 | 0.9 | | | |
| | A5 | 12.7914 | 6.0046 | 1.1 | | | |
| | A6 | 12.6556 | 6.0261 | 0.6 | | | |
| | D4 | 12.8968 | 6.0224 | 0.7 | | | |
| | D5 | 13.2581 | 6.0113 | 0.5 | 0.7 | 0.230 | 30.9% |
| 20 hours | A4 | 11.9598 | 6.0256 | 3.0 | | | |
| | A5 | 12.5015 | 6.0008 | 3.3 | | | |
| | A6 | 12.6008 | 6.0230 | 2.1 | | | |
| | D4 | 12.6304 | 6.0062 | 3.0 | | | |
| | D5 | 12.6298 | 6.0102 | 1.8 | 2.6 | 0.639 | 24.1% |
| 24 hours | A4 | 11.9305 | 6.0061 | 3.2 | | | |
| | A5 | 12.5073 | 6.0222 | 3.5 | | | |
| | A6 | 12.6270 | 6.0191 | 2.2 | | | |
| | D4 | 12.6524 | 6.0318 | 3.2 | | | |
| | D5 | 12.5273 | 6.0399 | 2.0 | 2.8 | 0.667 | 23.8% |

C) Aqueous gel with 10% PG, batch 1121-41C

| Time | Cell | Dose (mg) |
|---|---|---|
| 0 hours | A7 | 9.3 |
| | A8 | 8.9 |
| | D1 | 9.1 |
| | D2 | 9.8 |
| | D3 | 9.0 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | A7 | 12.4646 | 6.0078 | 1.1 | | | |
| | A8 | 11.7522 | 6.0489 | 1.1 | | | |
| | D1 | 12.2496 | 6.0131 | 0.6 | | | |
| | D2 | 12.3044 | 6.0100 | 0.4 | | | |
| | D3 | 12.8053 | 6.0003 | 0.5 | 0.7 | 0.357 | 48.2% |
| 20 hours | A7 | 12.4183 | 6.0046 | 4.5 | | | |
| | A8 | 11.5798 | 6.0155 | 4.4 | | | |
| | D1 | 12.2057 | 6.0025 | 3.2 | | | |
| | D2 | 12.0376 | 6.0713 | 2.3 | | | |
| | D3 | 12.2851 | 6.0316 | 2.6 | 3.4 | 1.016 | 29.6% |
| 24 hours | A7 | 12.4588 | 6.0168 | 4.9 | | | |
| | A8 | 11.5998 | 6.0032 | 4.7 | | | |
| | D1 | 12.3128 | 6.0045 | 3.4 | | | |
| | D2 | 12.1818 | 6.0034 | 2.5 | | | |
| | D3 | 12.4816 | 6.0266 | 2.8 | 3.6 | 1.082 | 29.7% |

D) Aqueous gel with 15% PG, batch 1121-43A

| Time | Cell | Dose (mg) |
|---|---|---|
| 0 hours | B1 | 9.8 |
| | B2 | 9.1 |
| | B3 | 9.7 |
| | C4 | 9.5 |
| | C5 | 9.4 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | B1 | 12.3345 | 6.0145 | 0.4 | | | |
| | B2 | 12.5182 | 6.0332 | 0.7 | | | |
| | B3 | 12.8686 | 6.0069 | 0.4 | | | |
| | C4 | 12.9820 | 6.0105 | 0.6 | | | |
| | C5 | 12.2445 | 6.0264 | 0.3 | 0.5 | 0.149 | 30.9% |
| 20 hours | B1 | 12.2791 | 6.0052 | 2.9 | | | |
| | B2 | 12.2073 | 6.0384 | 3.7 | | | |
| | B3 | 12.6255 | 6.0136 | 2.7 | | | |
| | C4 | 12.7526 | 6.0094 | 3.8 | | | |
| | C5 | 12.0930 | 6.0164 | 2.9 | 3.2 | 0.519 | 16.1% |
| 24 hours | B1 | 12.3827 | 6.0230 | 3.2 | | | |
| | B2 | 12.3853 | 6.0339 | 4.0 | | | |
| | B3 | 12.6569 | 6.0398 | 2.9 | | | |
| | C4 | 12.6724 | 6.0121 | 4.0 | | | |
| | C5 | 12.3004 | 6.0317 | 3.1 | 3.4 | 0.521 | 15.1% |

E) Aqueous gel with 20% PG, batch 1121-43B

| Time | Cell | Dose (mg) |
|---|---|---|
| 0 hours | B4 | 9.9 |
| | B5 | 9.4 |
| | C1 | 8.8 |
| | C2 | 9.0 |
| | C3 | 9.0 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | A4 | 12.5430 | 6.0515 | 0.2 | | | |
| | A5 | 12.4326 | 6.0011 | 0.7 | | | |
| | A6 | 11.9923 | 6.0522 | 0.2 | | | |
| | D4 | 12.2172 | 6.0132 | 0.7 | | | |
| | D5 | 12.1329 | 6.0301 | 0.3 | 0.4 | 0.240 | 58.7% |
| 20 hours | A4 | 12.2239 | 6.0457 | 2.4 | | | |
| | A5 | 12.1429 | 6.0466 | 4.5 | | | |
| | A6 | 11.9431 | 6.0201 | 3.4 | | | |
| | D4 | 12.2475 | 6.0094 | 4.6 | | | |
| | D5 | 12.0741 | 6.0097 | 3.3 | 3.7 | 0.945 | 25.9% |
| 24 hours | A4 | 12.2894 | 6.0044 | 2.6 | | | |
| | A5 | 12.1950 | 6.0181 | 4.9 | | | |
| | A6 | 11.9515 | 6.0143 | 3.6 | | | |
| | D4 | 12.3029 | 6.0171 | 4.9 | | | |
| | D5 | 12.0401 | 6.0471 | 3.6 | 3.9 | 0.968 | 24.6% |

TABLE 19

| | | Single Tape- | | | | Dose |
|---|---|---|---|---|---|---|
| Formulation | | strip | Epidermis | Dermis | Receptor | Recovered |
| Aqueous Gel with | Mean | 26.17 | 48.18 | 0.47 | 3.27 | 87.52 |
| 0% PG batch | SD | 6.19 | 8.80 | 0.35 | 0.65 | 9.85 |
| 1121-41A | % CV | 23.65 | 18.26 | 74.46 | 20.01 | 11.26 |
| Aqueous Gel with | Mean | 10.06 | 15.38 | 0.15 | 2.80 | 82.63 |
| 5% PG batch 1121- | SD | 1.91 | 3.49 | 0.09 | 0.67 | 4.75 |
| 41B | % CV | 19.00 | 22.67 | 58.91 | 23.80 | 5.74 |
| Aqueous Gel with | Mean | 11.73 | 17.58 | 0.36 | 3.65 | 94.45 |
| 10% PG batch | SD | 2.61 | 7.11 | 0.21 | 1.08 | 8.80 |
| 1121-41C | % CV | 22.22 | 40.42 | 58.29 | 29.68 | 9.32 |
| Aqueous Gel with | Mean | 10.62 | 22.21¹ | 0.38 | 3.44 | 93.16 |
| 15% PG batch | SD | 3.04 | 8.12 | 0.13 | 0.52 | 3.84 |
| 1121-43A | % CV | 28.63 | 36.55 | 33.44 | 15.11 | 4.12 |
| Aqueous Gel with | Mean | 11.53 | 22.45¹ | 0.22 | 3.94 | 99.83 |
| 20% PG batch | SD | 3.22 | 6.79 | 0.09 | 0.97 | 6.73 |
| 1121-43B | % CV | 27.89 | 30.26 | 42.22 | 24.58 | 6.74 |

Note:
Values are in % of Applied Dose;
N = 5 cells per formulation except where noted.
¹N = 4

Results from the formulations of XMP.629 listed in Table 15 showed that skin penetration of the four tested formulations ranged from 2.3 to 2.6 percent of the applied dose as presented in Table 20. Epidermal levels, which include most of the stratum corneum post tape-strip, ranged from 3.2% to 10.8% of the applied dose as shown in Table 21. The highest epidermal levels were observed with 0% propylene glycol at 10.8% of the applied dose. The highest dermal levels (0.60% of the applied dose) were obtained with 0% propylene glycol. Dermal levels ranged from 0.05% to 0.60% of the applied dose as shown in Table 21. Dose recovery ranged from 66% to 85%. Dose recovery was low and variable, presumably due to binding of the drug to the cotton swabs used to remove formulation from the skin surface at 24 hours. These results showed that (1) in vitro skin penetration of XMP.629 was not appreciably affected by varying the concentration of propylene glycol and HEC in the aqueous gel formulation; (2) a trend for increased drug epidermal deposition was observed in formulations with lower PG concentration, as noted in the significant difference between formulations A&F, A&G, B&F (p<0.05, unpaired t-test); (3) a trend for increased drug epidermal deposition was observed in formulations with lower HEC concentration, as noted in the significant difference between formulations A&B (p<0.05, unpaired t-test); and (4) a trend for increased drug dermal deposition was observed in formulations with lower PG concentration, as noted in the significant difference between formulations B&F, D&F (p<0.05, unpaired t-test).

TABLE 20
A) 1121-77A

| Time | Cell | Dose (mg) |
|---|---|---|
| 0 | A1 | 9.1 |
| | A2 | 8.7 |
| | A3 | 9.0 |
| | D1 | 9.1 |
| | D2 | 9.1 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | A1 | 12.2932 | 6.6782 | 2.866 | | | |
| | A2 | 12.4119 | 6.3006 | 2.176 | | | |
| | A3 | 12.1578 | 6.1435 | 1.248 | | | |
| | D1 | 12.3842 | 6.4696 | 1.811 | | | |
| | D2 | 12.3354 | 6.4046 | 1.262 | 1.87 | 0.68 | 36.3% |
| 20 hours | A1 | 12.2663 | 6.1903 | 3.378 | | | |
| | A2 | 12.3195 | 6.4654 | 2.861 | | | |
| | A3 | 12.3158 | 6.3412 | 1.484 | | | |
| | D1 | 12.3115 | 6.1202 | 2.175 | | | |
| | D2 | 12.1902 | 6.0965 | 1.538 | 2.29 | 0.83 | 36.2% |
| 24 hours | A1 | 12.3497 | 6.481 | 3.412 | | | |
| | A2 | 12.4584 | 6.3288 | 2.905 | | | |
| | A3 | 12.3698 | 6.2333 | 1.509 | | | |
| | D1 | 12.2587 | 6.0790 | 2.209 | | | |
| | D2 | 12.0600 | 6.1346 | 1.569 | 2.32 | 0.83 | 35.9% |

B) 1121-77B

| Time | Cell | Dose (mg) |
|---|---|---|
| 0 | A4 | 8.7 |
| | A5 | 8.7 |
| | D3 | 8.6 |
| | D4 | 8.7 |
| | D5 | 8.7 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | A4 | 12.6475 | 6.3146 | 1.674 | | | |
| | A5 | 12.5123 | 6.1765 | 2.734 | | | |
| | D3 | 12.0348 | 6.5058 | 2.467 | | | |
| | D4 | 12.9347 | 6.5908 | 1.803 | | | |
| | D5 | 12.7296 | 6.1016 | 1.602 | 2.056 | 0.511 | 24.9% |
| 20 hours | A4 | 12.6096 | 6.0023 | 2.172 | | | |
| | A5 | 12.5944 | 6.2768 | 3.279 | | | |
| | D3 | 12.1188 | 6.5990 | 3.153 | | | |
| | D4 | 12.9803 | 6.1791 | 2.341 | | | |
| | D5 | 12.7234 | 6.4932 | 2.019 | 2.593 | 0.582 | 22.4% |
| 24 hours | A4 | 12.6091 | 6.3925 | 2.208 | | | |
| | A5 | 12.6584 | 6.1567 | 3.327 | | | |
| | D3 | 12.0409 | 6.1140 | 3.218 | | | |
| | D4 | 13.0116 | 6.1529 | 2.405 | | | |
| | D5 | 12.7154 | 6.3663 | 2.076 | 2.647 | 0.584 | 22.1% |

C) 1121-77C

| Time | Cell | Dose (mg) |
|---|---|---|
| 0 | A6 | 8.7 |
| | A7 | 8.6 |
| | B1 | 8.4 |
| | D6 | 8.8 |
| | D7 | 8.7 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | A6 | 12.7825 | 6.0980 | 2.851 | | | |
| | A7 | 12.5478 | 6.0576 | 1.810 | | | |
| | B1 | 12.3957 | 6.2059 | 1.863 | | | |
| | D6 | 12.5675 | 6.2940 | 2.891 | | | |
| | D7 | 12.8316 | 6.5896 | 0.850 | 2.053 | 0.849 | 41.3% |
| 20 hours | A6 | 12.8035 | 6.2441 | 3.545 | | | |
| | A7 | 12.5225 | 6.1721 | 2.392 | | | |
| | B1 | 12.2662 | 6.6601 | 2.417 | | | |
| | D6 | 12.4839 | 6.2691 | 3.405 | | | |
| | D7 | 12.5530 | 6.6132 | 1.142 | 2.580 | 0.967 | 37.5% |
| 24 hours | A6 | 12.8014 | 6.5243 | 3.601 | | | |
| | A7 | 12.4267 | 6.3302 | 2.451 | | | |
| | B1 | 12.2599 | 6.1846 | 2.463 | | | |
| | D6 | 12.5453 | 6.0802 | 3.438 | | | |
| | D7 | 12.5682 | 6.2852 | 1.180 | 2.627 | 0.969 | 36.9% |

D) 1121-77D

| Time | Cell | Dose (mg) |
|---|---|---|
| 0 | A8 | 8.7 |
| | A9 | 8.6 |
| | B2 | 8.9 |
| | C1 | 9.0 |
| | C2 | 9.0 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | A8 | 12.2803 | 6.3070 | 2.514 | | | |
| | A9 | 12.5699 | 6.4370 | 1.082 | | | |
| | B2 | 12.6741 | 6.2270 | 2.738 | | | |
| | C1 | 12.4459 | 6.0239 | 1.598 | | | |
| | C2 | 12.4418 | 6.2404 | 0.741 | 1.735 | 0.873 | 50.3% |
| 20 hours | A8 | 12.22074 | 6.3666 | 3.238 | | | |
| | A9 | 12.4008 | 6.5550 | 1.423 | | | |
| | B2 | 12.4646 | 6.1188 | 3.233 | | | |
| | C1 | 12.2271 | 6.2423 | 1.971 | | | |
| | C2 | 12.3806 | 3.3210 | 1.368 | 2.247 | 0.933 | 41.5% |
| 24 hours | A8 | 12.6311 | 6.1152 | 3.315 | | | |
| | A9 | 12.5124 | 6.3092 | 1.466 | | | |
| | B2 | 12.2805 | 6.3457 | 3.278 | | | |
| | C1 | 12.3247 | 6.0881 | 2.014 | | | |
| | C2 | 12.3890 | 6.1019 | 1.413 | 2.297 | 0.942 | 41.0% |

E) 1121-77^{E}

| Time | Cell | Dose (mg) |
|---|---|---|
| 0 | B3 | 8.9 |
| | B4 | 8.9 |
| | C3 | 9.1 |
| | C4 | 8.6 |
| | C5 | 8.8 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | B3 | 12.7283 | 6.1345 | 3.028 | | | |
| | B4 | 12.9388 | 6.0793 | 1.508 | | | |
| | C3 | 12.2463 | 6.3710 | 2.661 | | | |
| | C4 | 12.5525 | 6.2322 | 1.691 | | | |
| | C5 | 12.8005 | 6.3205 | 1.402 | 2.058 | 0.737 | 35.8% |
| 20 hours | B3 | 12.5144 | 6.4498 | 3.498 | | | |
| | B4 | 12.4257 | 6.6717 | 1.861 | | | |
| | C3 | 12.0239 | 6.7160 | 3.186 | | | |
| | C4 | 12.3822 | 6.0429 | 2.238 | | | |
| | C5 | 12.6384 | 6.0845 | 1.894 | 2.535 | 0.759 | 29.9% |
| 24 hours | B3 | 12.6509 | 6.2693 | 3.530 | | | |
| | B4 | 12.5229 | 6.3603 | 1.899 | | | |
| | C3 | 12.0841 | 6.5084 | 3.234 | | | |
| | C4 | 12.4603 | 6.3580 | 2.308 | | | |
| | C5 | 12.4880 | 6.3030 | 1.957 | 2.586 | 0.751 | 29.0% |

F) 1121-77F

| Time | Cell | Dose (mg) |
|---|---|---|
| 0 | B5 | 8.8 |
| | B6 | 8.7 |
| | C6 | 8.7 |
| | C7 | 8.6 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | B5 | 12.5224 | 6.4379 | 2.638 | | | |
| | B6 | 12.6440 | 6.1683 | 3.332 | | | |
| | C6 | 11.6652 | 6.6957 | 0.978 | | | |
| | C7 | 12.4093 | 6.0447 | 0.910 | 1.964 | 1.212 | 61.7% |
| 20 hours | B5 | 12.2031 | 6.0852 | 3.069 | | | |
| | B6 | 12.4213 | 6.6130 | 4.235 | | | |
| | C6 | 11.6966 | 6.5926 | 1.275 | | | |
| | C7 | 12.3428 | 6.2583 | 1.089 | 2.417 | 1.505 | 62.3% |
| 24 hours | B5 | 12.1230 | 6.2110 | 3.108 | | | |
| | B6 | 12.4306 | 6.4063 | 4.283 | | | |
| | C6 | 11.7774 | 6.2794 | 1.304 | | | |
| | C7 | 12.3668 | 6.2192 | 1.112 | 2.452 | 1.516 | 61.8% |

G) 1121-80

| Time | Cell | Dose (mg) |
|---|---|---|
| 0 | B7 | 9.1 |
| | B8 | 8.9 |
| | C8 | 8.5 |
| | C9 | 9.1 |

| | | Receptor | Receptor | | | | |
|---|---|---|---|---|---|---|---|
| | | Collected | Aliquot | Cumulative | Cumulative | | |
| Time | Cell | (g) | (g) | % dose | mean | stdev | % CV |
| 3 hours | B7 | 12.5370 | 6.3572 | 2.070 | | | |
| | B8 | 12.5389 | 6.2156 | 2.411 | | | |
| | C8 | 12.5297 | 6.5967 | 1.218 | | | |
| | C9 | 12.3137 | 6.1474 | 0.937 | 1.659 | 0.695 | 41.9% |
| 20 hours | B7 | 12.4848 | 6.4335 | 2.726 | | | |
| | B8 | 12.3621 | 6.6752 | 3.094 | | | |
| | C8 | 12.5250 | 6.6745 | 1.600 | | | |
| | C9 | 12.2242 | 6.0551 | 1.602 | 2.256 | 0.771 | 34.2% |
| 24 hours | B7 | 12.4809 | 6.2758 | 2.762 | | | |
| | B8 | 12.4348 | 6.3283 | 3.142 | | | |
| | C8 | 12.4566 | 6.3732 | 1.661 | | | |
| | C9 | 12.3136 | 6.3699 | 1.632 | 2.299 | 0.769 | 33.5% |

TABLE 21

| | | Single | | | | Dose |
|---|---|---|---|---|---|---|
| Formulation | | Tape-strip | Epidermis | Dermis | Receptor | Recovered |
| Aqueous Gel with 0% | Mean | 13.61 | 10.83 | 0.60 | 2.32 | 66.11 |
| PG and 1.25% HEC, | SD | 5.74 | 2.51 | 0.48 | 0.83 | 2.07 |
| batch 1121-77A | % CV | 42.17 | 23.17 | 79.09 | 35.86 | 3.12 |
| Aqueous Gel with 0% | Mean | 8.87 | 5.20 | 0.16 | 2.65 | 83.57 |
| PG and 1.5% HEC, | SD | 1.80 | 1.14 | 0.07 | 0.58 | 3.32 |
| batch 1121-77B | % CV | 20.29 | 21.91 | 46.75 | 22.07 | 3.97 |
| Aqueous Gel with 1% | Mean | 12.41 | 9.02 | 0.12 | 2.63 | 84.87 |
| PG and 1.25% HEC, | SD | 3.51 | 5.30 | 0.10 | 0.97 | 4.75 |
| batch 1121-77C | % CV | 28.32 | 58.82 | 83.89 | 36.91 | 5.60 |
| Aqueous Gel with 1% | Mean | 10.72 | 6.42 | 0.13 | 2.30 | 77.68 |
| PG and 1.5% HEC, | SD | 5.86 | 3.45 | 0.04 | 0.94 | 4.16 |
| batch 1121-77D | % CV | 54.67 | 53.68 | 33.41 | 41.01 | 5.36 |
| Aqueous Gel with 2% | Mean | 7.42 | 7.09 | 0.12 | 2.59 | 73.39 |
| PG and 1.25% HEC, | SD | 4.14 | 5.15 | 0.09 | 0.75 | 3.99 |
| batch 1121-77E | % CV | 55.78 | 72.64 | 70.56 | 29.04 | 5.44 |
| Aqueous Gel with 2% | Mean | 7.06 | 3.17 | 0.05 | 2.45 | 74.53 |
| PG and 1.5% HEC, | SD | 4.21 | 1.37 | 0.04 | 1.52 | 4.57 |
| batch 1121-77F | % CV | 59.64 | 43.36 | 73.57 | 61.83 | 6.13 |
| Aqueous Gel with 5% | Mean | 5.96 | 3.73 | 0.10 | 2.30 | 78.30 |
| PG and 1.5% HEC, | SD | 1.13 | 0.90 | 0.04 | 0.77 | 10.30 |
| batch 1121-80 | % CV | 19.01 | 24.07 | 41.77 | 33.46 | 13.15 |

Note:
Values are in % of Applied Dose;
N = 5 cells per formulation for A-E and N = 4 cells per formulation for F-G

Additional in vitro percutaneous absorption analyses were done to determine if XMP.629 translocated to the pilosebaceous unit (hair follicle and associated sebaceous glands and ducts) of the epithelium. Colonization of the pilosebaceous unit by a microbial consortium, including, for example, Propionibactedum acnes Staphylococcus epidermis may be involved in the pathogenesis of acne vulgaris.

The skin used in these analysis were human abdominal skin from a single donor who had undergone cosmetic elective surgery. This study evaluated the same formulations used in the in vitro percutaneous absorption study described above. These formulations were three gels (two containing ethanol) and one lotion as described above. The compositions of these formulations are summarized in Table 13 above.

Skin samples were mounted on Franz diffusion cells and 8.8 mg of each formulation (5 mg/cm) was spread onto the skin for a 24 hour exposure period. After the 24 hour exposure period the skin samples were wiped with cotton swabs and stored at −20° C. prior to being shipped and analyzed for immunostaining. Immunostaining was performed using a rabbit anti-XMP.629 polyclonal antibody. The polyclonal antibody was produced in rabbits and rabbit serum was used to affinity purify the antibody.

For immunostaining, the tissues were sectioned into 5 μM sections, fixed in acetone for 5 minutes and then dried overnight. They were then fixed in neutral buffered formalin for 10 minutes prior to staining. The actual immunostaining procedure was an indirect immunoperoxidase method. Acetone/formalin-fixed cryosections were rinsed twice in phosphate-buffered saline (PBS, [0.15M NaCl, pH 7.2]). Endogenous peroxidase was quenched by incubation of the slides with glucose oxidase (1 U/mL, Sigma, St. Louis, Mo.)/glucose (10 mM) and sodium azide (1 mM) for one hour at 35° C. The slides were then rinsed two times with PBS (0.15M NaCl, pH 7.2). Next, the slides were blocked with avidin solution (Avidin Biotin Blocking Kit, Vector Laboratories, Burlingame, Calif.) for 15 minutes, rinsed with PBS (0.15M NaCl, pH 7.2), followed by blocking with biotin solution (Avidin Biotin Blocking Kit, Vector Laboratories, Burlingame, Calif.) for 15 minutes at room temperature, and rinsed with PBS (0.15M NaCl, pH 7.2). This was followed by application of a protein block designed to reduce nonspecific binding. The protein block was prepared as follows: phosphate-buffered saline (PBS [0.15 M NaCl], pH 7.2); 0.5% casein; 1% BSA; and 1.5% normal horse serum. Following the protein block, the primary antibodies [rabbit polyclonal anti-XMP.629 or control rabbit IgG1 (RbIgG, Dako, Carpenteria, Calif.)] were applied to the slides and incubated for one hour at room temperature. Next, the slides were rinsed two times with PBS (0.15M NaCl, pH 7.2), and the biotinylated secondary antibody (Biotinylated donkey anti-RbIgG, Jackson lmmunoresearch, West Grove, Pa.) was applied for 30 minutes, rinsed two times with PBS (0.15M NaCl, pH 7.2), and treated with the ABC Elite reagent (ABC “Elite” Kit, Vector Laboratories, Burlingame, Calif.) for 30 minutes. The slides were then rinsed two times with PBS (0.15M NaCl, pH 7.2) and treated with 3,3′-diaminobenzidine tetrahydrochloride (DAB, Sigma, St. Louis, Mo.) for 4 minutes. All slides were counterstained with hematoxylin, dehydrated and coverslipped for interpretation.

PBS (0.15 M NaCl, pH 7.2) with 1% BSA served as the diluent for all antibodies. PBS (0.15M NaCl, pH 7.2) was used in all rinse steps. All slides were read by the Study Pathologist to identify the tissue or cell type stained and intensity of staining (graded+[equivocal], 1+[weak], 2+[moderate], 3+[strong], 4+[intense]Neg [negative]). All slides were judged for adequacy of tissue elements and staining.

The results of the immunostaining procedure are summarized in Table 22 and Table 23. Table 22 summarizes these data according to the intensity of staining and Table 23 summarizes these data by the number of cells in each section which stained positive. The last column in Table 22 entitled “Follicle Total” is the summation of the pathologist-reported values contained in the “Hair Follicle” column minus the vehicle control values. When examining the data contained in Table 22 it is important to keep in mind that these summations were performed by the study representative in an attempt to add a semi-quantitative aspect to the results so as to ease comparison between the different formulations. In the same vein, the data in the column “Total Follicle Fictional Numerical Value” of Table 23 represent the summation of numerical values which have been assigned to the pathologist's written description of the number of cells which were stained in each sample. These summations should not be considered a rigorous, empirical measurement of staining, rather, both the summations should be taken as a semi-quantitative attempt to assign a numerical value to the staining with each formulation so as to allow ease of comparison between different formulations.

All of the drug-containing formulations delivered detectable amounts of XMP.629 into the hair follicle. The summations contained in Table 22 and Table 23 indicate that the staining associated with formulations 1121-18C and 1121-22A appeared to be stronger both in intensity of staining and in number of cells stained. The hair follicle summation values with formulation 1121-18C were slightly higher with 1121-18C, indicating increased XMP.629 localization with this formulation than with 1121-22A.

TABLE 22

| Formulation | Cells | Surface | Hair follicle | Follicle |
|---|---|---|---|---|
| 1121-18C | A1, A2, D3, | 2, 1, 1-2, 1 | 2, 0, 1, 1-2 | 4.5 |
| | D4 | | | |
| 1121-25A | A3, A4, D1, | 2, 0, 0, 1-2 | 2, 0, 0, 0 | 2.0 |
| | D2 | | | |
| 1121-25B | B1, B2, C3, | 0, 0, 1-2, 2 | 0, 0, 1-2, 2 | 1.5 |
| | C4 | | | |
| 1121-22A | B3, B4, C1, | 2, 2, 2-3, 2-3 | 2, 2, 0, 2-3 | 5.0 |
| | C2 | | | |

| VEHICLE | CELLS | | | |
|---|---|---|---|---|
| 1121-30A (1121-18C | A5 | 0 | 0 | 0 |
| control) | | | | |
| 1121-32A (1121-25A | B5 | 0 | 0 | 0 |
| control) | | | | |
| 1121-32B (1121-25B | C5 | 0 | 2 | 2 |
| control) | | | | |
| 1121-33A (1121-22A | D5 | 1-2 | 1-2 | 1.5 |
| control) | | | | |

*A summation of the values from the Hair Follicle column with vehicle control values subtracted. In performing the summation the values 1-2 were taken to equal 1.5 and the values 2-3 were taken to equal 2.5

TABLE 23

| Formu- | | | |
|---|---|---|---|
| lation | Cells | Surface | Hair follicle |
| 1121-18C | A1, A2, D3, D4 | Occasional, | Occasional, |
| | | Very Rare | Negative, |
| | | Occasional, Rare | Rare, Occasional |
| 1121-25A | A3, A4, D1, D2 | Rare, Negative, | Rare, Negative, |
| | | Negative, Rare | Negative, Negative |
| 1121-25B | B1, B2, C3, C4 | Negative, Negative, | Negative, Negative, |
| | | Occasional, Rare | Occasional, Rare |
| 1121-22A | B3, B4, C1, C2 | Occasional, | Rare, Occasional, |
| | | Occasional, | Occasional to |
| | | Occasional to | Frequent, Negative |
| | | Frequent, | |
| | | Occasional | |

| VEHICLE | CELLS | | |
|---|---|---|---|
| 1121-30A (1121-18C | A5 | Negative | Negative |
| control) | | | |
| 1121-32A (1121-25A | B5 | Negative | Negative |
| control) | | | |
| 1121-32B (1121-25B | C5 | Negative | Occasional |
| control) | | | |
| 1121-33A (1121-22A | D5 | Rare to | Rare to Occasional |
| control) | | Occasional | |

*A summation of the values from the Hair Follicle column with vehicle control values subtracted.
0 = Neg.;
1 = Very Rare;
2 = Rare;
2.5 = Rare to Occasional;
3 = Occasional;
3.5 = Occasional to Frequent.

### EXAMPLE 7

### Toxicity Studies

This example addresses toxicity studies, including mutagenicity, irritation, and parental studies, conducted with XMP.629.

A. Genotoxicity/Mutagenicity Studies

Three representative tests, Ames, mouse micronucleus, and Chinese Hamster Ovary (CHO) chromosomal aberration, were performed to assess the mutagenicity of XMP.629.

A1. Ames Test

XMP.629 was evaluated in an initial mutagenicity assay. In this Salmonella-Escherchia coli/mammalian-microsome reverse mutation assay, XMP.629 was evaluated for the ability to induce reverse mutations at the histidine locus in four tester strains of Salmonella-typhimurium (TA98, TA100, TA1535 and TA1537) and at the tryptophan locus in Escherichia coli tester strain, WP2ᵤᵥᵣA, in the presence or absence of an exogenous metabolic activation system (59). The experimental materials, methods and procedures were based on those described by Ames et al., 1975 (Mutat. Res. 31; 347-364) and Green and Muriel, 1976 (Mutat. Res. 38: 3-32). The assay design was based on the OECD Guideline 471, updated and adopted Jul. 21, 1997. Based on the results of an initial dose rangefinding study, XMP.629 was evaluated using (a) four standard Salmonella tester strains with the rat hepatic S9 (microsomal) fraction at doses of 1.00, 3.33, 10.0, 33.3, 66.7 and 100 μg/plate, and without S9 at doses of 1.00, 3.33, 10.0, 25.0, 33.3 and 66.7 μg/plate; and (b) tester strain WP2uvrA with S9 at doses of 3.33, 10.0, 33.3, 100, 250 and 333 μg/plate and without S9 at doses of 1.00, 3.33, 10.0, 33.3, 66.7 and 100 μg/plate. XMP.629 was re-evaluated in an independent confirmatory experiment under similar conditions at lower doses.

Inhibited growth was observed in tester strains TA100 and WP2uvrA at the highest two doses with S9, and in all five tester strains at the highest 1 to 3 doses without S9. Thinning of the background lawns (unaccompanied by a decrease in revertant frequencies) also was observed in the other three tester strains at the highest two doses with S9. In addition, the test article was freely soluble at all doses evaluated with and without S9. Revertant frequencies for all doses of XMP.629 tested in all five tester strains with and without S9 approximated were less than those observed in the concurrent vehicle controls. Therefore, XMP.629 was determined to be negative in the Salmonella-Escherichia coli/mammalian-microsome reverse mutation assay.

A2. Mouse Micronucleus Test

XMP.629 was next evaluated for in vivo clastogenic activity and/or disruption of mitotic apparatus by detecting micronuclei in polychromatic erythrocyte (PCE) cells in Crl:CD-1®(ICR) BR mouse bone marrow. The assay design was based on OECD Guideline 474, updated and adopted Jul. 21, 1997. In this micronucleus assay, XMP.629 was formulated in 10 mM sodium acetate buffer (pH 6.0) and dosed by intraperitoneal injection to six males per dose level at each scheduled harvest timepoint. The dose levels were 2, 4, or 8 mg/kg. Five animals dosed with XMP.629 at 2 or 4 mg/kg and five animals dosed with a positive control were euthanized approximately 24 hours after dosing for extraction of the bone marrow. Five animals per harvest timepoint dosed with XMP.629 at 8 mg/kg and five animals per harvest timepoint dosed with the vehicle control article were euthanized approximately 24 or 48 hours after dosing for extraction of the bone marrow. This micronucleus test can serve as a rapid screen for compounds which interfere with normal mitotic cell division (Schmid, 1975, Mutat. Res. 31: 9-15; Heddle et al., 1983, Mutat. Res. 123: 61-118; Heddle et al., 1991, Env. And Mol. Mutagen. 18: 277-291). At least 2000 PCEs per animal were analyzed for the frequency of micronuclei. Cytotoxicity was assessed by scoring the number of PCEs and normochromatic erythrocytes (NCEs) in at least the first 500 erythrocytes for each avenal. XMP.629 did not induce any signs of clinical toxicity in any of the treated animals at up to 8 mg/kg. XMP.629 did not induce any statistically significant increases in micronucleated PCEs at any of the doses examined. In addition, XMP.629 was not cytotoxic to the bone marrow, as there were no statistically significant decreases in the PCE:NCE ratios observed at any dose level tested. Therefore, XMP.629 was determined to be negative in the mouse bone marrow micronucleus assay.

A3. CHO Chromosomal Aberration Test

XMP.629 was next evaluated for activity to induce chromosomal aberrations in cultured CHO cells with and without an exogenous metabolic activation system. Aberrations are a consequence of failure or mistakes in repair processes such that breaks either do not rejoin or rejoin in abnormal configurations (Evans, 1962, Intl. Rev. Cytol. 13: 221-321; Evans, 1976, pp. 1-29 in: Chemical Mutagens, Principles and Methods for their Detection, Vol. 4, Hollaender, A(ed.), Plenum Press, New York). The assay design for these chromosomal aberration studies was based on OECD Guideline 473, updated and adopted Jul. 21, 1997. XMP.629 was provided as a 20.0 mg/mL stock in 10 mM sodium acetate buffer (pH 6). This solution and its dilutions prepared in cell culture grade water were dosed at 10% vol/vol (100 pUL/mL). The vehicle control cultures were treated with 100 μL/mL of cell culture grade water. In the initial assay, the treatment period was for 3.0 hours with and without metabolic activation at various concentrations of XMP.629 (30, 60, 120, 240, 480, 686, 980, 1400 and 2000 μg/mL) and cultures were harvested 20.0 hours from the initiation of treatment. Those cultures that were treated with 30, 60 and 120 pg/mL were analyzed for chromosomal aberrations. No significant increase in cells with chromosomal aberrations, polyploidy or endoreduplication was observed. In a confirmatory assay that included lower doses, the treatment period was 20 hours without metabolic activation (3.75, 7.5,15, 30, 60, 90, 120, 160, 200 and 240 μg/mL) and 3 hours with metabolic activation (15, 30, 60, 90, 120, 160, 200, 240, 300 and 360 μg/mL).

Again, no significant increases in cells with chromosomal aberrations, polyploidy, or endoreduplication was observed. Therefore, XMP.629 was considered negative for inducing chromosomal aberrations in CHO cells with and without metabolic activation.

B. Acute and Subchronic Toxicity Studies

B1. Rabbit Primary Skin Irritation Test

XMP.629 was evaluated for any potential dermal irritant effects in the rabbit primary skin irritation test for this dermal irritation study, XMP.629 was tested on intact and abraded skin in three New Zealand White rabbits. All animals received 0.5 mL of XMP.629 on each intact and abraded dorsal skin application site. This was accomplished by placing a gauze patch directly on the skin of each application site and saturating the patch with 0.5 mL of the XMP.629 solution. The application sites were then wrapped with gauze bandaging and non-irritating semi-occlusive tape. Elizabethan collars were then applied for the 4 hour exposure period. At the end of the 4 hour exposure period, the collars and wrappings were removed and any residual XMP.629 removed. The test sites were evaluated using the standard Draize scoring system (Draize et al., 1944, J. Pharmacol, Exp. Ther. 82: 377-390) for erythema and edema within 30 to 60 minutes and 24, 48, and 72 hours following patch removal. The results showed that XMP.629 was only mildly irritating to the abraded skin and was not irritating to the intact skin of the rabbits tested.

B2. Rabbit Primary Eye Irritation Test

XMP.629 was evaluated in a rabbit primary eye irritation test for potential ocular irritant and/or corrosive effects when topically applied to the eye of three New Zealand White rabbits.

All animals received 0.1 mL of a 2% solution XMP.629 in 10 mM sodium acetate instilled into the right conjunctival sac and left in place for 24 hours. The control eye was untreated. The treated eye was washed out with 100 mL of lukewarm water for one to two minutes at approximately 24 hours post-dose. The treated and control eyes were examined at approximately 1, 24, 48, and 72 hours post-dose and on days 5, 8, and 15 for ocular irritation using the standard Draize scoring system (Draize et al., 1944, supra). Sodium fluorescein and ultraviolet light was used to examine for possible corneal injury.

Results from this test showed no effects on corneal opacity observed at any time point. For the iris, average scores of less than 1 were recorded at 48 hours post-instillation for the three rabbits evaluated, with only one rabbit having a score of 1 by 72 hours, and scores of 0 at days 5, 8 and 15. For the conjunctivae, scores of 1 and 2 were recorded at one hour and persisted throughout the 72 hour observation period for all three rabbits and on Days 5 and 8 for one rabbit. Similar scores of 1 and 2 were also observed for conjunctional chemosis, with slight to obvious swelling seen up to 48 hours for all three rabbits and slight swelling on Days 5 and 8 in two rabbits. These effects were mild and reversible with removal of solution from the eyes. Sodium fluorescein examinations revealed transient effects of 2% and 5% area affected at 24 hours post-instillation in two rabbits, but 20% area affected in one rabbit cornea. These effects were not present when examined again at 48 hours. These transient effects may have been attributable to the hypertonicity of the test article solution however, the 10 mm sodium acetate vehicle solution was not separately evaluated in this study.

Based on the overall results, 2% XMP.629 in 10 mM sodium acetate solution was determined to be mildly irritating, but the mild irritation is completely reversible, when administered to the conjunctival sac of rabbit eyes.

B3. Guinea Pig Sensitization (Maximization) Test

A sensitization (maximization) study was performed to evaluate the potential of XMP.629 to elicit skin sensitization reactions, such as allergic contact dermatitis, in guinea pigs via intradermal injection and topical patch applications. Procedures for conducting dermal studies that determine the potential of substances to induce delayed contact hypersensitivity are well-known and are described, for example, in Magnusson, B. et al., Allergic Contact Dermatitis in the Guinea Pig, Charles C. Thomas Publishing, Springfield, Ill., 1970 and Klecak, G., “Test Methods for Allergic Contact Dermatitis in Animals”, Dermatoxicology, 5^{th} Edition, Taylor & Francis Publishing, Washington D.C., pages 437-459, the disclosures of which are incorporated herein by reference. A total of forty two-month old guinea pigs, twenty male and twenty female, from the strain CrI:(HA)BR(Albino Hartley) were employed for the entire regimen of the sensitization study.

First, a preliminary range-finding test was conducted to determine the test concentration of XMP.629 to be used in the second test, induction phase intradermal injection and topical patch application tests. The range-finding test was performed (i) intradermally on two guinea pigs (one male and one female) by injecting concentrations of 2, 4, 8, 16, and 20 mg/mL XMP.629; and (ii) topically on three guinea pigs (two males and one female) by administering patches containing concentrations of 5, 10, 15, and 20 mg/mL XMP.629. Intradermal injections (two series of 5 injections of 0.1 mL in each injection) were administered on Day 1 to the shoulder/trunk area and the injected guinea pigs were observed at approximately 24 and 48 hours afterwards. Topical patches were prepared by spreading a thick even layer of 0.4 mL of XMP.629 over a 2×2 cm of Whatman No.3 filter paper. Topical patches were applied to the torso of the guinea pig and covered by an overlapping plastic adhesive tape followed by additional wrapping tape. After a 24 hour exposure period, the patches were removed and the application sites were washed to removed any excess XMP.629 that may be present. Application sites were observed at approximately 24 and 48 hours after topical patch administration. Intradermal injection sites and topical application sites were evaluated using a sensitization grade rating. Skin at the site of treatment was compared with the surrounding skin for signs of dermal irritation. Each site could attain a maximum possible score of 3, as scored according to the rating listed below:
0=No reaction
1=Scattered mild erythema
2=Moderate and diffuse erythema
3=Intense erythema and edema

Results from the preliminary range-finding test suggested the use of a concentration of 20 mg/mL XMP.629 for both the intradermal injection and the topical patch application test in the second induction phase test.

The second induction phase test was conducted as a two-stage operation with intradermal injections initially being administered followed one week later by exposure to a closed patch. For the intradermal injections, two series of three injections (total volume per injection was 0.1 mL; total concentration of XMP.629 was 20 mg/mL) were administered deep into the dermis located at the head to shoulder area in test guinea pigs. Following a period of 24 and 48 hours, evaluations of the injection area were recorded. One week (day 8) after the injections were completed, topical patches (total concentration of XMP.629 was 20 mg/mL) were applied to the torso of the guinea pigs and left in place for approximately 48 hours. Results from the induction phase tests showed that 4 mg/mL of XMP.629 was the highest non-irritating dose. Dermal irritation scores of one or higher were recorded for doses of 8 mg/mL of XMP.629.

A third, challenge (maximization) test was performed two weeks (day 22) after initiation of the second, induction phase tests. Both the guinea pigs that received intradermal injections or the topical patches from the second, induction phase test were administered topical patches of XMP.629 at a concentration of 4 mg/mL on the left and right flanks in the challenge phase. Patches were sealed to the flanks for approximately 24 hours before observations were recorded. Results from the challenge phase showed that animals administered with 4 mg/mL of XMP.629 did not score higher than one, indicating the presence of only scattered mild erythema. Most individual animals showed a zero score, especially at the 48 hour evaluation after the application of patches. Therefore, XMP.629 in a 10 mM of sodium acetate solution demonstrated a weak skin sensitizing response following topical patch challenge when induction dosing was given by intradermal injection and topical application.

B4. Rat Acute Oral Toxicity Test

XMP.629 was evaluated for acute toxicity following a single dose by oral gavage. The study consisted of three treatment groups of five male and five female CD [CrI:CD (SD) IGS BR] rats which received XMP.629 as a gel at concentrations of 0.1, 0.5, and 1.0% of XMP.629 respectively at a volume of 10 mL/kg. An additional group of five rats per sex served as the control and received control gel at the same volume. All rats were observed twice daily for morbidity, mortality, injury, and availability of food and water. Body weights were measured pretest and at different intervals during the study for all rats; observations for clinical signs were conducted pretest and at approximately 2 and 4 hours following the dose; and daily clinical observations were conducted daily until Day 14. At study termination, gross necropsies were conducted and macroscopic observations were recorded for all animals.

No treatment-related effects were observed on survival, body weights, or macroscopic evaluations. No clinical signs of systemic toxicity were noted at concentrations of 0.1 or 0.5% XMP.629. Decreased activity was observed at four hours after the administration of XMP.629 (1.0% XMP.629), in four males and one female, but this finding was resolved by Day 2.

Thus, transient, test article-related clinical observations that quickly resolved were observed following single oral doses of the XMP.629. Based on the data from this study, the minimum lethal dose was considered to be greater than the high dose of 1% XMP.629 (10 mg/mL).

B5. Rat Subchronic Toxicity Test

XMP.629 was evaluated for potential subchronic dose toxicity after at least 30 consecutive days of administration to CD®[CrI: CD® (SD)IGS BR] rats. This study consisted of four main study groups, each containing ten rats/sex/group, and four toxicokinetic (TK) groups each containing six rats/sex/group. Three of the four main study and TK groups received XMP.629 by bolus subcutaneous injection at dose levels of 0.3, 1.0, and 3.0 mg/kg/day and at a dose volume of 3.0 mL/kg. One main study and TK group each served as control groups and received a vehicle (saline/sodium acetate buffer, pH 6.0) at the same dose volume. The TK groups were used for the assessment of plasma XMP.629 concentrations and to evaluate potential effects from repeat-dose administration of XMP.629 on antibody formation in a standardized assay. Blood was collected for plasma analysis on Days 1 and 28. On Day 25, the TK groups were immunized with keyhole limpet hemocyanin (KLH) antigen by subcutaneous injection. On Day 31, blood was collected by cardiac puncture after carbon dioxide inhalation from all surviving rats in all TK groups, including controls for serum analysis to evaluate anti-KLH IgM antibody levels. After the final blood collection on Day 31, the surviving TK rats were discarded without further examination.

All rats were observed twice daily for morbidity, mortality, injury, and availability of food and water. Body weights were measured pretest and weekly for all rats and observations for clinical signs and measurements for food consumption were conducted weekly during the course of the study on main study rats. A functional observational battery (FOB) was conducted prior to exposure to test article and during Week 4 on main study rats. Ophthalmoscopic examinations were conducted pretest on all rats and prior to study termination on the main study rats. At the end of the treatment period for the main study rats, various hematology, clinical chemistry, and urinalysis parameters were evaluated. Urine was also collected and preserved for possible analysis of test article concentration. At study termination, complete necropsy examinations were performed for the main study rats, organ weights were taken, and selected tissues were microscopically examined.

There were no test article-related effects on clinical signs, survival, FOB assessments, body weights, food consumption, ophthalmoscopic evaluations, or on the immune system. Hematology changes, including increases in neutrophils and monocytes in both sexes and mild increases in percent reticulocytes in the females at 1.0 and 3.0 mg/kg/day, suggested that a mild inflammatory response was occurring. Test article-related macroscopic findings were noted in the injection sites of the treated animals across all dose levels, including red discoloration (males at 0.3 mg/kg/day and higher, and in one female at 1.0 mg/kg/day) and thickening at the injection sites (males and females at 1.0 and 3.0 mg/kg/day). Increases in organ weights occurred in the spleen of males (1.0 and 3.0 mg/kg/day) and females (all doses), liver of females (all doses), and adrenal glands of males (3.0 mg/kg/day). Microscopically, there were test article-related findings observed in the injection sites of most males and females across all dose levels and in the spleens of females and adrenal glands of males at 1.0 and 3.0 mg/kg/day. Other microscopic findings were either background or incidental lesions in rats and were considered unrelated to test article administration.

The main finding observed was injection site lesions in rats treated by subcutaneous injection of XMP.629 at doses of 0.3, 1.0, and 3.0 mg/kg/day for 30 days. A dose-related increase in the incidence and severity of both acute and chronic injection site inflammation was reported. These effects were noted macroscopically and microscopically. In addition, changes noted in other parameters (i.e., hematology and increased spleen and adrenal gland weights) were determined to be associated with the injection site inflammation. No evidence of systemic toxicity was observed at any dose level. Based on these data, the no-observed-effect level (NOEL) for systemic effects was greater than 3.0 mg/kg/day of XMP.629. A NOEL could not be determined for local effects.

B6. Minipig Subchronic Toxicity Test

XMP.629 was evaluated for potential toxicity when administered by a single daily dermal application to Hanford minipigs over the course of one month. The study group design and dosage levels tested were as follows:

| | | | | | | | Formulation |
|---|---|---|---|---|---|---|---|
| | | | | Multiple of | | | Dose |
| | No. of Animals | | | Human/Animal | Active Dosage Level^{a,b} | | Volume |
| Group | Male | Female | Material | Dose μg/kg/day | μg/kg/day | μg/m²/day | (mL/kg/day) |
| 1 | 5 | 5 | XMP.629 acetate | 0 | 0 | 0 | 0.07 |
| | | | gel, Placebo | | | | |
| 2 | 5 | 5 | XMP.629 acetate | 1 | 70 | 1680 | 0.07 |
| | | | gel, 0.1% | | | | |
| 3 | 5 | 5 | XMP.629 acetate | 5 | 350 | 8400 | 0.07 |
| | | | gel, 0.5% | | | | |
| 4 | 5 | 5 | XMP.629 acetate | 10 | 700 | 16,800 | 0.07 |
| | | | gel, 1.0% | | | | |

^{a}The test article formulation and vehicle will be applied once daily (q.d).
^{b}Estimated from the conversion multiple of 24 for a 10 kg animal (Freireich et al., 1966, Cancer Chemotherapy Reports 50 (5): 219-243

The test article or placebo control was administered once daily to the dorsal surface of each animal for 29 consecutive days. General health, mortality and moribundity checks were conducted twice daily. General health examinations were performed by a veterinarian once prior to in-life initiation and weekly during the study. The animals were examined daily for overt toxic signs (post-dose observations) between one and two hours following dosing. Detailed clinical observations and individual body weights were performed weekly, beginning on study day 0, and on the day of scheduled euthanasia. Dermal scoring was performed once per week and on the day of scheduled euthanasia.

Hematology, coagulation and clinical chemistry parameters were evaluated once prior to in-life initiation (day -9) and near the conclusion of the dosing phase (day 26). Blood samples were also collected for anti-test article analyses on study day 26 and toxicokinetic (TK) analyses on study days 0 and 27. Ophthalmological examinations were performed once prior to in-life initiation and just prior to the end of the dosing phase. All animals were subjected to a complete gross necropsy at the end of the dosing phase (days 29 and 30). Fresh organ weights were obtained for surviving animals and selected tissues were preserved from all pigs. All tissues collected at necropsy from all animals were examined microscopically. In addition, an approximate 5 gram liver sample was collected from all animals, perfused with iced saline, wrapped in foil, labeled, flushed with argon, immediately frozen with liquid nitrogen and stored at approximately −70° C. for future analysis.

The death of one control group male was associated with the blood collection procedure on day 26. All other animals survived to scheduled euthanasia following the treatment period. No clinical signs of toxicity were observed. Dermal observations were limited to a single incidence of grade 2 (well defined) erythema in one 70 μg/kg/day female. There were no toxicologically meaningful differences noted between the control and test article-treated groups in mean body weight, mean body weight, change, ophthalmology, hematology, coagulation, clinical chemistry or organ weight data.

Gross necropsy observations for the control male that died following the blood collection procedure included distended stomach and duodenum, stomach mucosa reddened, petechial hemorrhages of the heart, lung bullae and hemorrhagic tissues (thyroids and tissues surrounding the thyroids) due to antemortem procedures. There were no other remarkable gross necropsy findings noted in any animals at scheduled euthanasia. Microscopic examination of the collected tissues revealed no treatment-related lesions.

Analysis of serum samples obtained for toxicokinetic analysis revealed that all serum concentrations were below the limit of detection of the assay (1 ng/mL). No toxicokinetic analysis was performed.

Based on the results of this study, a dosage level of 700 pg/kg/day was determined to be a no-observed-effect level (NOEL) following dermal application of XMP.629 for 30 days.

B7. Mouse Dermal Carciogenicity Study

XMP.629 was evaluated in a dermal carcinogenicity study to determine whether repeated dermal treatment with tetradeconyl phorbol acetate (TPA) in a XMP.629 acetate gel placebo formulation increased the incidence of skin tumors in hemizygous Tg.AC mice. Four groups of five Tg.AC transgenic mice/sex (Groups 3-6) were treated with tetradeconyl phorbol acetate (TPA) in a XMP.629 acetate gel placebo at dose levels of 2.5, 5, 10 and 20 μg TPA per 150 μL application/mouse, respectively. Group 1 was treated with XMP.629 acetate gel placebo alone. A sixth group (Group 2) was treated with TPA dissolved in acetone at a dose level of 1.25 μg per 150 μL application and served as a reference standard. The animals were dosed via dermal application three times per week (e.g., Monday, Wednesday and Friday) for 12 consecutive weeks.

All animals were observed twice daily for moribundity and mortality. A hands-on examination was performed weekly at the time the animals were weighed on Day 1 and weekly thereafter. At that time, the animals were examined for clinical evidence of toxicity, carcinogenicity, and/or irritation at the site of application. The animals were observed at the site of application for development of latent or actual tumors once prior to dosing and weekly thereafter. The number of tumors at the site of application (SOA) and non-site of application (non-SOA) was recorded for each animal each week. At the end of the study (Day 85), the surviving animals were euthanized with CO2 asphyxiation. A necropsy was not performed.

Mortality was limited to one Group 1 female found dead on Day 76 and one Group 6 female found dead on Day 28. Treatment with TPA in XMP.629 acetate gel did not result in dermal irritation or have an effect on body weights.

Treatment with TPA in XMP.629 acetate gel placebo at 2.5 and 5.0 pg per application did not result in tumor development in any animal of either sex. A single female treated with 10 μg TPA per application developed one latent and one actual papilloma at the site of application. Treatment with 20 μg TPA per application resulted in tumor development in 2/5 males and 4/4 females.

C. Photosafety Studies

XMP.629 was evaluated for photoallergic potential administered topically to hairless guinea pigs. Primary irritancy, phototoxicity (photoirritancy) and contact hypersensitivity were also evaluated in these studies.

Male CrI:IaF(HA)-hrBR (Outbred) albino hairless guinea pigs were assigned to 7 groups, five guinea pigs per group as outlined below.

| | | Primary Irritancy | | Contact Hypersensitivity | | Contact Hypersensitivity | |
|---|---|---|---|---|---|---|---|
| | | or | | or | | or | |
| | Purpose | Phototoxicity | | Photoallergy Induction | | Photoallergy Challenge | |
| Dosage | For | Formulation | UVR | Formulation | UVR | Formulation | UVR |
| Group | Inclusion^{a.} | Administration | Exposure | Administration | Exposure | Administration | Exposure |
| 1 | Primary | Yes | None | N/A | N/A | N/A | N/A |
| | Irritancy | | | | | | |
| 2, 3 | Phototoxicity | Yes | Yes | N/A | N/A | N/A | N/A |
| 4, 5 | Contact | N/A | N/A | Yes | None | Yes | None |
| | Hypersensitivity | | | | | | |
| 6, 7 | Photoallergy | N/A | N/A | Yes | Yes | Yes | Yes |

^{a}The primary irritancy study phase included test article evaluation only. The phototoxicity phase included test article and comparator article (8-MOP) evaluations. The contact hypersensitivity and photoallergy study phase included test article and comparator article (TCSA) evaluations.
N/A = Not applicable

XMP.629 was formulated as a gel. The phototoxicity comparator article was 8-methoxypsoralen (8-MOP) in methanol at concentrations of 0.1, 0.3 and 1.0 mg/mL. The contact hypersensitivity and photoallergy comparator article was 3,3′,4′,5′-tetrachlorosalicy/anilide (TCSA) in acetone:corn oil (4:1, v/v) at challenge concentrations of 0 (Vehicle), 10, and 30 mg/mL. The reagent and reagent vehicle were Freund's complete adjuvant (FCA) and sterile water for injection, USP, respectively.

The formulation administration and ultraviolet radiation (UVR) exposure regimens for each group in the study were as follows.

| | Primary Irritancy or | | Contact Hypersensitivity or | | Contact Hypersensitivity | |
|---|---|---|---|---|---|---|
| | Phototoxicity | | Photoallergy Induction | | or Photoallergy Challenge | |
| | Formulation | | Formulation | | Formulation | |
| Dosage | Administration | UVR | Administration | UVR | Administration | UVR |
| Group | (mg/mL) | Exposure | (mg/mL) | Exposure | (mg/mL) | Exposure |
| 1 | Test Article^{a. b.} | None | N/A | N/A | N/A | N/A |
| | 0, 1, 10 | | | | | |
| 2 | Test Article^{a. b.} | Yes | N/A | N/A | N/A | N/A |
| | 0, 1, 10 | | | | | |
| 3 | 8-MOP^{c} | Yes | N/A | N/A | N/A | N/A |
| | (0.1, 0.3 and 1.0) | | | | | |
| 4 | N/A | N/A | Test Article^{a.} | None | Test Article^{a. b.} | None |
| | | | (10) | | (0, 1 and 10) | |
| 5 | N/A | N/A | TCSA^{d} | None | TCSA^{d} | None |
| | | | (30) | | (0, 10 and 30) | |
| 6 | N/A | N/A | Test Article^{a.} | Yes | Test Article^{a. b.} | Yes |
| | | | (10) | | (0, 1 and 10) | |
| 7 | N/A | N/A | TCSA^{d} | Yes | TCSA^{d} | Yes |
| | | | (30) | | (0, 10 and 30) | |

^{a}XMP.629 acetate gel
^{b}All three concentrations of XMP.629 acetate gel (0, 1 and 10 mg/mL, corresponding to 0, 0.1 and 1.0%) were administered to each guinea pig.
^{c}8-methoxypsoralen (8-MOP)
^{d}3, 3′, 4′, 5′-tetrachlorosalicylanilide (TCSA)
N/A = Not applicable

For all phases of the study, test articles, test article vehicles, comparator articles and comparator article vehicles were administered (0.3 ml) using a Hilltop chamber affixed to the guinea pig with dental dam overlaid with a Velcro® wrap for approximately 2.0 hours per administration.

Primary irritancy potential of the test articles and test article vehicles was evaluated in Group 1 guinea pigs. Three chambers (one per formulation dosage) were attached to the dorsal skin along the midline. After the administration period the chamber patches were removed and the application sites were gently wiped.

Cutaneous phototoxicity potential of the test articles and test article vehicles was evaluated in Group 2 guinea pigs. Group 3 guinea pigs were used as a comparator group with elicitation of phototoxicity with 8-MOP. Three chambers (one per formulation dosage) were attached to the dorsal skin along the midline and occluded. After the administration period the chamber patches were removed and the application sites were gently wiped. After wiping, Group 2 and 3 guinea pigs were exposed to solar-simulated UVR for approximately 2.25 hours.

For induction of contact hypersensitivity and photoallergy, a nuchal area of skin approximately 2.5 cm² was defined by intradermal injections with a formulation of sterile water and FCA in guinea pigs in Groups 4 through 7 under isoflurane/oxygen anesthesia. This skin area was then tape stripped five times. Formulations were topically administered via chambers as described in the study design table using one chamber attached to the nuchal area. After removal of the chambers the application sites were gently wiped and the nuchal site of Group 6 and 7 guinea pigs was exposed to UVR as above. Appropriate procedures for guinea pigs (with the exception of injection with FCA) were repeated once daily on Days 3, 5, 8, 10 and 12 of the induction phase.

On Day 22 (contact hypersensitivity and photoallergy challenge), formulations were topically applied to appropriate sites on guinea pigs in Groups 4 through 7 as described above. Three chambers (one per formulation dosage) were attached to the dorsal skin along the midline and occluded. The chambers were then removed and the application sites were gently wiped. Guinea pigs in Groups 6 and 7 were then exposed to UVR as above.

All guinea pigs were observed for viability at least twice each day of the study. Observations for general appearance and clinical signs were made weekly throughout the study. Clinical observations of the test article administration sites were made 1, 2 and 3 days after test article administration (primary irritancy and contact hypersensitivity challenge) and test article administration and UVR exposure (phototoxicity and photoallergy challenge). Body weights were recorded at initiation of dosing for each phase, weekly thereafter and at sacrifice. All guinea pigs were sacrificed on the third day after test article administration.

A single topical administration of XMP.629 at concentrations as high as 10 mg/mL (1.0%) in the placebo gel at 0.3 mL/skin site to albino hairless [CrI:IAF(HA)-hrBR (Outbred)] male guinea pigs did not cause skin changes indicative of primary irritation, phototoxicity, contact hypersensitivity or photoallergy. A single topical administration of the comparator article 8-MOP produced skin reactions indicative of cutaneous phototoxicity. Administration of the comparator article TCSA produced skin reactions indicative of contact hypersensitivity and photoallergy. Body weight, body weight changes and clinical observations were unremarkable.

D. Parenteral

Two additional studies, a cardiovascular safety pharmacology test in conscious telemetered cynomolgus monkeys and a series of intravenous efficacy tests in rabbits and rodents, were performed with XMP.629.

D1. Safety Pharmacology Test

This safety pharmacology test was performed to assess the possible cardiovascular effects of XMP.629 when administered via intravenous infusion. The test was an acute experiment to identify single-dose-induced side-effects as indicated, for example, in the Guideline for General Pharmacology Studies (Japan Ministry of Health and Welfare PAB/NND Notification No. 4, Jan. 29, 1995, the disclosure of which is incorporated herein by reference).

A telemetry device/transmitter (Data Sciences, St. Paul, Minn.) was surgically implanted in the subcutaneous pocket over the mid-abdominal region into the test monkeys. The blood pressure catheter of the telemetry device was run sub-cutaneously to the left groin region and inserted into the left femoral artery, with the catheter tip placed into the abdominal aorta. In addition, electrocardiogram leads were sub-cutaneously tunneled to the appropriate anatomical regions (i.e. negative lead paced at the base of the right side of the neck and the positive lead placed within the 5^{th} intercostals space on the left side of the thoracic cage, near the sternum).

Intravenous administration of XMP.629 was given to conscious cynomolgus primates (four males) via telemetry systems in a series of 4 acute experiments, in which escalating doses were administered over four days, with a 3 to 27-day washout period between doses. Doses were administered over a two-hour period, at a rate of 2 mL/kg/hr. The vehicle, 10 mM sodium acetate buffer, was administered on Day 0, and XMP.629 at dose levels of 5, 10, and 20 mg/kg/2 hrs on days 4, 7, and 34, respectively. The infused volume per dose was calculated using the most recent body weight measurements.

Viability checks were performed twice daily on the animals. Body weights were obtained from the animals on the day of surgery and one or two days prior to each dose. Cardiovascular assessments and body temperature measurements were collected radiotelemetrically using the implanted telemetry device/transmitter for one 24 hour period prior to the Day 0 dosing session at the same frequency/periods collected on the days of dosing. On each day of dosing, the effect of XMP.629 on various physiological parameters, such as blood pressure waveform, systolic pressure, diastolic pressure, mean pressure, ECG (axial lead) waveform, heart rate, body temperature, QA interval, P-R interval, QRS interval, QT interval, R—R interval, and QTC interval, was observed two hours before the dosing period, every five minutes for 120 minutes during each infusion period, and for 22 hours after termination of infusion (once every five minutes for one hour and then once every 30 minutes for the remaining 21 hours). In addition, manual ten lead electrocardiograms were collected once pretest and once following the last monitoring period. Blood was obtained in order to perform coagulation studies measuring prothrombin time, activated partial thromboplastin time, and fibrinogen levels were performed prior to infusion and 120 minutes post onset of infusion following the last dose (20 mg/kg/2 hrs). Blood samples were also obtained for the determination of plasma concentrations of XMP.629 at the end of infusion.

Results from this study showed that no adverse, related-effect was observed when XMP.629 is given as an intravenous infusion at 5 mg/kg/2 hrs. Administration of XMP.629 at intravenous doses of 10 or 20 mg/kg/2 hrs to cynomolgus monkeys produced ventricular extrasystoles and premature ventricular beats. One animal had severe clinical signs following infusion of 20 mg/kg/2 hrs which included lethargy, decreased body temperature, and prolongation of the QT and QTc interval. Prolongation in prothrombin and activated partial thromboplastin times along with decreases in fibrinogen were noted in 2 of 4 animals following infusion of 20 mg/kg/2 hrs XMP.629. There were no effects related to XMP.629 on heart rate, QA, P-R, QRS, and R-R intervals following intravenous infusion of 5, 10, or 20 mg/kg/2 hrs of XMP.629.

D2. Intravenous Efficacy Tests in Rabbits

A series of efficacy studies with XMP.629 was conducted where slow IV push or short term (5 to 30 min) intravenous infusions were given through the marginal ear vein at 0.1 to 1.0 mg/mL to rabbits, some of whom were previously infected with Staphylococcus aureus, E. coli, or other bacteria, or Candida albicans. Regardless of infection state or organism, gross macroscopic changes were noted after multiple infusions, such as swelling and hemorrhage into the perivascular space. Due to the swelling, the veins could not be infused after two to five administrations of 1-20 mg/kg/day of XMP.629. Histopathologic examination of hematoxylin and eosin sections revealed the presence of fibrin thrombi, regional hemorrhagic necrosis, edema, regional fibroplasia, acute hemorrhage and necrosis of the vessel wall. Secondary changes in the surrounding tissue included edema, regional fibroplasia, acute hemorrhage, and regional hemorrhagic necrosis, as well as the presence of inflammation. These macroscopic changes were similar to the changes observed in the tail veins of rats and mice in other efficacy studies conducted.

### EXAMPLE 8

### Pharmacokinetics, Distribution and Excretion of XMP.629 in Rat

This example addresses pharmacokinetic properties of intravenously administered XMP.629. Pharmacokinetic studies with either tritiated or non-labeled XMP.629 were conducted in male Sprague-Dawley rats. These studies evaluated the pharmacokinetics, distribution, and elimination of XMP.629 after a single intravenous bolus administration.

A. Pharmacokinetic Studies with [³H]-labeled XMP.629

Three groups of male Sprague-Dawley rats received a 0.3 mg/kg intravenous dose of [³H]-labeled XMP.629 (Amersham, _{Piscataway, N.J.}) via the tail vein (approximately 395 μCi/kg body weight). Rats were allotted to three groups with Group 1 (n=3) used for collection of urine and feces, Group 2 (n=12) used for blood sampling, and Group 3 (n=6) used for whole body autoradiography (WBA). Urine and feces were collected from Group 1 for 72 hours following dosing. Blood samples (Group 2) were collected for 24 hours following dosing. Rats were sacrificed for WBA at selected intervals up to 24 hours after dosing and carcass analysis of residual radioactivity at 72 hours. XMP.629 acetate equivalents were measured using liquid scintillation or autoradiography.

Results showed that plasma concentrations of [³H]-labeled XMP.629 decreased rapidly following administration (-100x within 6 hours), followed by a slower decrease in concentration over time. The pooled data were best fit with a three compartment model with an average half-life of approximately 0.24 hours. The pharmacokinetic parameters obtained from the model fit are shown in Table 24. By 24 hours following dosing, plasma concentrations were approximately 0.005 μg/mL. The alpha and beta half-lives were short (˜2 minutes and ˜45 minutes, respectively) followed by a longer terminal half-life (˜8 hours). Central compartment volume of distribution was low (˜120 mL/kg, which is greater than blood volume but less than extracellular fluid volume). Clearance was low (˜360 mL/hr/kg, which is approximately 10% of liver blood flow).

TABLE 24

| | CL | t_{1/2}α | t_{1/2}β | t_{1/2}γ | t_{1/2} | C₀ | V_{c} | Vₛₛ |
|---|---|---|---|---|---|---|---|---|
| | (g plasma/hr/kg) | (hr) | (hr) | (hr) | (hr) | (μg equivalents/g) | (mL/kg) | (mL/kg) |
| Estimate | 358.7 | 0.032 | 0.77 | 8.3 | 0.23 | 2.49 | 121.0 | 1657 |
| S.E. | 15.5 | 0.007 | 0.08 | 1.2 | 0.02 | 0.35 | 17.0 | 184 |

CL = clearance,
t_{1/2α} = α half-life,
t_{1/2β} = β half-life,
t_{1/2c} = γ half-life,
t_{1/2} = average half-life,
C₀ = extrapolated plasma concentration at time 0,
V_{c} = volume of distribution of the central compartment,
Vₛₛ = volume of distribution at steady state

Whole body autoradiography studies measured the concentration of tritium in tissues at several time points following dosing. From these concentrations, area under the concentration vs. time curve (AUC) was calculated as a measure of overall exposure (see Table 25). Tissue distribution of XMP.629 acetate, as measured by tritium counts, indicated that highly perfused tissues (adrenal gland, liver, thyroid, lung and kidney) contained the greatest drug-equivalent concentrations. In some tissues, tritium concentrations were greater than plasma concentrations, suggesting that the equivalents of ³H-XMP.629 may be distributing into these tissues. Based on tissue AUC, liver, lymph nodes and bone marrow had the greatest overall exposure to equivalents of ³H-XMP.629 acetate.

TABLE 25

| | AUC₀₋ₜₗₐₛₜ |
|---|---|
| Tissue | μg equivalents * hr/g |
| Adrenal | 64.98 |
| Blood | 0.24 |
| Bone marrow | 82.65 |
| Eye uveal tract | 1.14 |
| Kidney | 20.20 |
| Liver | 191.86 |
| Lung | 29.25 |
| Cervical lymph node | 86.07 |
| Popliteal lymph node | 165.30 |
| Renal medulla high | 19.83 |
| Spleen | 60.94 |
| Thyroid | 23.38 |

Tissue distribution of the radioactivity associated with XMP.629 acetate is difficult to interpret given the rapid clearance of drug seen in the pharmacokinetic portion of the study. Tissue distribution of a radiolabel is assumed to be reflective of the parent compound. Preliminary calculations suggest that much of the administered tritium is still present in the body at 72 hours following dosing. However, it is not known if this tritium is associated with parent compound, metabolite, or other tritium-containing degradation products or minor contaminants.

Within 72 hours of dosing, approximately 8.7% (S.D.=0.2%) of the tritium associated with XMP.629 was eliminated in the urine. An estimated 14.8% (S.D.=1.0%) of the tritium associated with XMP.629 acetate was eliminated in the feces. Consistent with this data, an analysis of residual tritium in the carcass at the conclusion of the study indicated that 79.7% (S.D.=13.6%) of the radioactivity remained in the body at 72 hours following dosing.

B. Pharmacokinetic Studies with Non-Labeled XMP.629.

Using non-radiolabeled XMP.629, a pharmacokinetic study was conducted in an analogous manner as described above for ³H-XMP.629. However, in this study, a 0.05 or a 0.2 mg/kg intravenous dose of XMP.629 was administered to 2 males and 2 females rats. Blood samples were collected for 168 hours following dosing and plasma XMP.629 concentrations were measured using liquid chromatography/mass spectrometry (LC/MS).

Results from the collected samples showed that plasma concentrations decreased by approximately 100-fold within 4 hours of dosing. The individual rat concentration data was best described with a two compartment model. The pharmacokinetic parameters obtained from the model fit are shown in Table 26. The alpha half-life is short (˜20 minutes) followed by a longer terminal half-life (-16 hours). A majority of the AUC was associated with the alpha phase suggesting that significant elimination is occurring prior to the distribution equilibrium. Clearance was low (˜320 mL/hr/kg) and the central compartment volume of distribution was comparable to extracellular fluid volume.

Following the 0.05 mg/kg dose, the volume of the central compartment was 203.2 (S.E.=33.6 mL/kg), similar to the value calculated for the 0.2 mg/kg dose.

TABLE 26

| | | | | | | | CL | AUC |
|---|---|---|---|---|---|---|---|---|
| | | t_{1/2} α | t_{1/2} β | t_{1/2} | | | (mL/hr/ | ng*hr/ |
| | V_{c} (mL/kg) | (hr) | (hr) | (hr) | FAUCα | Vₛₛ (mL/kg) | kg) | mL |
| Mean | 201.9 | 0.354 | 16.2 | 0.44 | 0.71 | 2251.5 | 319.0 | 743.6 |
| S.E. | 53.5 | 0.112 | 5.75 | 0.12 | 0.03 | 813.5 | 91.1 | 136.7 |
| C.V. | 52.9 | 63.4 | 71.3 | | 8.1 | 72.3 | 57.1 | 36.8 |

CL = clearance,
t_{1/2α} = α half-life,
t_{1/2β} = β half-life,
FAUCα = fraction of plasma AUC associated with alpha phase,
t_{1/2} = average half-life,
V_{c} = volume of distribution of the central compartment,
Vₛₛ = volume of distribution at steady state.

Results with the non-radiolabeled XMP.629 produced results comparable to those obtained in the radiolabeled XMP.629 study. Pharmacokinetic parameters (e.g. clearance, volume of distribution, and average half-life) were comparable between the two studies (see, e.g., Table 24 and Table 26). The general agreement between these studies, one measuring XMP.629, the other measuring tritium from ³H-XMP.629, suggest that XMP.629 was not extensively metabolized in the rat.

C. Pharmacokinetics of an Oral Dose of XMP.629

This study was designed to determine the pharmacokinetics of XMP.629 in rats following an oral gavage dose. Rats (2 male/2 female per group) received a 10 mg/kg dose of XMP.629 acetate as an oral gavage in either distilled water or a 5% glucose solution. Blood samples were collected at selected times from 1 minute to 168 hours following dosing, plasma was extracted, and XMP.629 levels were measured using LC/MS/MS.

Plasma concentrations following oral dosing were very low, with few concentrations above the limit of detection (1 ng/mL), making the calculation of pharmacokinetic parameters shown in Table 27 difficult. Peak concentrations were achieved 10-20 minutes following dosing, suggesting that absorption was rapid and may be occurring in the stomach. The peak concentrations achieved were ˜4-fold higher with the 5% glucose formulation than with distilled water. Bioavailability was extremely low, (˜0.001%) for XMP.629 acetate in distilled water and ˜4-fold higher with the 5% glucose formulation.

TABLE 27

| | | | |
|---|---|---|---|
| Tₘₐₓ | Cₘₐₓ | AUCₐₗₗ | |
| hours | ng/mL | ng * hr/mL | % F |

Distilled Water

| | | | | |
|---|---|---|---|---|
| Mean | 0.14 | 1.5 | 0.55 | 0.0015 |
| S.D. | 0.05 | 0.3 | 0.55 | 0.0015 |

5% glucose

| | | | | |
|---|---|---|---|---|
| Mean | 0.19 | 4.6 | 1.67 | 0.0044 |
| S.D. | 0.13 | 0.3 | 0.91 | 0.0024 |

AUC = area under the curve.
% F = bioavailability

D. Pharmacokinetics of XMP.629 Following Administration of Topical Dose

This study was designed to determine the pharmacokinetics of XMP.629 in rats following a topical dose of XMP.629 acetate gel. Forty-eight rats were assigned to three treatment groups (8 male/8 female per group) with each group to receive a different strength of XMP.629 acetate gel. Gel was applied (0.1, 0.5 or 1.0% acetate salt by weight) at a rate of 2 mg of gel/cm2 to a shaved area of approximately 15% of the total body surface area. Gel was allowed to air dry for 20-30 minutes before animals were returned to individual housing. Each rat was sampled at 3 selected times between 8 and 28 hours following dose application. Blood samples were centrifuged to obtain plasma and plasma concentrations of XMP.629 were determined using LC/MS/MS.

Plasma concentrations following topical dosing were very low, with no concentrations above 3 ng/mL following dosing with a 0.1% gel. In the 0.5% gel dose group there was a single sample (3.5 ng/mL at 6 hours) containing a measurable concentration of XMP.629. At the highest dose, 1.0% gel, approximately 60% of the samples were below the limit of detection (1 ng/mL), two values in this group were greater than 19 ng/mL and were considered spurious, and the remaining samples were <7.5 ng/mL. The scarcity of above detection level data in all groups complicated the calculation of pharmacokinetic parameters and was attempted only with the data from the 1.0% gel formulation. The highest concentrations were estimated to occur between 30 and 120 minutes following application of the gel. The bioavailability of XMP.629 acetate following the application of the 1.0% gel was about approximately 0.29%, which was determined by assigning all bql values a concentration of ½ bql (0.5 ng/mL).

E. Pharmacokinetics of XMP.629 Following Administration of an Intravenous Dose

This study was designed to determine the pharmacokinetics of XMP.629 in Sinclair mini-pigs following an intravenous bolus or topical dose. Adult Sinclair mini-pigs (2M/2F per dose group, in a cross-over design) received approximately 0.05 or 2 mg/kg XMP.629 acetate as a short IV infusion into a catheterized ear vein. Blood samples were collected at selected times for up to 168 hours after dosing, plasma was extracted and assayed for XMP.629 using an LC/MS/MS assay (limit of quantification=1 ng/mL).

Plasma XMP.629 levels declined rapidly with a 100-fold decrease within the first 12 hours. The initial half-life was 0.03-0.9 hours, with a beta half-life of 1-1.3 hours, and a terminal half-life of approximately 15 hours as shown in Table 28. The central compartment volume of distribution was similar to blood volume (67-102 mL/kg) and the plasma clearance (105-156 mL/hr/kg) was a small fraction of hepatic or renal plasma flow.

TABLE 28

| | | T_{1/4} | | | | | | |
|---|---|---|---|---|---|---|---|---|
| | V_{c} | average | T_{1/2}α | T_{1/2}β | T_{1/2}γ | MRT | V₃₅ | CL |
| Dose | [mL/kg] | [hr] | [hr] | [hr] | [hr] | [hr] | [mL/kg] | {mL/hr/kg} |
| 0.05 mg/kg | 67.4 | 0.36 | 0.091 | 1.28 | 15.0 | 11.26 | 1793 | 155.7 |
| | (11.0) | (0.076) | (0.05) | (0.53) | (nc) | (1.67) | (635.5) | (52.21) |
| 0.2 mg/kg | 101.8 | 0.58 | 0.031 | 1.03 | 15.4 | 7.41 | 956 | 104.6 |
| | (54.1) | (0.097) | (0.01) | (0.22) | (2.53) | (1.73) | (557.3) | (36.96) |

Nc = not calculated
MRT = mean residence time

XMP.629 acetate gel (0.1, 0.5, or 1% XMP.629 acetate per gram gel) was applied to the skin of mini-pigs. The dose of gel in each group was calculated as that amount which would cover 15% of the body surface area (BSA) at a rate of 2 mg of gel per cm2 of BSA. The application area was washed 24 hours following XMP.629 acetate gel application. Blood samples were collected at selected times up to 168 hours after dosing, plasma was extracted, and assayed for XMP.629 using an LC/MS/MS assay (limit of quantification=1 ng/mL).

Bioavailability of XMP.629 following topical administration of XMP.629 was very low, with no measurable plasma concentrations.

### EXAMPLE 9

### Acne Clinical Study with XMP.629

The safety and efficacy of XMP.629 is investigated in human clinical studies. Subjects with acne were selected and administered a composition of XMP.629 as described in Example 5 (see Table 12).

A. Skin Irritation Study.

A dermal study to determine the cumulative skin irritation potential of XMP.629 is conducted at a single center. Specifically, in a 21-day evaluator-blind assessment, gels comprising either XMP.629 (0.1%, acetate salt), vehicle gel, or 0.2% sodium lauryl sulfate are repeatedly applied to the skin of 35 healthy subjects. Subjects include males and females, ranging in age from 18 to 70 years old. Subjects are of any skin and race type.

Absorbent pads (19 mm diameter) comprising 0.2 mL of either XMP.629 (0.1%, acetate salt), vehicle gel, or 0.2% sodium lauryl sulfate are prepared as patches using Hill Top Chambers (25 mm diameter). Patches are prepared approximately 5-60 minutes before application to the subject and are applied on the backs of subjects at designated sites. Patches are held in place with appropriate adhesion tapes for 48 hours. Gels are applied to the same site on a given subject according to the randomization code three times per week. Subjects are instructed to keep the patches dry and are asked to avoid exercise that results in excessive sweating. Additionally, subjects are instructed to not expose the applied sites to sunlight for the duration of the study.

Every application is observed 48 hours later for signs of irritation or inflammation by a clinician or investigator. The clinician or investigator evaluates sign of skin reaction on each site for approximately 5-15 minutes after the patch is removed. Skin irritancy is rated according to the following commonly used 6-point scale:
0=No sign of irritation
0.5=Barely perceptible erythema
1=Slight erythema
2=Noticeable erythema with slight infiltration
3=Erythema with marked edema
4=Erythema with edema and blistering

Other signs of skin reaction to the tested gels are noted as adverse events and are recorded. A total of nine readings are performed during the study.

Mean irritation scores and their frequency distribution are tabulated by site and evaluation day. Mean scores are summed across days for each site. The cumulative irritation score for each site corresponds to the total irritation score divided by the highest theoretical score. When a particular site is discontinued due to severity of irritation (Grade 4), the last observation for that site is carried forward. Parameters are tested pair-wise for product differences using Fisher's protected least significant differences in the context of the two-way variance analysis (ANOVA), including main effect of subject and product without interaction.

The majority of the adverse events observed in the study were pruritus or related application site reactions, consistent with occlusive patching and application of a positive control for irritation. Overall, XMP.629 was well tolerated by the subjects in this study.

The mean cumulative irritation score of the positive control (sodium lauryl sulfate, 0.3%) was 0.25, which was significantly more irritating than XMP.629 acetate gel, 0.1% (mean 0.08) and drug product vehicle gel (mean 0.07) (p<0.001). There was no significant difference in cumulative irritation between XMP.629 acetate gel, 0.1% and drug product vehicle gel (p=0.592). Under the conditions of the study, XMP.629 acetate gel, 0.1% and drug product vehicle gel produced no significant cumulative irritation; sodium lauryl sulfate (0.3%), the positive control, produced mild cumulative irritation.

The cumulative irritation and adverse event safety results of this study indicated that XMP.629 acetate gel, 0.1% is well tolerated and does not produce significant irritation as compared to a positive control for mild irritation.

B. Absorption Study

An absorption evaluation study was conducted following maximum topical exposure to XMP.629 acetate gel, 0.1%, in 15 subjects with moderate to very severe acne.

The study population consisted of males and females, 12 years of age and older with moderate to very severe acne vulgaris, defined as having a total of approximately 1,000 to 2,500 cm² of acne-involved area on the face, chest and back and an Evaluator's Global Severity Scale score of 3 to 5 on a 0 to 5 scale in a least one of these three areas.

The objective of this study was to investigate the absorption and safety of XMP.629 acetate gel, 0.1%, following maximal exposure using multiple applications in subjects with moderate to very severe acne. Preliminary efficacy was assessed through lesion counts and Evaluator's Global Severity Scale evaluations of acne vulgaris.

Subjects selected with acne as described above came to the clinic daily for 14 days and study staff applied 4 g of XMP.629 acetate gel, 0.1%, to affected areas of the face and trunk, e.g., one application daily for 14 days for a total of 14 applications. This daily dose represented an 8-fold increase over the anticipated typical upper clinical dose. Pre-dose blood samples were drawn on Days 1, 5, 10 and 14. On Days 1 and 14, blood was also drawn at 15 minutes, 30 minutes, and 1, 2, 3, 6 and 9 hours post-dosing. A final 24-hour post-dosing blood draw was taken on Day 15. Serum was analyzed to determine the concentrations of XMP.629 and antibodies against XMP.629.

Pharmacokinetic parameters for serum concentrations of XMP.629 were determined on Day 1 and Day 14. Mean, standard deviation and coefficient of variation (CV) were to be calculated for each of these parameters. An assessment of changes in pharmacokinetic parameters from Day 1 to Day 14 was to be made, if possible. Trough XMP.629 serum levels were to be measured pre-dose on Days 1, 5, 10 and 14. A comparison of these trough concentrations were conducted. Since XMP.629 serum concentrations were below the limit of detection (e.g., 1 ng/mL), the pharmacokinetic analyses were not performed.

Three subjects reported four adverse events of which one, mild dry skin, was considered related to study drug. The adverse events were mild and reversible. There were no clinically significant changes in laboratory tests, vital signs or physical exam findings.

A non-therapeutic assessment of cutaneous safety, including assessment of dermatological/cosmetic effects (e.g. scaling, erythema, burning, stinging and itching) was measured using the following scale described in Table 29.

TABLE 29

| Score | Grade | Description |
|---|---|---|
| Scaling | | |
| 0 | None | No scaling |
| 1 | Mild | Barely perceptible, fine scales present to limited |
| | | areas of the face |
| 2 | Moderate | Fine scale generalized to all areas of face |
| 3 | Severe | Scaling and peeling of skin over all areas of the |
| | | face |
| Erythema | | |
| 0 | None | No evidence of erythema present |
| 1 | Mild | Slight pink coloration |
| 2 | Moderate | Definite redness |
| 3 | Severe | Marked erythema, bright red to dusky dark red in |
| | | color |
| Itching | | |
| 0 | None | No itching |
| 1 | Mild | Slight itching, not really bothersome |
| 2 | Moderate | Definite itching that is somewhat bothersome |
| 3 | Severe | Intense itching that may interrupt daily activities |
| | | and/or sleep |
| Burning | | |
| 0 | None | No burning |
| 1 | Mild | Slight burning sensation; not really bothersome |
| 2 | Moderate | Definite warm, burning sensation that is |
| | | somewhat bothersome |
| 3 | Severe | Hot burning sensation that causes definite |
| | | discomfort and may interrupt daily activities |
| | | and/or sleep |
| Stinging | | |
| 0 | None | No stinging |
| 1 | Mild | Slight stinging sensation, not really bothersome |
| 2 | Moderate | Definite stinging sensation that is somewhat |
| | | bothersome |
| 3 | Severe | Stinging sensation that causes definite discomfort |
| | | and may interrupt daily activities and/or sleep |

Assessments were made at baseline, Day 10 and Day 14 and are shown in Table 30. The results demonstrated that treatment was not associated with any scaling, itching, burning and stinging. In fact, treatment reduced mild (n=10) and moderate (n=2) erythema at baseline to zero at Day 14. In addition, mild itching present in three subjects at baseline was resolved at Day 14.

TABLE 30

| | Baseline | Day 10 | Day 14 |
|---|---|---|---|
| | (N = 15) | (N = 15) | (N = 15) |
| Scaling | | | |
| None (0) | 14 (93%) | 15 (100%) | 14 (93%) |
| Mild (1) | 1 (7%) | 0 (0%) | 1 (7%) |
| Moderate (2) | 0 (0%) | 0 (0%) | 0 (0%) |
| Severe (3) | 0 (0%) | 0 (0%) | 0 (0%) |
| Mean (SD) | 0.07 (0.26) | 0.00 (0.00) | 0.07 (0.26) |
| Erythema | | | |
| None (0) | 3 (20%) | 13 (87%) | 15 (100%) |
| Mild (1) | 10 (67%) | 2 (13%) | 0 (0%) |
| Moderate (2) | 2 (13%) | 0 (0%) | 0 (0%) |
| Severe (3) | 0 (0%) | 0 (0%) | 0 (0%) |
| Mean (SD) | 0.93 (0.59) | 0.13 (0.35) | 0.00 (0.00) |
| Itching | | | |
| None (0) | 12 (80%) | 14 (93%) | 15 (100%) |
| Mild (1) | 3 (20%) | 1 (7%) | 0 (0%) |
| Moderate (2) | 0 (0%) | 0 (0%) | 0 (0%) |
| Severe (3) | 0 (0%) | 0 (0%) | 0 (0%) |
| Mean (SD) | 0.20 (0.41) | 0.07 (0.26) | 0.00 (0.00) |
| Burning | | | |
| None (0) | 15 (100%) | 15 (100%) | 15 (100%) |
| Mild (1) | 0 (0%) | 0 (0%) | 0 (0%) |
| Moderate (2) | 0 (0%) | 0 (0%) | 0 (0%) |
| Severe (3) | 0 (0%) | 0 (0%) | 0 (0%) |
| Mean (SD) | 0.00 (0.00) | 0.00 (0.00) | 0.00 (0.00) |
| Stinging | | | |
| None (0) | 15 (100%) | 15 (100%) | 15 (100%) |
| Mild (1) | 0 (0%) | 0 (0%) | 0 (0%) |
| Moderate (2) | 0 (0%) | 0 (0%) | 0 (0%) |
| Severe (3) | 0 (0%) | 0 (0%) | 0 (0%) |
| Mean (SD) | 0.00 (0.00) | 0.00 (0.00) | 0.00 (0.00) |

Preliminary therapeutic activity was assessed through lesion count and Evaluator's Global Severity Scale evaluations (using a scale of 0, clear, to 5, very severe). Safety was assessed through vital signs, clinical laboratory tests, physical exam and the occurrence of adverse events.

Preliminary therapeutic efficacy results indicated a 33% decrease from baseline in mean inflammatory lesion count, a 28% decrease in mean non-inflammatory lesion count and a 30% decrease in mean total lesion count at Day 15. Eleven out of fifteen patients (73%) had at least a 1 grade improvement from baseline in their Evaluator Global Severity Score in either face, back, or chest at Day 15. In this study, 53% of patients had at least a 1 grade improvement from baseline in the Evaluator Global Severity Scale in the face, 40% on the back and 33% on the chest. Thus, XMP.629 acetate gel, 0.1%, was well tolerated, lacked a measurable systemic absorption and demonstrated a reduction in baseline erythema, lesion count and Evaluator's Global Severity Scale, as shown in this study after 14 days of treatment.

C. Efficacy Study

A clinical study using XMP.629 for the treatment of acne is conducted at multiple centers. Specifically, a double-blind study is performed on patients with acne vulgaris following once a day repeated topical application with a gel comprising XMP.629 at 0.01%, 0.05%, and 0.1%.

The subject population is male or female that is 12 years of age and older and exhibits mild to moderate acne vulgaris. The study is conducted over a 12-week period.

After 12 weeks, the following criteria are evaluated to determine efficacy:
(1) percent reduction in inflammatory lesion count, including the inflammatory facial lesion count;
(2) percent reduction in non-inflammatory lesion count, including the non-inflammatory facial lesion count;
(3) percent reduction in total lesion count, including the total facial lesion count; and
(4) proportion of subjects judged as clear or almost clear, based on a Global Static Physician score or an Evaluator Global Severity Scale, including in the face.

A primary endpoint is the percent reduction in inflammatory lesion count. An alternative primary endpoint is the mean reduction from baseline in inflammatory lesion count, non-inflammatory lesion counts and/or total lesion counts, and/or the proportion of subjects judged as clear or almost clear based on an Evaluator Global Severity Scale, preferably by assessing facial lesion counts and facial skin clearance. A secondary objective is the percent reduction in non-inflammatory lesion counts and total lesion counts, and the proportion of subjects judged as clear or almost clear based on a Global Static Physician score (success). An analysis of variance is used to test the treatment effect for the percent reduction in inflammatory, noninflammatory, and total lesion counts. Contrasts are used to make pairwise comparisons between the treatment groups. A PROC CATMOD analysis with a factor of treatment is used to test the proportion of subjects considered a success. Contrasts within this procedure are used for pairwise comparisons. Analysis of variance, Cochran-Mantel-Haenszel tests, and rank tests are used to analyze these endpoints, as appropriate. The efficacy of XMP.629 is indicated by a reduction of one or more of the four criteria evaluated.

Efficacy assessments are thus based on blinded investigator assessments of amelioration of acne, including by assessing one or more of the signs and symptoms of acne vulgaris and including where the amelioration is indicated by one or more of the following: reduction in inflammatory lesion count (e.g., facial), reduction in non-inflammatory lesion count (e.g., facial), reduction in total lesion count (e.g., facial) or an increased proportion of clear or almost clear skin (e.g., facial). A reduction in lesion counts (e.g., inflammatory, non-inflammatory and/or total) may be analyzed as final lesion counts, changes from baseline (e.g., baseline counts—end of study counts) or percent change (e.g., change/baseline counts×100%). An increased proportion of clear or almost clear skin is analyzed in a variety of ways by a trained evaluator (e.g., physician or investigator), preferably a physincan global evaluation, including using a Global Static Physician Score, Static physician Global Assessment, Investigator Global Evaluation, Evaluator's Global Severity Scale, or other known scale (e.g., Cook's Scale, Leeds Scale, etc.). A primary measure of efficacy variables percent change from baseline as week 12 in (i) inflammatory lesion counts, (ii) non-inflammatory lesion counts, and (iii) total lesion counts, including, for example, where the counts are facial. A secondary measurement of efficacy is the percent of subjects who are clear or almost clear, for example, at week 12, as judged by an Evaluator's Global Severity Score as shown in Table 31.

TABLE 31

| Score | Grade | Description |
|---|---|---|
| 0 | Clear | Normal, clear skin with no evidence of acne |
| | | vulgaris |
| 1 | Almost Clear | Rare non-inflammatory lesions present, with |
| | | rare non-inflamed papules (papules must be |
| | | resolving and may be hyperpigmented, though |
| | | not pink-red) |
| 2 | Mild | Some non-inflammatory lesions are present, |
| | | with few inflammatory lesions |
| | | (papules/pustules only; no nodulo-cystic |
| | | lesions). |
| 3 | Moderate | Non-inflammatory lesions predominate, with |
| | | multiple inflammatory lesions evident; several |
| | | to many comedones and papules/pustules, and |
| | | there may or may not be one small nodulo- |
| | | cystic lesion |
| 4 | Severe | Inflammatory lesions are more apparent: many |
| | | comedones and papules/pustules; there may |
| | | or may not be a few nodulo-cystic lesions |
| 5 | Very Severe | Highly inflammatory lesions predominate; |
| | | variable number of comedones, many |
| | | papules/pustules and nodulo-cystic lesions |

Safety is evaluated by vital signs, clinical laboratory tests, detection of antibodies against XMP.629, physical exam findings, Cutaneous Safety Evaluation scores (erythema and scaling), as well as the Tolerability Evaluation scores (itching, burning, and stinging) and by the incidence of adverse events reported.

An exemplary study population for efficacy assessment consists of males and females, 12 years of age and older, with mild to moderate acne vulgaris, defined as having facial acne with inflammatory lesion (papules and pustules) counts of 20 to 50, non-inflammatory lesion (open and closed comedones) counts of 100 or less, one or fewer nodules (defined as an inflammatory lesion greater than or equal to 5 mm in diameter) and/or an Evaluator's Global Severity Score of 2 or 3 (see Table 31). The efficacy of XMP.629 is indicated by amelioration of acne, including where the amelioration is indicated by a reduction in the number and/or severity of one or more signs or symptoms of acne, including, for example, a reduction (e.g., decrease) in lesion counts and/or an improvement (e.g., increase) in the clear or almost clear skin, assessed after treatment with XMP.629 as described above.

All U.S. Patents, U.S. Patent Applications, International PCT Applications, and references cited herein are hereby incorporated by reference in their entirety. While the invention will be described in connection with one or more embodiments, it will be understood that the invention is not limited to those embodiments. On the contrary, the invention includes all alternatives, modification, and equivalents as may be included within the spirit and scope of the appended claims.

1. A method of treating acne comprising administering to a subject with acne a composition comprising a therapeutically effective amount of XMP.629 or a pharmaceutically acceptable salt or derivative thereof.

2. The method of claim 1, wherein the subject has mild or moderate acne.

3. The method of claim 1, wherein the pharmaceutically acceptable salt is acetate.

4. The method of claim 1, wherein the composition further comprises at least one additive.

5. The method of claim 4, wherein the additive is a chelating agent, tonicity agent, gelling agent, buffer, surfactant, or preservative.

6. The method of claim 1, wherein the composition is in an oral, parental, or topical formulation.

7. The method of claim 6, wherein the composition is in a topical formulation.

8. The method of claim 7, wherein the topical formulation is a cream, gel, lotion, or solution.

9. The method of claim 7, wherein the topical formulation is a cream.

10. The method of claim 7, wherein the topical formulation is a gel.

11. The method of claim 7, wherein the topical formulation is a lotion.

12. The method of claim 7, wherein the topical formulation is a solution.

13. The method of claim 7, wherein the topical formulation is presented in an impregnated dressing.

14. The method of claim 7, wherein the topical formulation is presented in a patch.

15. The method of claim 7, wherein the topical formulation is presented in a gel stick.

16. The method of claim 7, wherein the topical formulation is presented in a spray.

17. The method of claim 7, wherein the topical formulation is presented in an aerosol.

18. The method of claim 7, wherein the topical formulation is presented in a wipe.

19. The method of claim 7, wherein the topical formulation is presented in a swab.

20. The method of claim 1, wherein the therapeutically effective amount of XMP.629 or a pharmaceutically acceptable salt or derivative thereof ranges from about 0.005% to about 0.5% (weight to volume).

21. The method of claim 20, wherein the therapeutically effective amount of XMP.629 or a pharmaceutically acceptable salt or derivative thereof ranges from about 0.01% to about 0.2% (weight to volume).

22. The method of claim 21, wherein the therapeutically effective amount of XMP.629 or a pharmaceutically acceptable salt or derivative thereof ranges from about 0.01% to about 0.1% (weight to volume).

23. The method of claim 22, wherein the therapeutically effective amount of XMP.629 or a pharmaceutically acceptable salt or derivative thereof ranges from about 0.05% to about 0.1% (weight to volume).

24. The method of claim 20, wherein the therapeutically effective amount of XMP.629 or a pharmaceutically acceptable salt or derivative thereof is about 0.01% (weight to volume).

25. The method of claim 20, wherein the therapeutically effective amount of XMP.629 or a pharmaceutically acceptable salt or derivative thereof is about 0.05% (weight to volume).

26. The method of claim 20, wherein the therapeutically effective amount of XMP.629 or a pharmaceutically acceptable salt or derivative thereof is about 0.1% (weight to volume).

27. The method of claim 1, wherein administration of the composition is performed from about once to about five times a day.

28. The method of claim 27, wherein administration of the composition is performed from about three to about four times a day.

29. The method of claim 27, wherein administration of the composition is performed about once a day.

30. The method of claim 1, wherein the acne is ameliorated as indicated by at least one of the following: reduction in inflammatory lesion count; reduction in non-inflammatory lesion count; reduction in total lesion count; or an increased proportion of clear or almost clear skin.

31. The method of claim 1, further comprising additionally administering to the subject at least one anti-acne agent, wherein the anti-acne agent is not XMP.629 or a pharmaceutically acceptable salt or derivative thereof.

32. The method of claim 31, wherein the anti-acne agent is in a topical formulation.

33. The method of claim 32, wherein the topical formulation is a cream, gel, lotion, or solution.

34. The method of claim 32, wherein the topical formulation is a cream.

35. The method of claim 32, wherein the topical formulation is a gel.

36. The method of claim 32, wherein the topical formulation is a lotion.

37. The method of claim 32, wherein the topical formulation is a solution.

38. The method of claim 32, wherein the topical formulation is presented in an impregnated dressing.

39. The method of claim 32, wherein the topical formulation is presented in a patch.

40. The method of claim 32, wherein the topical formulation is presented in a gel stick.

41. The method of claim 32, wherein the topical formulation is presented in a spray.

42. The method of claim 32, wherein the topical formulation is presented in an aerosol.

43. The method of claim 32, wherein the topical formulation is presented in a wipe.

44. The method of claim 32, wherein the topical formulation is presented in a swab.

45. The method of claim 31, wherein the anti-acne agent is a prescription based or over-the-counter agent.

46. The method of claim 45, wherein the prescription based anti-acne agent is selected from one of the following: benzoyl peroxide, retinoids, retinoid derivatives, antimicrobial agents, or combinations thereof.

47. The method of claim 46, wherein the benzoyl peroxide is administered in an amount ranging from about 2.5% to about 10%.

48. The method of claim 46, wherein the retinoid is tretinoin.

49. The method of claim 47, wherein the tretinoin is administered in an amount ranging from about 0.01% to about 0.025%.

50. The method of claim 46, wherein the retinoid derivative is acetylenic retinoid or naphtholic acid retinoid.

51. The method of claim 46, wherein the antimicrobial agent is azelaic acid.

52. The method of claim 46, wherein the antibiotic is clindamycin, tetracycline, doxycycline, or erythromycin.

53. The method of claim 46, wherein the antibiotic combination is benzoyl peroxide with clindamycin, tetracycline, doxycycline, or erythromycin.

54. The method of claim 51, wherein the benzoyl peroxide is administered in an amount ranging from about 2.5% to about 10%.

55. The method of claim 1, wherein the composition is for repeated administration.

56. A method of treating acne comprising:
(a) selecting a subject with acne; and
(b) administering to the subject a composition comprising a therapeutically effective amount of XMP.629 or a pharmaceutically acceptable salt or derivative thereof.

57. A method for ameliorating acne comprising administering to a subject a composition comprising a therapeutically effective amount of XMP.629 or a pharmaceutically acceptable salt or derivative thereof, wherein the amelioration is indicated by at least one of the following: reduction in inflammatory lesion count; reduction in non-inflammatory lesion count; reduction in total lesion count; or an increased proportion of clear or almost clear skin.

58. The method of claim 57, wherein the subject is a human.

59. The method of claim 57, wherein the pharmaceutically acceptable salt is acetate.

60. The method of claim 57, wherein the composition further comprises at least one additive.

61. The method of claim 60, wherein the additive is a chelating agent, tonicity agent, gelling agent, buffer, surfactant, or preservative.

62. The method of claim 57, wherein the composition is in an oral, parental, or topical formulation.

63. The method of claim 62, wherein the composition is in a topical formulation.

64. The method of claim 63, wherein the topical formulation is a cream, gel, lotion, or solution.

65. The method of claim 63, wherein the topical formulation is a cream.

66. The method of claim 63, wherein the topical formulation is a gel.

67. The method of claim 63, wherein the topical formulation is a lotion.

68. The method of claim 63, wherein the topical formulation is a solution.

69. The method of claim 63, wherein the topical formulation is presented in an impregnated dressing.

70. The method of claim 63, wherein the topical formulation is presented in a patch.

71. The method of claim 63, wherein the topical formulation is presented in a gel stick.

72. The method of claim 63, wherein the topical formulation is presented in a spray.

73. The method of claim 63, wherein the topical formulation is presented in an aerosol.

74. The method of claim 63, wherein the topical formulation is presented in a wipe.

75. The method of claim 63, wherein the topical formulation is presented in a swab.

76. The method of claim 57, wherein the therapeutically effective amount of XMP.629 or a pharmaceutically acceptable salt or derivative thereof ranges from about 0.005% to about 0.5% (weight to volume).

77. The method of claim 76, wherein the therapeutically effective amount of XMP.629 or a pharmaceutically acceptable salt or derivative thereof ranges from about 0.01% to about 0.2% (weight to volume).

78. The method of claim 77, wherein the therapeutically effective amount of XMP.629 or a pharmaceutically acceptable salt or derivative thereof ranges from about 0.01% to about 0.1% (weight to volume).

79. The method of claim 78, wherein the therapeutically effective amount of XMP.629 or a pharmaceutically acceptable salt or derivative thereof ranges from about 0.05% to about 0.1% (weight to volume).

80. The method of claim 76, wherein the therapeutically effective amount of XMP.629 or a pharmaceutically acceptable salt or derivative thereof is about 0.01% (weight to volume).

81. The method of claim 79, wherein the therapeutically effective amount of XMP.629 or a pharmaceutically acceptable salt or derivative thereof is about 0.05% (weight to volume).

82. The method of claim 79, wherein the therapeutically effective amount of XMP.629 or a pharmaceutically acceptable salt or derivative thereof is about 0.1% (weight to volume).

83. The method of claim 57, wherein administration of the composition is performed from about once to about five times a day.

84. The method of claim 83, wherein administration of the composition is performed from about three to about four times a day.

85. The method of claim 84, wherein administration of the composition is performed about once a day.

86. The method of claim 57, further comprising additionally administering to the subject one or more anti-acne agents, wherein the anti-acne agent is not XMP.629 or a pharmaceutically acceptable salt or derivative thereof.

87. The method of claim 86, wherein the anti-acne agent is in a topical formulation.

88. The method of claim 87, wherein the topical formulation is a cream, gel, lotion, or solution.

89. The method of claim 87, wherein the topical formulation is a cream.

90. The method of claim 87, wherein the topical formulation is a gel.

91. The method of claim 87, wherein the topical formulation is a lotion.

92. The method of claim 87, wherein the topical formulation is a solution.

93. The method of claim 87, wherein the topical formulation is presented in an impregnated dressing.

94. The method of claim 87, wherein the topical formulation is presented in a patch.

95. The method of claim 87, wherein the topical formulation is presented in a gel stick.

96. The method of claim 87, wherein the topical formulation is presented in a spray.

97. The method of claim 87, wherein the topical formulation is presented in an aerosol.

98. The method of claim 87, wherein the topical formulation is presented in a wipe.

99. The method of claim 87, wherein the topical formulation is presented in a swab.

100. The method of claim 57, wherein the anti-acne agent is a prescription based or over-the-counter agent.

101. The method of claim 100, wherein the prescription based anti-acne agent is selected from one of the following: benzoyl peroxide, retinoids, retinoid derivatives, antimicrobial agents, or combinations thereof.

102. The method of claim 101, wherein the benzoyl peroxide is administered in an amount ranging from about 2.5% to about 10%.

103. The method of claim 101, wherein the retinoid is tretinoin.

104. The method of claim 103, wherein the tretinoin is administered in an amount ranging from about 0.01% to about 0.025%.

105. The method of claim 101, wherein the retinoid derivative is acetylenic retinoid or naphtholic acid retinoid.

106. The method of claim 101, wherein the antimicrobial agent is azelaic acid.

107. The method of claim 101, wherein the antibiotic is clindamycin, tetracycline, doxycycline, or erythromycin.

108. The method of claim 101, wherein the antibiotic combination is benzoyl peroxide with clindamycin, tetracycline, doxycycline, or erythromycin.

109. The method of claim 108, wherein the benzoyl peroxide is administered in an amount ranging from about 2.5% to about 10%.

110. The method of claim 57, wherein the composition is for repeated administration.

111. A method for ameliorating acne comprising:
(a) selecting a subject with acne; and
(b) administering to the subject a composition comprising a therapeutically effective amount of XMP.629 or a pharmaceutically acceptable salt or derivative thereof, wherein the amelioration is indicated by at least one of the following: reduction in inflammatory lesion count; reduction in non-inflammatory lesion count; reduction in total lesion count; or an increased proportion of clear or almost clear skin.

112. A method of treating acne comprising concurrently administering to a subject with acne (i) a composition comprising a therapeutically effective amount of XMP.629 or a pharmaceutically acceptable salt or derivative thereof; and (ii) at least one anti-acne agent, wherein the anti-acne agent is not XMP.629 or pharmaceutically acceptable salt or derivative thereof, wherein the acne is ameliorated by concurrent administration of (i) and (ii).

113. The method of claim 112, wherein the XMP.629 or pharmaceutically acceptable salt or derivative thereof is administered before or after the anti-acne agent.

114. The method of claim 112, wherein amelioration of the acne is indicated by at least one of the following: reduction in inflammatory lesion count; reduction in non-inflammatory lesion count; reduction in total lesion count; or an increased proportion of clear or almost clear skin.

115. The method of claim 114, wherein the amelioration is indicated by a reduction in inflammatory lesion count.

116. The method of claim 114, wherein the amelioration is indicated by a reduction in non-inflammatory lesion count.

117. The method of claim 114, wherein the amelioration is indicated by a reduction in total lesion count.

118. The method of claim 114, wherein the amelioration is indicated by an increased proportion of clear or almost clear skin.

119. The method of claim 112, wherein the anti-acne agent is a prescription based or over-the-counter agent.

120. The method of claim 119, wherein the prescription based anti-acne agent is selected from one of the following: benzoyl peroxide, retinoids, retinoid derivatives, antimicrobial agents, or combinations thereof.

121. The method of claim 120, wherein the benzoyl peroxide is administered in an amount ranging from about 2.5% to about 10%.

122. The method of claim 120, wherein the retinoid is tretinoin.

123. The method of claim 122, wherein the tretinoin is administered in an amount ranging from about 0.01% to about 0.025%.

124. The method of claim 120, wherein the retinoid derivative is acetylenic retinoid or naphtholic acid retinoid.

125. The method of claim 120, wherein the anti-acne agent is an antimicrobial agent.

126. The method of claim 125, wherein concurrent administration of (i) and (ii) provides enhanced therapeutic effectiveness or increased potency of the anti-acne agent.

127. The method of claim 125, wherein concurrent administration of (i) and (ii) provides synergistic or potentiating effects beyond the individual or additive effects of XMP.629 or the physiologically or pharmaceutically acceptable salt or derivative thereof and the anti-acne agent alone.

128. The method of claim 125, wherein the XMP.629 or physiologically or pharmaceutically acceptable salt or derivative thereof and the anti-acne agent are in amounts that would be sufficient for monotherapeutic effectiveness.

129. The method of claim 125, wherein the XMP.629 or physiologically or pharmaceutically acceptable salt or derivative thereof and the anti-acne agent are in less than monotherapeutically effective amounts.

130. The method of claim 125, wherein concurrent administration of (i) and (ii) provides for decreasing the dose of the anti-acne agent.

131. The method of claim 125, wherein concurrent administration of (i) and (ii) provides for expediting commencement of a therapeutic benefit by the anti-acne agent.

132. The method of claim 125, wherein concurrent administration of (i) and (ii) provides for decreasing the duration of treatment by the anti-acne agent.

133. The method of claim 125, wherein concurrent administration of (i) and (ii) provides for reducing one or more side effects associated with the anti-acne agent.

134. The method of claim 125, wherein the antimicrobial agent is azelaic acid.

135. The method of claim 125, wherein concurrent administration of (i) and (ii) provides a more effective or enhanced potency.

136. The method of claim 125, wherein concurrent administration of (i) and (ii) provides for reduction of resistance in acne-associated bacteria.

137. The method of claim 125, wherein concurrent administration of (i) and (ii) provides for reversal of resistance in acne-associated bacteria.

138. The method of claim 120, wherein the anti-acne agent is an antibiotic.

139. The method of claim 138, wherein the antibiotic is clindamycin, tetracycline, doxycycline, or erythromycin.

140. The method of claim 138, wherein concurrent administration of (i) and (ii) provides a more effective or enhanced potency.

141. The method of claim 138, wherein concurrent administration of (i) and (ii) provides for reduction of resistance in acne-associated bacteria.

142. The method of claim 138, wherein concurrent administration of (i) and (ii) provides for reversal of resistance in acne-associated bacteria.

143. The method of claim 120, wherein the anti-acne agent is an antibiotic combination.

144. The method of claim 143, wherein the antibiotic combination is benzoyl peroxide with clindamycin, tetracycline, doxycycline, or erythromycin.

145. The method of claim 143, wherein concurrent administration of (i) and (ii) provides a more effective or enhanced potency.

146. The method of claim 143, wherein concurrent administration of (i) and (ii) provides for reduction of resistance in acne-associated bacteria.

147. The method of claim 143, wherein concurrent administration of (i) and (ii) provides for reversal of resistance in acne-associated bacteria.

148. The method of claim 112, wherein the composition is for repeated administration.

149. A method of cosmetic treatment, which method comprises the step of administering to a subject XMP.629 or a physiologically acceptable salt or derivative thereof.

150. The method of claim 149 wherein the administration is effective for cosmetically treating skin.

151. The method of claim 150 wherein the administration is effective for cosmetically improving the clarity of skin.

152. The method of claim 150 wherein the administration is effective for decreasing redness of skin.

153. The method of claim 112, wherein the composition is for repeated administration.

154. A method for reducing or reversing resistance or development of resistance of an acne-associated bacterium to at least one anti-acne agent, comprising administering to a subject with acne a composition comprising a therapeutically effective amount of XMP.629 or a pharmaceutically acceptable salt or derivative thereof, wherein the anti-acne agent is not XMP.629 or a physiologically or pharmaceutically acceptable salt or derivative thereof.

155. A method for reducing or reversing resistance or development of resistance of an acne-associated bacterium to at least one anti-acne agent, comprising
(a) selecting a subject with acne; and
(b) administering to the subject a composition comprising a therapeutically effective amount of XMP.629 or a pharmaceutically acceptable salt or derivative thereof, wherein the anti-acne agent is not XMP.629 or a physiologically or pharmaceutically acceptable salt or derivative thereof.

156. A composition comprising XMP.629 or a pharmaceutically acceptable salt or derivative thereof and further comprising one or more of the following: poloxamer surfactant; ethylenediaminetetraacetic acid (EDTA); benzalkonium chloride (BAK); propylene glycol; and/or hydroxyethylcellulose.

157. The composition of claim 156, wherein the EDTA is in the form of a salt.

158. The composition of claim 157, wherein the EDTA is EDTA disodium, dihydrate.

159. The composition of claim 158, wherein the EDTA disodium, dihydrate is present in the composition at an amount of about 0.15 percent (weight to weight).

160. The composition of claim 156, wherein the propylene glycol is present in the composition at an amount of up to about 20 percent (weight to weight).

161. The composition of claim 156, wherein the XMP.629 or pharmaceutically acceptable salt or derivative thereof is present in the composition at an amount ranging from about 0.01 to about 0.10 percent (weight to weight).

162. The composition of claim 156, wherein the poloxamer surfactant is present in the composition at an amount of about 0.20 percent (weight to weight).

163. The composition of claim 148, wherein the poloxamer surfactant is poloxamer 333.

164. The composition of claim 156, wherein the composition comprises hydroxyethylcellulose.

165. The composition of claim 164, wherein the hydroxyethylcellulose is present in the composition at an amount ranging from about 1.25 to about 1.50 percent (weight to weight).

166. The composition of claim 156, wherein the pharmaceutically acceptable salt is acetate.

167. The composition of claim 156, wherein the composition is in an oral, parental, or topical formulation.

168. The composition of claim 167, wherein the composition is in a topical formulation.

169. The composition of claim 168, wherein the topical formulation is a cream, gel, lotion, or solution.

170. The composition of claim 168, wherein the topical formulation is a cream.

171. The composition of claim 168, wherein the topical formulation is a gel.

172. The composition of claim 168, wherein the topical formulation is a lotion.

173. The composition of claim 168, wherein the topical formulation is a solution.

174. The composition of claim 168, wherein the topical formulation is presented in an impregnated dressing.

175. The composition of claim 168, wherein the topical formulation is presented in a patch.

176. The composition of claim 168, wherein the topical formulation is presented in a gel stick.

177. The composition of claim 168, wherein the topical formulation is presented in a spray.

178. The composition of claim 168, wherein the topical formulation is presented in an aerosol.

179. The composition of claim 168, wherein the topical formulation is presented in a wipe.

180. The composition of claim 168, wherein the topical formulation is presented in a swab.

181. The composition of claim 156, wherein the XMP.629 or pharmaceutically acceptable salt or derivative thereof is present in the composition at an amount ranging ranges from about 0.005% to about 0.5% (weight to volume).

182. The composition of claim 181, wherein the XMP.629 or pharmaceutically acceptable salt or derivative thereof is present in the composition at an amount ranging from about 0.01% to about 0.2% (weight to volume).

183. The composition of claim 182, wherein the XMP.629 or pharmaceutically acceptable salt or derivative thereof is present in the composition at an amount ranging from about 0.01% to about 0.1% (weight to volume).

184. The composition of claim 183, wherein the XMP.629 or pharmaceutically acceptable salt or derivative thereof is present in the composition at an amount ranging from about 0.05% to about 0.1% (weight to volume).

185. The composition of claim 181, wherein the XMP.629 or pharmaceutically acceptable salt or derivative thereof is present in the composition at an amount of about 0.01% (weight to volume).

186. The composition of claim 181, wherein the XMP.629 or pharmaceutically acceptable salt or derivative thereof is present in the composition at an amount of about 0.05% (weight to volume).

187. The composition of claim 181, wherein the XMP.629 or pharmaceutically acceptable salt or derivative thereof is present in the composition at an amount of about 0.1% (weight to volume).

188. The composition of claim 156, further comprising at least one anti-acne agent, wherein the anti-acne agent is not XMP.629 or a pharmaceutically acceptable salt or derivative thereof.

189. The composition of claim 188, wherein the anti-acne agent is in a topical formulation.

190. The composition of claim 189, wherein the topical formulation is a cream, gel, lotion, or solution.

191. The composition of claim 189, wherein the topical formulation is a cream.

192. The composition of claim 189, wherein the topical formulation is a gel.

193. The composition of claim 189, wherein the topical formulation is a lotion.

194. The composition of claim 189, wherein the topical formulation is a solution.

195. The composition of claim 188, wherein the anti-acne agent is a prescription based or over-the-counter agent.

196. The composition of claim 195, wherein the prescription based anti-acne agent is selected from one of the following: benzoyl peroxide, retinoids, retinoid derivatives, antimicrobial agents, or combinations thereof.

197. The method of claim 196, wherein the benzoyl peroxide is administered in an amount ranging from about 2.5% to about 10%.

198. The composition of claim 196, wherein the retinoid is tretinoin.

199. The method of claim 198, wherein the tretinoin is administered in an amount ranging from about 0.01% to about 0.025%.

200. The composition of claim 196, wherein the retinoid derivative is acetylenic retinoid or naphtholic acid retinoid.

201. The composition of claim 196, wherein the antimicrobial agent is azelaic acid.

202. The composition of claim 196, wherein the antibiotic is clindamycin, tetracycline, doxycycline, or erythromycin.

203. The composition of claim 196, wherein the antibiotic combination is benzoyl peroxide with clindamycin, tetracycline, doxycycline, or erythromycin.

204. The method of claim 194, wherein the benzoyl peroxide is administered in an amount ranging from about 2.5% to about 10%.

205. The composition of claim 180, wherein the topical formulation is presented in an impregnated dressing.

206. The composition of claim 180, wherein the topical formulation is presented in a patch.

207. The composition of claim 180, wherein the topical formulation is presented in a gel stick.

208. The composition of claim 180, wherein the topical formulation is presented in a spray.

209. The composition of claim 180, wherein the topical formulation is presented in an aerosol.

210. The composition of claim 180, wherein the topical formulation is presented in a wipe.

211. The composition of claim 180, wherein the topical formulation is presented in a swab.

212. A cosmetic composition according to claim 156.

213. A pharmaceutical composition according to claim 156.

214. A cream comprising the composition of claim 156.

215. A gel comprising the composition of claim 156.

216. A lotion comprising the composition of claim 156.

217. A solution comprising the composition of claim 156.

218. A patch comprising the composition of claim 156.

219. An impregnated dressing comprising the composition of claim 156.

220. A gel stick comprising the composition of claim 156.

221. A spray comprising the composition of claim 156.

222. An aerosol comprising the composition of claim 156.

223. A swab comprising the composition of claim 156.

224. A wipe comprising the composition of claim 156.

225. A composition comprising XMP.629 or a physiologically or pharmaceutically acceptable salt or derivative thereof and further comprising at least one of a chelating agent, tonicity agent, gelling agent, buffer, surfactant, or preservative.

226. A kit comprising (i) XMP.629 or a physiologically or pharmaceutically acceptable salt or derivative thereof and (ii) at least one anti-acne agent, wherein the anti-acne agent is not XMP.629 or a physiologically or pharmaceutically acceptable salt or derivative thereof, for sequential or simultaneous administration to a subject in a method of ameliorating or treating acne.

227. An article containing XMP.629 or a physiologically or pharmaceutically acceptable salt or derivative thereof.

228. The articles of claim 227, further comprising at least one anti-acne agent, wherein the anti-acne agent is not XMP.629 or a physiologically or pharmaceutically salt or derivative thereof, for sequential or simultaneous administration to a subject in a method of ameliorating or treating acne.


SEQUENCE LISTING (25 sequences)

SEQ ID NO: 1 - protein, 9 aa, Homo Sapiens
  MISC_FEATURE: XMP.629
KLFRAQAKA

SEQ ID NO: 2 - protein, 14 aa, Homo Sapiens
  MISC_FEATURE: XMP.624
KLFRAQAKAKGSIK

SEQ ID NO: 3 - protein, 13 aa, Homo Sapiens
  MISC_FEATURE: XMP. 625
KLFRAQAKAKGSI

SEQ ID NO: 4 - protein, 12 aa, Homo Sapiens
  MISC_FEATURE: XMP.626
KLFRAQAKAKGS

SEQ ID NO: 5 - protein, 11 aa, Homo Sapiens
  MISC_FEATURE: XMP.627
KLFRAQAKAKG

SEQ ID NO: 6 - protein, 10 aa, Homo Sapiens
  MISC_FEATURE: XMP.628
KLFRAQAKAK

SEQ ID NO: 7 - protein, 8 aa, Homo Sapiens
  MISC_FEATURE: XMP. 630
KLFRAQAK

SEQ ID NO: 8 - protein, 14 aa, Homo Sapiens
  MISC_FEATURE: XMP.656
KLFRAQAKAKGIKI

SEQ ID NO: 9 - protein, 11 aa, Homo Sapiens
  MISC_FEATURE: XMP.679
KLFKAQAKAKG

SEQ ID NO: 10 - protein, 9 aa, Homo Sapiens
  MISC_FEATURE: XMP.684
AKLFRAQAK

SEQ ID NO: 11 - protein, 8 aa, Homo Sapiens
  MISC_FEATURE: XMP.685
KLFRAQAK

SEQ ID NO: 12 - protein, 8 aa, Homo Sapiens
  MISC_FEATURE: XMP.725
KLFRAQAK

SEQ ID NO: 13 - protein, 11 aa, Homo Sapiens
  MISC_FEATURE: XMP.728
KLFKAQAKAKG

SEQ ID NO: 14 - protein, 9 aa, Homo Sapiens
  MISC_FEATURE: XMP.760
KAFRAQAKA

SEQ ID NO: 15 - protein, 11 aa, Homo Sapiens
  MISC_FEATURE: XMP.764
KAFKAQAKAKG

SEQ ID NO: 16 - protein, 9 aa, Homo Sapiens
  MISC_FEATURE: XMP.776
KLFKAQAKA

SEQ ID NO: 17 - protein, 9 aa, Homo Sapiens
  MISC_FEATURE: XMP.778
KXFRAQAKA

SEQ ID NO: 18 - protein, 8 aa, Homo Sapiens
  MISC_FEATURE: XMP.661
KLFRAQAK

SEQ ID NO: 19 - protein, 8 aa, Homo Sapiens
  MISC_FEATURE: XMP.664
KLFRAQAK

SEQ ID NO: 20 - protein, 8 aa, Homo Sapiens
  MISC_FEATURE: XMP.666
KLFRAQAK

SEQ ID NO: 21 - protein, 8 aa, Homo Sapiens
  MISC_FEATURE: XMP.671
KLFRAQAK

SEQ ID NO: 22 - protein, 8 aa, Homo Sapiens
  MISC_FEATURE: XMP.699
KLFRAQAK

SEQ ID NO: 23 - protein, 8 aa, Homo Sapiens
  MISC_FEATURE: XMP.767
KLFKAQAK

SEQ ID NO: 24 - protein, 8 aa, Homo Sapiens
  MISC_FEATURE: XMP.768
KLFRAQAK

SEQ ID NO: 25 - protein, 8 aa, Homo Sapiens
  MISC_FEATURE: XMP.769
KLFKAQAK